(19) United States
(12) Patent Application Publication
(10) Pub. No.: US 2023/0306267 A1
(43) Pub. Date: Sep. 28, 2023

(54) LEARNING REPRESENTATIONS OF EEG SIGNALS WITH SELF-SUPERVISED LEARNING

(71) Applicant: INTERAXON INC., Toronto (CA)

(72) Inventors: Hubert JACOB BANVILLE, Toronto (CA); Christopher Allen AIMONE, Scarborough (CA); Sean Ulrich Niethe WOOD, Toronto (CA)

(21) Appl. No.: 18/018,823

(22) PCT Filed: Jul. 30, 2021
(86) PCT No.: PCT/CA2021/051079

§ 371 Date: Jan. 30, 2023

Related U.S. Application Data
(60) Provisional application No. 63/058,793, filed on Jul. 30, 2020.

(51) Int. Cl. G06N 3/0895; G06N 3/045

(52) U.S. Cl. CPC G06N 3/0895; G06N 3/045; G16H 50/20

(57) ABSTRACT

Self-supervised learning (SSL) is used to leverage structure in unlabeled data, to learn representations of EEG signals. Two tasks based on temporal context prediction as well as contrastive predictive coding are applied to two clinically-relevant problems: EEG-based sleep staging and pathology detection. Experiments are performed on two large public datasets with thousands of recordings and perform baseline comparisons with purely supervised and hand-engineered paradigms.

### CROSS-REFERENCE

This application claims all benefit including priority to U.S. Provisional Patent Application 63/058,793, filed Jul. 30, 2020, and entitled “LEARNING REPRESENTATIONS OF EEG SIGNALS WITH SELF-SUPERVISED LEARNING”; the entire contents of which are hereby incorporated by reference herein.

### FIELD

Embodiments of the present disclosure generally relate to the field of self-supervised machine learning, and more specifically, embodiments relate to devices, systems and methods for improved training of neural networks on partial or non-annotated training data for bio-signal classification.

### INTRODUCTION

Electroencephalography (EEG) and other bio-signal modalities have enabled numerous applications inside and outside of the clinical domain, e.g., studying sleep patterns and their disruption, monitoring seizures and brain-computer interfacing. In the last few years, the availability and portability of these devices have increased dramatically, effectively democratizing their use and unlocking the potential for positive impact on people's lives. For instance, applications such as at-home sleep staging and apnea detection, pathological EEG detection, mental workload monitoring, etc. are now entirely possible.

In all these scenarios, monitoring modalities generate an ever-increasing amount of data which needs to be interpreted. Therefore, predictive models that can classify, detect and ultimately “understand” physiological data are required. Modelling can use supervised approaches, where large datasets of annotated examples are required to train models with high performance.

However, obtaining accurate annotations on physiological data can prove expensive, time consuming or may be impossible. For example, annotating sleep recordings requires trained technicians to go through hours of data visually and label 30-s windows one-by-one. Clinical recordings such as those used to diagnose epilepsy or brain lesions must be reviewed by neurologists, who might not always be available. More broadly, noise in the data and the complexity of brain processes of interest can make it difficult to interpret and annotate EEG signals, which can lead to high inter-rater variability, i.e., label noise. Furthermore, in some cases, knowing exactly what the participants were thinking or doing in cognitive neuroscience experiments can be challenging, making it difficult to obtain accurate labels. In imagery tasks, for instance, the subjects might not be following instructions or the process under study might be difficult to quantify objectively (e.g., meditation, emotions). Therefore, a new paradigm that does not rely primarily on supervised learning is necessary for making use of large unlabeled sets of recordings such as those generated in the scenarios described above. However, traditional unsupervised learning approaches such as clustering and latent factor models do not offer fully satisfying answers as their performance is not as straightforward to quantify and interpret as supervised ones.

### SUMMARY

Supervised learning paradigms are often limited by the amount of labeled data that is available. This may be problematic when working with clinically relevant data, such as electroencephalography (EEG), where labeling can be costly in terms of specialized expertise and human processing time. Consequently, deep learning architectures designed to learn on EEG data have had to take this limitation into account, yielding relatively shallow models and performances that may be similar to those of traditional feature-based approaches. However, in most situations, unlabeled data is available in abundance. By extracting information from this unlabeled data, it might be possible to achieve competitive performance with deep neural network performance with limited access to labels. In this context, one promising technique is self-supervised learning (SSL). In some embodiments of the present disclosure, different SSL approaches are provided to learn representations for EEG signals. Specifically, two tasks are explored, based on temporal context prediction as well as contrastive predictive coding on two clinically relevant problems: EEG-based sleep staging and pathology detection. Experiment results were obtained with two large public datasets encompassing thousands of recordings. Baseline comparisons are also conducted with purely supervised and hand-engineered approaches. Finally, it is illustrated how the embedding learned with each method reveals interesting latent structures in the data, such as age effects. Studies described herein demonstrate the benefit of SSL approaches on EEG data. Experiment results herein suggest that SSL may pave the way to a wider use of deep learning models on EEG data. The experiments are non-limiting examples to illustrative aspects of embodiments described herein.

In accordance with an aspect, there is provided a system for training a neural network to classify bio-signal data by updating trainable parameters of the neural network. The system has a memory and a training computing apparatus. The memory is configured to store training bio-signal data from one or more subjects. The training bio-signal data includes labeled training bio-signal data and unlabeled training bio-signal data. The training computing apparatus is configured to receive the training bio-signal data from memory, define one or more sets of time windows within the training bio-signal data, each set including a first anchor window and a sampled window, for at least one set of the one or more sets, determine a determined set representation based in part on the relative position of the first anchor window and the sampled window, extract a feature representation of the first anchor window and a feature representation of the sampled window using an embedder neural network, aggregate the feature representations using a contrastive module, and predict a predicted set representation using the aggregated feature representations, update trainable parameters of the embedder neural network to minimize a difference between the determined set representation of the at least one set and the predicted set representation of the at least one set, and label the unlabeled training bio-signal data using a classifier, the labeled training bio-signal data, and the embedder neural network. The set representation denotes likely label correspondence between the first anchor window and the sampled window.

Some embodiments further include a bio-signal sensor and a classifying computing apparatus. The bio-signal sensor is configured to receive user bio-signal data from a user. The classifying computing apparatus is configured to receive the embedder neural network from the training computing apparatus, receive the user bio-signal data from the bio-signal sensor, and label the user bio-signal data using the embedder neural network and the classifier.

In some embodiments, the training computing apparatus is further configured to store the training bio-signal data labelled using the classifier in the memory. In some embodiments, the training computing apparatus is further configured to transmit the training bio-signal data labelled using the classifier to another computing apparatus.

In some embodiments, the one or more sets of time windows include one or more pairs of time windows, the at least one set of the one or more sets include at least one pair of the one or more pairs, and the training computing apparatus determines the determined set representation based in part on the relative position of the first anchor window and the sampled window by defining a positive context region and negative context region surrounding the first anchor window, and determining if the sampled window is within the positive context region or negative context region.

In some embodiments, the determined set representation is based in part on the relative position of the first anchor window and the sampled window further includes rejecting the at least one pair if the sampled window is not within the positive context region or the negative context region.

In some embodiments, the one or more sets of time windows include one or more triplets of time windows, each triplet further including a second anchor window, wherein the second anchor window is within a positive context region surrounding the first anchor window, the at least one set of the one or more sets includes at least one triplet of the one or more triplets, the training computing apparatus determines the determined set representation based in part on the relative position of the first anchor window and the sampled window by determining a temporal order of the first anchor window, the sampled window, and the second anchor window, and the extract the feature representation of the first anchor window and a feature representation of the sampled window using an embedder neural network further includes extracting a feature representation of the second anchor window.

In some embodiments, define the one or more triplets of time windows within the training bio-signal data further includes for one triplet of the one or more triplets, a mirror of the one triplet is included in the one or more triplets.

In some embodiments, the first anchor window includes a series of consecutive anchor windows, the sampled window includes a series of consecutive sampled windows, wherein the series of consecutive sampled windows is adjacent to the series of consecutive anchor windows, the set further includes a set of negative sample windows, and the training computing apparatus determines the determined set representation based in part on the relative position of the first anchor window and the sampled window by determining that a given sampled window is in the series of sampled windows. In such embodiments, the extract the feature representation of the first anchor window and a feature representation of the sampled window using an embedder neural network includes extracting a feature representation of each anchor window of the series of consecutive anchor windows, extracting a feature representation of each sampled window of the series of consecutive sampled windows, and extracting a feature representation of each negative sample window of the set of negative sample windows. In such embodiments, the aggregate the feature representations includes embedding the feature representation of each anchor window of the series of anchor windows using an autoregressive embedder, and aggregating the embedded anchor series, a given feature representation of a given sampled window of the series of sampled windows, and one or more given feature representations of one or more given negative sample windows of the set of negative sample windows. In such embodiments, the predict the predicted set representation includes predicting which of the given feature representations corresponds to the given feature representation of the sampled window of the series of sampled windows.

In some embodiments, the embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the label the user bio-signal data includes determining a brain state of the user. In some embodiments, the label the user bio-signal data includes determining a sleep state of the user. In some embodiments, the label the user bio-signal data includes detecting a pathology of the user.

In some embodiments, the training computing apparatus includes a server having at least one hardware processor. In some embodiments, the training computing apparatus includes a server configured to upload the embedder neural network and the classifier to the classifier computing apparatus. In some embodiments, the training computing apparatus includes the classifying computing apparatus.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network.

In some embodiments, the classifier labels unlabeled bio-signal data based in part on a personal characteristic of the subject corresponding to the training bio-signal data, and the classifying the user bio-signal data is based in part on a personal characteristic of the user.

In accordance with another aspect, there is provided a method for training a neural network to classify bio-signal data by updating trainable parameters of the neural network. The method includes receiving training bio-signal data from one or more subjects including labeled training bio-signal data and unlabeled training bio-signal data, defining one or more sets of time windows within the training bio-signal data, each set including a first anchor window and a sampled window, for at least one set of the one or more sets, determining a determined set representation based in part on the relative position of the first anchor window and the sampled window, extracting a feature representation of the first anchor window and a feature representation of the sampled window using an embedder neural network, aggregating the feature representations using a contrastive module, predicting a predicted set representation using the aggregated feature representations, updating trainable parameters of the embedder neural network to minimize a difference between the determined set representation of the at least one set and the predicted set representation of the at least one set, and labeling the unlabeled training bio-signal data using a classifier, the labeled training bio-signal data, and the embedder neural network. The set representation denotes likely label correspondence between the first anchor window and the sampled window.

Some embodiments further include receiving user bio-signal data from a user using a bio-signal sensor, and labeling the user bio-signal data using the embedder neural network and the classifier.

Some embodiments further comprise storing the training bio-signal data labelled using the classifier in the memory. Some embodiments further comprise transmitting the training bio-signal data labelled using the classifier to another computing apparatus.

In some embodiments, the one or more sets of time windows are one or more pairs of time windows, the at least one set of the one or more sets are at least one pair of the one or more pairs, the determining a determined set representation based in part on the relative position of the first anchor window and the sampled window includes defining a positive context region and negative context region surrounding the first anchor window, and determining if the sampled window is within the positive context region or negative context region.

In some embodiments, the determining a determined set representation based in part on the relative position of the first anchor window and the sampled window further includes rejecting the at least one pair if the sampled window is not within the positive context region or the negative context region.

In some embodiments, the one or more sets of time windows are one or more triplets of time windows, each triplet further includes a second anchor window, wherein the second anchor window is within a positive context region surrounding the first anchor window, the at least one set of the one or more sets is at least one triplet of the one or more triplets, the determining a determined set representation based in part on the relative position of the first anchor window and the sampled window includes determining a temporal order of the first anchor window, the sampled window, and the second anchored window, and the extract a feature representation of the first anchor window and a feature representation of the sampled window using an embedder neural network further includes extracting a feature representation of the second anchor window.

In some embodiments, defining one or more triplets of time windows within the training bio-signal data further includes for one triplet of the one or more triplets, a mirror of the one triplet is included in the one or more triplets.

In some embodiments, the first anchor window includes a series of consecutive anchor windows, the sampled window includes a series of consecutive sampled windows, wherein the series of consecutive sampled windows is adjacent to the series of consecutive anchor windows, the set further includes a set of negative sample windows, the determining a determined set representation based in part on the relative position of the first anchor window and the sampled window includes determining that a given sampled window is in the series of sampled windows. In such embodiments, the extracting a feature representation of the first anchor window and a feature representation of the sampled window using an embedder neural network further includes extracting a feature representation of each anchor window of the series of consecutive anchor windows, extracting a feature representation of each sampled window of the series of consecutive sampled windows, and extracting a feature representation of each negative sample window of the set of negative sample windows. In such embodiments, the aggregating the feature representations includes embedding the feature representation of each anchor window of the series of anchor windows using an autoregressive embedder, and aggregating the embedded anchor series, a given feature representation of a given sampled window of the series of sampled windows, and one or more given feature representations of one or more given negative sample windows of the set of negative sample windows. In such embodiments, the predicting a predicted set representation includes predicting which of the given feature representations corresponds to the given feature representation of the sampled window of the series of sampled windows.

In some embodiments, the embedder neural network includes one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the labeling the user bio-signal data includes determining a brain state of the user. In some embodiments, the labeling the user bio-signal data includes determining a sleep state of the user. In some embodiments, the labeling the user bio-signal data includes detecting a pathology of the user.

Some embodiments further including uploading the embedder neural network to a server.

In some embodiments, the contrastive module includes a contrastive neural network, and the updating trainable parameters further includes updating trainable parameters of the contrastive neural network.

In some embodiments, the classifier includes a classifier neural network, and the update trainable parameters includes updating trainable parameters of the classifier neural network.

In some embodiments, the classifier labels unlabeled bio-signal data based in part on a personal characteristic of the subject corresponding to the training bio-signal data, and the classifying the user bio-signal data is based in part on a personal characteristic of the user.

In accordance with another aspect, there is provided a system for training a neural network to classify bio-signal data by updating trainable parameters of the neural network. The system has a memory and a training computing apparatus. The memory is configured to store training bio-signal data from one or more subjects. The training bio-signal data includes labeled training bio-signal data and unlabeled training bio-signal data. The training computing apparatus is configured to receive the training bio-signal data from the memory, define one or more pairs of time windows within the training bio-signal data, each pair including an anchor window and a sampled window, for at least one pair of the one or more pairs, define a positive context region and negative context region surrounding the anchor window, determine a determined pair representation based on whether the sampled window is within the positive context region or negative context region, extract a feature representation of the anchor window and a feature representation of the sampled window using an embedder neural network, aggregate the feature representations using a contrastive module, and predict a predicted pair representation using the aggregated feature representations, update trainable parameters of the embedder neural network to minimize a difference between the determined pair representation of the at least one pair and the predicted pair representation of the at least one pair, and label the unlabeled training bio-signal data using a classifier, the labeled training bio-signal data, and the embedder neural network. The pair representation denotes likely label correspondence between the anchor window and the sampled window.

Some embodiments further include a bio-signal sensor and a classifying computing apparatus. The bio-signal sensor is configured to receive user bio-signal data from a user. The classifying computing apparatus is configured to receive the embedder neural network from the training computing apparatus, receive the user bio-signal data from the bio-signal sensor, and label the user bio-signal data using the embedder neural network and the classifier.

In some embodiments, the training computing apparatus is further configured to store the training bio-signal data labelled using the classifier in the memory. In some embodiments, the training computing apparatus is further configured to transmit the training bio-signal data labelled using the classifier to another computing apparatus.

In some embodiments, the determined set representation is based in part on the relative position of the anchor window and the sampled window further includes rejecting the at least one pair if the sampled window is not within the positive context region or the negative context region.

In some embodiments, the embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the label the user bio-signal data includes determining a brain state of the user. In some embodiments, the label the user bio-signal data includes determining a sleep state of the user. In some embodiments, the label the user bio-signal data includes detecting a pathology of the user.

In some embodiments, the training computing apparatus includes a server having at least one hardware processor. In some embodiments, training computing apparatus includes a server configured to upload the embedder neural network and the classifier to the classifier computing apparatus. In some embodiments, the training computing apparatus includes classifying computing apparatus.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network.

In some embodiments, the classifier labels unlabeled bio-signal data based in part on a personal characteristic of the subject corresponding to the training bio-signal data, and the classifying the user bio-signal data is based in part on a personal characteristic of the user.

In accordance with another aspect, there is provided a system for training a neural network to classify bio-signal data by updating trainable parameters of the neural network. The system includes a memory and a training computing apparatus. The memory is configured to store training bio-signal data from one or more subjects. The training bio-signal data includes labeled training bio-signal data and unlabeled training bio-signal data. The training computing apparatus is configured to receive the training bio-signal data from the memory, define one or more triplets of time windows within the training bio-signal data, each triplet including a first anchor window, a second anchor window, and a sampled window, for at least one triplet of the one or more triplets, determine a determined triplet representation based in part on the temporal order of the first anchor window, the second anchor window, and the sampled window, extract a feature representation of the first anchor window, a feature representation of the second anchor window, and a feature representation of the sampled window using an embedder neural network, aggregate the feature representations using a contrastive module, and predict a predicted triplet representation using the aggregated feature representations, update trainable parameters of the embedder neural network to minimize a difference between the determined triplet representation of the at least one triplet and the predicted triplet representation of the at least one triplet, and label the unlabeled training bio-signal data using classifier, the labeled training bio-signal data, and the embedder neural network. The second anchor window is within a positive context region surrounding the first anchor window. The triplet representation denotes likely label correspondence between the first anchor window and the sampled window.

Some embodiments further include a bio-signal sensor and a classifying computing apparatus. The bio-signal sensor is configured to receive user bio-signal data from a user. The classifying computing apparatus is configured to receive the embedder neural network from the training computing apparatus, receive the user bio-signal data from the bio-signal sensor, and label the user bio-signal data using the embedder neural network and the classifier.

In some embodiments, the training computing apparatus is further configured to store the training bio-signal data labelled using the classifier in the memory. In some embodiments, the training computing apparatus is further configured to transmit the training bio-signal data labelled using the classifier to another computing apparatus.

In some embodiments, the define the one or more triplets of time windows within the training bio-signal data further includes for one triplet of the one or more triplets, a mirror of the one triplet is included in the one or more triplets.

In some embodiments, embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the label the user bio-signal data includes determining a brain state of the user. In some embodiments, the label the user bio-signal data includes determining a sleep state of the user. In some embodiments, the label the user bio-signal data includes detecting a pathology of the user.

In some embodiments, the training computing apparatus includes a server having at least one hardware processor. In some embodiments, the training computing apparatus includes a server configured to upload the embedder neural network and the classifier to the classifier computing apparatus. In some embodiments, the training computing apparatus includes the classifying computing apparatus.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network.

In some embodiments, the classifier labels unlabeled bio-signal data based in part on a personal characteristic of the subject corresponding to the training bio-signal data, and the classifying the user bio-signal data is based in part on a personal characteristic of the user.

In accordance with another aspect, there is provided a system for training a neural network to classify bio-signal data by updating trainable parameters of the neural network. The system has a memory and a training computing apparatus. The memory is configured to store training bio-signal data from one or more subjects. The training bio-signal data includes labeled training bio-signal data and unlabeled training bio-signal data. The training computing apparatus is configured to receive the training bio-signal data from the memory, define one or more sets of time windows within the training bio-signal data, each set including a series of consecutive anchor windows, a series of consecutive sampled windows, and a set of negative sample windows, for at least one set of the one or more sets, extract a feature representation of each anchor window of the series of consecutive anchor windows, a feature representation of each sampled window of the series of consecutive sampled windows, and a feature representation of each negative sample window of the set of negative sample windows using an embedder neural network, embed the feature representation of each anchor window of the series of anchor windows using an autoregressive embedder, aggregate the embedded feature representation of each anchor window, a given feature representation of a given sampled window, and one or more given feature representations of one or more given negative sample windows using a contrastive module, and predict which of the given sampled window and the one or more given negative sample windows is the given sampled window based on the aggregated feature representations, update trainable parameters of the embedder neural network to minimize predictions that predict the one or more given negative sample windows is the given sampled window, and label the unlabeled training bio-signal data using a classifier, the labeled training bio-signal data, and the embedder neural network. The series of consecutive sampled windows is adjacent to the series of consecutive anchor windows.

Some embodiments further include a bio-signal sensor and a classifying computing apparatus. The bio-signal sensor is configured to receive user bio-signal data from a user. The classifying computing apparatus is configured to receive the embedder neural network from the training computing apparatus, receive the user bio-signal data from the bio-signal sensor, and label the user bio-signal data using the embedder neural network and the classifier.

In some embodiments, the training computing apparatus is further configured to store the training bio-signal data labelled using the classifier in the memory. In some embodiments, the training computing apparatus is further configured to transmit the training bio-signal data labelled using the classifier to another computing apparatus.

In some embodiments, the embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the label the user bio-signal data includes determining a brain state of the user. In some embodiments, the label the user bio-signal data includes determining a sleep state of the user. In some embodiments, the label the user bio-signal data includes detecting a pathology of the user.

In some embodiments, the training computing apparatus includes a server having at least one hardware processor. In some embodiments, the training computing apparatus includes a server configured to upload the embedder neural network and the classifier to the classifier computing apparatus. In some embodiments, the training computing apparatus includes the classifying computing apparatus.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network.

In some embodiments, the classifier labels unlabeled bio-signal data based in part on a personal characteristic of the subject corresponding to the training bio-signal data, and the classifying the user bio-signal data is based in part on a personal characteristic of the user.

In accordance with another aspect, there is provided a system for classifying bio-signal data by updating trainable parameters of a neural network. The system has a memory and a computing apparatus. The memory is configured to store bio-signal data from one or more subjects. The computing apparatus is configured to receive the bio-signal data from the memory, define one or more sets of time windows within the bio-signal data, each set including a first anchor window and a sampled window, for at least one set of the one or more sets, determine a determined set representation based in part on the relative position of the first anchor window and the sampled window, extract a feature representation of the first anchor window and a feature representation of the sampled window using an embedder neural network, aggregate the feature representations using a contrastive module, and predict a predicted set representation using the aggregated feature representations, update trainable parameters of the embedder neural network to minimize a difference between the determined set representation of the at least one set and the predicted set representation of the at least one set, correspond at least one time window within the bio-signal data with at least one other time window within the bio-signal data based on the feature representation of the at least one time window and the feature representation of the at least one other time window using the trained embedder neural network, and present corresponded time windows.

In some embodiments, the one or more sets of time windows include one or more pairs of time windows, the at least one set of the one or more sets include at least one pair of the one or more pairs, and the computing apparatus determines the determined set representation based in part on the relative position of the first anchor window and the sampled window includes defining a positive context region and negative context region surrounding the first anchor window, and determining if the sampled window is within the positive context region or negative context region.

In some embodiments, the computing apparatus is further configured to store the corresponded windows in a memory. In some embodiments, the computing apparatus is further configured to transmit corresponded windows to another computing apparatus.

In some embodiments, the determined set representation is based in part on the relative position of the first anchor window and the sampled window further includes rejecting the at least one pair if the sampled window is not within the positive context region or the negative context region.

In some embodiments, the one or more sets of time windows include one or more triplets of time windows, each triplet further including a second anchor window, wherein the second anchor window is within a positive context region surrounding the first anchor window, the at least one set of the one or more sets includes at least one triplet of the one or more triplets, the computing apparatus determines the determined set representation based in part on the relative position of the first anchor window and the sampled window by determining a temporal order of the first anchor window, the sampled window, and the second anchor window, and the extract the feature representation of the first anchor window and a feature representation of the sampled window using an embedder neural network further includes extracting a feature representation of the second anchor window.

In some embodiments, the define the one or more triplets of time windows within the training bio-signal data further includes for one triplet of the one or more triplets, a mirror of the one triplet is included in the one or more triplets.

In some embodiments, the first anchor window includes a series of consecutive anchor windows, the sampled window includes a series of consecutive sampled windows, wherein the series of consecutive sampled windows is adjacent to the series of consecutive anchor windows, the set further includes a set of negative sample windows, and the computing apparatus determines the determined set representation based in part on the relative position of the first anchor window and the sampled window by determining that a given sampled window is in the series of sampled windows. In such embodiments, the extract a feature representation of the first anchor window and the feature representation of the sampled window using an embedder neural network includes extracting a feature representation of each anchor window of the series of consecutive anchor windows, extracting a feature representation of each sampled window of the series of consecutive sampled windows, and extracting a feature representation of each negative sample window of the set of negative sample windows. In such embodiments, the aggregate the feature representations includes embedding the feature representation of each anchor window of the series of anchor windows using an autoregressive embedder, and aggregating the embedded anchor series, a given feature representation of a given sampled window of the series of sampled windows, and one or more given feature representations of one or more given negative sample windows of the set of negative sample windows. In such embodiments, the predict the predicted set representation includes predicting which of the given feature representations corresponds to the given feature representation of the sampled window of the series of sampled windows.

In some embodiments, the embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the computing apparatus includes a server having at least one hardware processor.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network.

Some embodiments further include a display. In such embodiments the present the corresponded time windows includes presenting the corresponded time windows using the display.

In some embodiments, the bio-signal data from one or more subjects further includes associations between personal characteristics of the one or more subjects and the bio-signal data, and the present corresponded time windows includes presenting the personal characteristics associated with the bio-signal data.

In accordance with another aspect, there is provided an apparatus for classifying bio-signal data by updating trainable parameters of the neural network. The apparatus includes a bio-signal sensor and a computing apparatus. Bio-signal sensor is configured to receive bio-signal data from a subject. The bio-signal data includes unlabeled bio-signal data. The computing apparatus is configured to receive the bio-signal data from the subject, define one or more sets of time windows within the bio-signal data, each set including a first anchor window and a sampled window, for at least one set of the one or more sets, determine a determined set representation based in part on the relative position of the first anchor window and the sampled window, extract a feature representation of the first anchor window and a feature representation of the sampled window using an embedder neural network, aggregate the feature representations using a contrastive module, and predict a predicted set representation using the aggregated feature representations, update trainable parameters of the embedder neural network to minimize a difference between the determined set representation of the at least one set and the predicted set representation of the at least one set, present the bio-signal data to a user, receive at least one label from the user via an input to generate one or more labeled windows within the bio-signal data, and label the unlabeled bio-signal data using a classifier, the one or more labeled windows, and the embedder neural network. The set representation denotes likely label correspondence between the first anchor window and the sampled window.

In some embodiments, the computing apparatus is further configured to store the bio-signal data labelled using the classifier in a memory. In some embodiments, the computing apparatus is further configured to transmit the bio-signal data labelled using the classifier to another computing apparatus.

In some embodiments, the one or more sets of time windows include one or more pairs of time windows, the at least one set of the one or more sets include at least one pair of the one or more pairs, and the computing apparatus determines the determined set representation based in part on the relative position of the first anchor window and the sampled window by defining a positive context region and negative context region surrounding the first anchor window, and determining if the sampled window is within the positive context region or negative context region.

In some embodiments, the determined set representation is based in part on the relative position of the first anchor window and the sampled window further includes rejecting the at least one pair if the sampled window is not within the positive context region or the negative context region.

In some embodiments, the one or more sets of time windows include one or more triplets of time windows, each triplet further including a second anchor window, wherein the second anchor window is within a positive context region surrounding the first anchor window, the at least one set of the one or more sets includes at least one triplet of the one or more triplets, the computing apparatus determines the determined set representation based in part on the relative position of the first anchor window and the sampled window by determining a temporal order of the first anchor window, the sampled window, and the second anchor window, and the extract a feature representation of the first anchor window and a feature representation of the sampled window using an embedder neural network further includes extracting a feature representation of the second anchor window.

In some embodiments, define the one or more triplets of time windows within the training bio-signal data further includes for one triplet of the one or more triplets, a mirror of the one triplet is included in the one or more triplets.

In some embodiments, the first anchor window includes a series of consecutive anchor windows, the sampled window includes a series of consecutive sampled windows, wherein the series of consecutive sampled windows is adjacent to the series of consecutive anchor windows, the set further includes a set of negative sample windows, and the computing apparatus determines the determined set representation based in part on the relative position of the first anchor window and the sampled window by determining that a given sampled window is in the series of sampled windows. In such embodiments, the extract the feature representation of the first anchor window and a feature representation of the sampled window using an embedder neural network includes extracting a feature representation of each anchor window of the series of consecutive anchor windows, extracting a feature representation of each sampled window of the series of consecutive sampled windows, and extracting a feature representation of each negative sample window of the set of negative sample windows. In such embodiments, the aggregate the feature representations includes embedding the feature representation of each anchor window of the series of anchor windows using an autoregressive embedder, and aggregating the embedded anchor series, a given feature representation of a given sampled window of the series of sampled windows, and one or more given feature representations of one or more given negative sample windows of the set of negative sample windows. In such embodiments, the predict the predicted set representation includes predicting which of the given feature representations corresponds to the given feature representation of the sampled window of the series of sampled windows.

In some embodiments, the embedder neural network can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data.

In some embodiments, the label the user bio-signal data includes determining a brain state of the user. In some embodiments, the label the user bio-signal data includes determining a sleep state of the user. In some embodiments, the label the user bio-signal data includes detecting a pathology of the user.

In some embodiments, the training computing apparatus includes a server having at least one hardware processor. In some embodiments, the training computing apparatus includes a server configured to upload the embedder neural network and the classifier to the classifier computing apparatus. In some embodiments, the training computing apparatus includes the classifying computing apparatus.

In some embodiments, the classifier includes a classifier neural network, and the update trainable parameters includes updating trainable parameters of the classifier neural network.

In some embodiments, the contrastive module includes a contrastive neural network, and the update trainable parameters further includes updating trainable parameters of the contrastive neural network

In some embodiments, the classifier labels unlabeled bio-signal data based in part on a personal characteristic of the subject corresponding to the training bio-signal data, and the classifying the user bio-signal data is based in part on a personal characteristic of the user.

In some embodiments, the classifier includes a classifying neural network, and the computing apparatus is further configured to update trainable parameters of the classifying neural network to minimize a difference between labels of the one or more labeled windows in the bio-signal data and predicted labels of the one or more labeled windows in the bio-signal data.

Other features will become apparent from the drawings in conjunction with the following description.

### BRIEF DESCRIPTION OF DRAWINGS

In the figures which illustrate example embodiments:

FIG. 1 illustrates a schematic diagram of a system capable of implementing SSL to train a neural network to label bio-signal data using labeled and unlabeled data, according to some embodiments.

FIG. 2 illustrates a schematic diagram of a system capable of using a neural network to label bio-signal data trained with SSL using labeled and unlabeled data, according to some embodiments.

FIG. 3 illustrates a flowchart showing the example operation of the SSL training operation implemented in FIG. 1, according to some embodiments.

FIG. 4 illustrates a schematic diagram of a system capable of implementing SSL to train a neural network to group bio-signal data using unlabeled data, according to some embodiments.

FIG. 5 illustrates a schematic diagram of a system capable of implementing SSL to train a neural network to label bio-signal data using unlabeled data and bio-signal data labeled by a user during operation, according to some embodiments.

FIG. 6 is a visual explanation of three SSL pretext tasks used herein with FIG. 6A showing Relative Positioning, FIG. 6B showing Temporal Shuffling, and FIG. 6C showing Contrastive Predictive Coding (CPC). The first column of each figure illustrates the sampling process by which examples are obtained in each pretext task. The second column describes the training process, where sampled examples are used to train a feature extractor h_{Θ} end-to-end.

FIG. 7 illustrates neural network architectures used as embedder h_{Θ} for (1) sleep EEG and (2) pathology detection experiments.

FIG. 8A is a table describing the Physionet Challenge 2018 (PC18) dataset used in this study for sleep staging experiments.

FIG. 8B is a table describing the TUH Abnormal (TUHab) dataset used in EEG pathology detection experiments.

FIG. 9 is a table of a number of recordings used in the training, validation and testing sets with PC18 and TUHab, as well as the number of examples for each pretext task.

FIG. 10 is a table of SSL pretext task hyperparameter values considered in training. Bold face indicates values that led to the highest downstream task performance.

FIG. 11 illustrates impact of number of labeled examples per class on downstream performance. Feature extractors were trained with an autoencoder (AE), the relative positioning (RP), temporal shuffling (TS) and contrastive predictive coding (CPC) tasks, or left untrained (‘random weights’), and then used to extract features on PC18 and TUHab. Following a hyperparameter search, the same-recording negative sampling on PC18 and across-recording negative sampling on TUHab was used. Downstream task performance was evaluated by training linear logistic regression models on the extracted features for the labeled examples, with at least one and up to all existing labeled examples in the training set (‘All’). Additionally, fully supervised models were trained directly on labeled data and random forests were trained on handcrafted features. Results are the average of five runs with same initialization but different randomly selected examples. While more labeled examples led to better performance, SSL models achieved much higher performance than a fully supervised model when only few were available.

FIG. 12 illustrates the impact of number of labeled examples per class on downstream performance for a self-training semi-supervised baseline, as compared to the handcrafted features approach. Self-training experiments were conducted with a random forest (RF) and logistic regression (LR) using the same hyperparameters as described in the section titled “Baselines” and a probability threshold of 0.7 or 0.4 and maximum number of iterations of 5. Self-training overall harmed downstream performance for both datasets.

FIG. 13 illustrates UMAP visualization of SSL features on the PC18 dataset. The subplots show the distribution of the 5 sleep stages as scatterplots for TS (first row) and CPC (second row) features. Contour lines correspond to the density levels of the distribution across all stages and are used as visual reference. Finally, each point corresponds to the features extracted from a 30-s window of EEG by the TS and CPC embedders with the highest downstream performance. All available windows from the train, validation and test sets of PC18 were used. In both cases, there is clear structure related to sleep stages although no labels were available during training.

FIG. 14 illustrates structure learned by the embedders trained on the TS task. The models with the highest downstream performance were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with SSL capture physiologically-relevant structure, such as pathology, age, apnea and gender.

FIG. 15 illustrates structure related to the original recording's number of EEG channels and measurement date in TS-learned features on the entire TUHab dataset. The overall different number of EEG channels and measurement date in each cluster shows that the cluster-like structure reflects differences in experimental setups.

FIG. 16 illustrates UMAP visualization of SSL features on the PC18 dataset. The subplots show the distribution of the 5 sleep stages as scatterplots for RP features. Contour lines correspond to the density levels of the distribution across all stages and are used as visual reference. Finally, each point corresponds to the features extracted from a 30-s window of EEG by the RP embedders with the highest downstream performance. All available windows from the train, validation and test sets of PC18 were used.

FIG. 17 illustrates structure learned by the embedders trained on the RP task. The models with the highest downstream performance were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with SSL capture physiologically-relevant structure, such as pathology, age, apnea and gender.

FIG. 18 illustrates structure learned by the embedders trained on the CPC task. The models with the highest downstream performance were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with SSL capture physiologically-relevant structure, such as pathology, age, apnea and gender.

FIG. 19A illustrates the impact of principal hyperparameters on pretext (black, star) and downstream (white, circle) task performance, measured with balanced accuracy on the validation set on PC18. Each row corresponds to a different SSL pretext task. For both RP and TS, the hyperparameters that control the length of the positive and negative contexts (τₚₒₛ, τ_{neg}, in seconds) were varied; the exponent “same” or “all” indicates whether negative windows were sampled across the same recording or across all recordings, respectively. For CPC, the number of predicted windows and the type of negative sampling was varied. Finally, the best hyperparameter values in terms of downstream task performance are emphasized using vertical dashed lines.

FIG. 19B the illustrates impact of principal hyperparameters on pretext (black, star) and downstream (white, circle) task performance, measured with balanced accuracy on the validation set on TUHab. Each row corresponds to a different SSL pretext task. For both RP and TS, the hyperparameters that control the length of the positive and negative contexts (τₚₒₛ, τ_{neg}, in seconds) were varied; the exponent “same” or “all” indicates whether negative windows were sampled across the same recording or across all recordings, respectively. For CPC, the number of predicted windows and the type of negative sampling was varied. Finally, the best hyperparameter values in terms of downstream task performance are emphasized using vertical dashed lines.

FIG. 20 is a block diagram of example hardware and software components of a computing device, according to an embodiment.

### DETAILED DESCRIPTION

Electroencephalography (EEG) and other bio-signal modalities have enabled numerous applications inside and outside of the clinical domain, e.g., studying sleep patterns and their disruption, monitoring seizures and brain-computer interfacing. In the last few years, the availability and portability of these devices have increased dramatically, effectively democratizing their use and unlocking the potential for positive impact on people's lives. For instance, applications such as at-home sleep staging and apnea detection, pathological EEG detection, mental workload monitoring, etc. are now entirely possible.

In all these scenarios, monitoring modalities generate an ever-increasing amount of data which needs to be interpreted. Therefore, predictive models that can classify, detect and ultimately “understand” physiological data are required. Traditionally, this type of modelling has mostly relied on supervised approaches, where large datasets of annotated examples are required to train models with high performance.

However, obtaining accurate annotations on physiological data can prove expensive, time consuming or simply impossible. For example, annotating sleep recordings requires trained technicians to go through hours of data visually and label 30-s windows one-by-one. Clinical recordings such as those used to diagnose epilepsy or brain lesions must be reviewed by neurologists, who might not always be available. More broadly, noise in the data and the complexity of brain processes of interest can make it difficult to interpret and annotate EEG signals, which can lead to high inter-rater variability, i.e., label noise. Furthermore, in some cases, knowing exactly what the participants were thinking or doing in cognitive neuroscience experiments can be challenging, making it difficult to obtain accurate labels. In imagery tasks, for instance, the subjects might not be following instructions or the process under study might be difficult to quantify objectively (e.g., meditation, emotions). Therefore, a new paradigm that does not rely primarily on supervised learning is necessary for making use of large unlabeled sets of recordings such as those generated in the scenarios described above. However, traditional unsupervised learning approaches such as clustering and latent factor models do not offer fully satisfying answers as their performance is not as straightforward to quantify and interpret as supervised ones.

Supervised learning paradigms are often limited by the amount of labeled data that is available. This phenomenon is particularly problematic when working with clinically relevant data, such as electroencephalography (EEG), where labeling can be costly in terms of specialized expertise and human processing time. Consequently, deep learning architectures designed to learn on EEG data have had to take this limitation into account, yielding relatively shallow models and performances that are at best similar to those of traditional feature-based approaches. However, in most situations, unlabeled data is available in abundance. By extracting information from this unlabeled data, it might be possible to achieve competitive performance with deep neural network performance with limited access to labels. In this context, one promising technique is self-supervised learning (SSL). In some embodiments of the present disclosure, different SSL approaches are provided to learn representations for EEG signals. Specifically, two tasks are explored, based on temporal context prediction as well as contrastive predictive coding on two clinically relevant problems: EEG-based sleep staging and pathology detection. Results are obtained with two large public datasets encompassing thousands of recordings. Baseline comparisons are also conducted with purely supervised and hand-engineered approaches. Finally, it is illustrated how the embedding learned with each method reveals interesting latent structures in the data, such as age effects. Studies described herein demonstrate the benefit of self-supervised learning approaches on EEG data. Results herein suggest that SSL may pave the way to a wider use of deep learning models on EEG data.

“Self-supervised learning” (SSL) is an unsupervised learning approach that learns representations from unlabeled data, exploiting the structure of the data to provide supervision. By reframing an unsupervised learning problem as a supervised one, SSL allows the use of standard, better understood optimization procedures. SSL comprises a “pretext” and a “downstream” task. The downstream task is the task one is actually interested in but for which there are limited or no annotations. The pretext task, on the other hand, should be sufficiently related to the downstream task such that similar representations should be employed to carry it out; importantly, it must be possible to generate the annotations for this pretext task using the unlabeled data alone. For example, in a computer vision scenario, one could use a jigsaw puzzle task where patches are extracted from an image, scrambled randomly and then fed to a neural network that is trained to recover the original spatial ordering of the patches. If the network performs this task reasonably well, then it is conceivable that it has learned some of the structure of natural images, and that the trained network could be reused as a feature extractor or weight initialization on a smaller-scale supervised learning problem such as object recognition. Apart from facilitating the downstream task and/or reducing the number of necessary annotated examples, self-supervision can also uncover more general and robust features than those learned in a specialized supervised task. Therefore, given the potential benefits of SSL, it is of interest to find out whether it can be used to enhance the analysis of EEG.

To date, applications of SSL have focused on domains where plentiful annotated data is already available, such as computer vision and natural language processing. Particularly in computer vision, deep networks are often trained with fully supervised tasks (e.g., ImageNet pretraining). In this case, enough labeled data is available such that direct supervised learning on the downstream task is already in itself competitive. SSL has an even greater potential in domains where low-labeled data regimes are common, e.g. biosignal and EEG processing.

Self-supervision can bring improvements over standard supervised approaches on EEG. Generic representations of EEG can be learned with self-supervision and, in doing so, this can reduce the need for costly EEG annotations. Deep learning can be used as a processing tool for EEG and impact current practices in the field of EEG processing. Indeed, while deep learning is notoriously data-hungry, an overwhelming part of all neuroscience research happens in the low-labeled data regime, including EEG research: clinical studies with a few hundred subjects are often considered to be big data, while large-scale studies are much rarer and usually originate from research consortia. Therefore, it is to be expected that the performance reported by most deep learning-EEG studies—often in low-labeled data regimes—has so far remained limited and does not clearly outperform those of conventional approaches. By leveraging unlabeled data, SSL can effectively create a lot more examples, which could enable more successful applications of deep learning to EEG.

In some embodiments of the present disclosure, self-supervision is used as an approach to learning representations from EEG data. The present disclosure provides a detailed analysis of SSL tasks on multiple types of EEG recordings. Embodiments described herein can provide information on:
1. good SSL tasks that capture relevant structure in EEG data;
2. a comparison of SSL features to other unsupervised and supervised approaches in terms of downstream classification performance;
3. characteristics of the features learned by SSL; and
4. using SSL to capture physiologically- and clinically-relevant structure from unlabeled EEG.

Overview

According to some embodiments of the present disclosure, sets of windows can be sampled from training data. These sets can have their set representation (e.g., matching set, non-matching set, inconclusive) determined based on the time between the windows in the set (for some bio-signal tasks, windows close to one another likely share labels, while those further apart likely do not share labels). These same sets can have their set representation predicted based on the difference or similarity between the feature representations extracted from the windows using a neural network. The learning objective can be minimizing the difference between the determined set representations and the predicted set representations. The system can use labeled training data to label the unlabeled training data (e.g., comparing the representation of labelled data to unlabelled data to determine whether there may be label correspondence). The system can use learning processes or neural networks to minimize the number of classification conflicts between the set representations. Using the foregoing embodiment, one can train a neural network to classify bio-signal data based on training data that includes unlabeled windows.

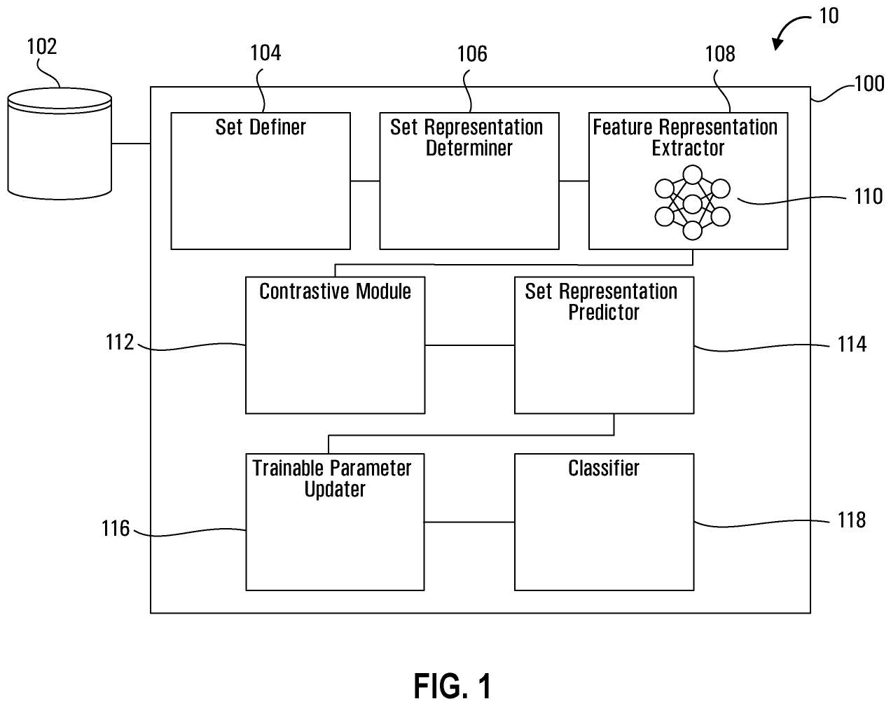

FIG. 1 illustrates a schematic diagram of a system capable of implementing SSL to train a neural network to label bio-signal data using labeled and unlabeled data, according to some embodiments. The system 10 can be a server with one or more hardware processors. System 10 can be configured to train a neural network to label bio-signal data from training data which is at least partially unlabeled. Partially labeled or unlabeled training bio-signal data can be stored on a memory 102 (including non-transitory memory). A set definer 104 can receive the training bio-signal data from memory 102. Set definer 104 can define sets within the training data. Set definer 104 can segment the training data into windows (e.g., segment into temporal intervals). Set definer 104 can define the sets of windows (e.g., pairs of windows, triplets of windows, sets with greater numbers, etc.). For one or more sets defined by set definer 104, a set representation determiner 106 can determine the set representation of a set which can denote the likely label correspondence (e.g., labels match, labels do not match, inconclusive). Label correspondence indicates whether the labels of a set likely correspond or not (even if the label itself is unknown). A feature representation extractor 108 can use an embedder neural network 110 to extract features from each of the time windows in the set. A contrastive module 112 can compare the extracted features of the windows in the set to determine their differences based on the features. A set representation predictor 114 can use the differences between the extracted features of the windows to predict the set representation of the set. One or more sets can have their set representations determined and predicted using this process. A trainable parameter updater 116 will then minimize the difference between the predicted and determined set representations by updating trainable parameters (e.g., trainable parameters of embedder neural network 110). A classifier 118 can then use any of the labeled training bio-signal data to apply labels to unlabeled training bio-signal data. According to some embodiments, the result of training using system 10 is a system capable of labeling bio-signal data based on features extracted using the embedder neural network 110. System 10 can learn bio-signal annotation from only partially annotated training data. In some embodiments, neural network 110 is capable of classifying (though not labeling) bio-signal data. In some embodiments, system 10 is further configured to train for a learning task using the labeled training bio-signal data.

In accordance with an aspect, there is provided a system 10 for training a neural network to classify bio-signal data by updating trainable parameters of the neural network. The system 10 has a memory 102 and a training computing apparatus 100 with at least one hardware processor. Memory 102 is configured to store training bio-signal data from one or more subjects. The training bio-signal data includes labeled training bio-signal data and unlabeled training bio-signal data. The training computing apparatus 100 is configured to receive the training bio-signal data from memory 102, define one or more sets of time windows within the training bio-signal data using set definer 104, each set including a first anchor window and a sampled window, for at least one set of the one or more sets, determine a determined set representation based in part on the relative position of the first anchor window and the sampled window using set representation determiner 106, extract a feature representation of the first anchor window and a feature representation of the sampled window using an embedder neural network 110 using feature representation extractor 108, aggregate the feature representations using contrastive module 112, and predict a predicted set representation using the aggregated feature representations using set representation predictor 114, update trainable parameters of the embedder neural network 110 to minimize a difference between the determined set representation of the at least one set and the predicted set representation of the at least one set using trainable parameter updater 116, and label the unlabeled training bio-signal data using a classifier 118, the labeled training bio-signal data, and embedder neural network 110. The set representation denotes likely label correspondence between the first anchor window and the sampled window.

In some embodiments memory 102 can store a training set of partially unlabeled training bio-signal data. In some embodiments, memory 102 can store bio-signal data that has been received from a bio-signal sensor. In some embodiments, memory 102 is replaced with one or more bio-signal sensors measuring bio-signals from a subject. In some embodiments the bio-signal data includes one or more different types of bio-signal data (EEG, EKG, PPG, EOG, breath, oxygenation, etc.). In some embodiments the training bio-signal data includes bio-signal measurements from one or more subjects taken during one or more monitoring sessions. In some embodiments, memory 102 can include additional non-bio-signal information such as demographic or personal information related to the subject, conditions of bio-signal collection (e.g., was the subject running or meditating during collection), or operator information (e.g., the identity of a practitioner that conducted the measurement or device with which measurement was conducted). The system 10 can access memory 102 to read data and store output data for subsequent access, and for access by authorized systems and components.

In some embodiments set definer 104 can temporally segment the data into windows. In some embodiments, the segmentation intervals can be optimized beforehand. In some embodiments, the segmentation intervals can be optimized as part of the training. In some embodiments, sets include pairs of windows. In some embodiments, sets include triplets of windows. In some embodiments, sets include many windows. In some embodiments, sets are defined with the windows chosen independently of one another (i.e., any window can be included in a set with any other window). In some embodiments, set definer 104 can define sets based in part on the proximity of windows (e.g., some triplets can include a first window, a second window proximate to the first window, and a third window). In some embodiments, the windows included in the sets may be from the same bio-signal measuring session. In some embodiments, the windows included in the set may originate from the same subject during different bio-signal measuring sessions. In some embodiments, the windows included in the set may originate from different subjects, but with one or more characteristics in common (e.g., subject performing the same activity, measured at the same location, measured by the same practitioner, etc.). One skilled in the art would readily understand that set definer 104 can be configured to include combinations of the above rules when preparing sets (e.g., a triplet can include a first window from a first session, a second window from the first session proximate to the first window, and a third window from a different session from a different subject).

Set representation determiner 106 can use the temporal proximity of the windows in the set to determine that set's determined set representation. In some embodiments, set representation 106 can use a window in the set as an anchor window and define a positive and/or negative context region surrounding the anchor window. In these embodiments, a second window falling within the positive context region (i.e., closer to the anchor window) is determined to be a matching set (i.e., the windows likely share a label even if that label is not yet known). In these embodiments, a second window falling within the negative context region (i.e., farther from the anchor window) is determined to be a non-matching set (i.e., the windows likely have different labels even if those labels are not yet known). In these embodiments, a second window falling within neither the positive or negative context region could produce an inconclusive set representation and may be discarded from the training process.

In some embodiments, set definer 104 defines triplets with a first anchor window, a second anchor window temporally proximate to the first, and a third window. In such embodiments, set representation determiner 106 can determine the determined set representation by determining the temporal order of the windows. In such embodiments if the third window falls between the anchor windows, then the set is considered to be a matching set. In such embodiments, if the third window falls outsides the region between the two anchor windows, then the set can be considered non-matching or inconclusive set. In such embodiments, the region between the first and second anchor windows can be considered a positive context region. The region outside the two anchor windows can be defined wholly or partly as a negative context region (e.g., a third window falling outside the anchor windows, but immediately adjacent to one of said anchor windows may not necessarily fall into the negative context region, but a window further away might fall within the negative context region).

In some embodiments, the set representation is determined indirectly by set definer 104. For example, set definer 104 can select a series of consecutive anchor windows and a series of consecutive sampled windows immediately adjacent to the series of anchor windows to be in the positive context region (i.e., the labels of the anchor windows and the sampled windows likely match). This set can also include a set of negative context windows selected randomly from the training data. In such embodiments, set representation determiner 106 determines that windows from the series of consecutive sampled windows match the anchor windows, while the negative context windows do not necessarily match the anchor windows.

Feature representation extractor 108 can extract feature representations from the windows using embedder neural network 110. Embedder neural network 110 can be trained by adjusting the trainable parameters of the neural network to minimize a difference between the determined set representations and the predicted set representations. Embedder neural network 110 can be used to process data of different modalities. For example, in some embodiments, the bio-signal data may include EEG data and another type of data such as EKG data. Embedder neural network 110 can be configured to process the EEG and EKG data from the same window differently to extract the feature representations.

Contrastive module 112 can determine the difference between the features extracted from the windows. In some embodiments, contrastive module 112 can be a neural network trained to compare the feature representations of windows. In such embodiments, the contrastive module can include trainable parameters that can be updated to minimize the difference between determined set representations and predicted set representations. In some embodiments, contrastive module 112 compares a pair of windows. In some embodiments, contrastive module 112 compares a triplet of windows. In some embodiments, contrastive module 112 compares the feature representations of a series of anchor windows, one or more windows in a series of sampled windows, and one or more windows in a set of negative sample windows. In such embodiments, contrastive module 112 can aggregate the feature representations of each of the series of anchor windows and compare the aggregated anchor feature representations to the feature representations of the sampled windows and the negative context windows. The role of contrastive module 112 is to highlight the differences between the input representations to make the classification of matching/non-matching set easier. In some embodiments, contrastive module 112 concatenates its input representations and lets classifier 118 handle all the required processing.

Set representation predictor 114 can use the difference between the feature representations determined by contrastive module 112 to predict a set representation of the set of windows. In some embodiments, set representation predictor 114 determines whether a pair of windows are a matching set or a non-matching set. In some embodiments, set representation predictor 114 can determine if a set is inconclusive (and possibly discard it). In some embodiments, set representation predictor 114 can determine if a third window in a triplet of windows matches two anchor windows. In some embodiments, set representation predictor 114 can determine which of at least two windows (one or more sampled windows and one or more negative context windows) belongs to a series of consecutive sampled windows immediately adjacent to a series of consecutive anchor windows.

The functions carried out by set representation determiner 106, feature representation extractor 108, contrastive module 112, and set representation predictor 114 can be carried out for one or more sets defined by set definer 104. The set representation can be determined by set determiner 106 before, after, or at the same time as the functions carried out by feature representation extractor 108, contrastive module 112, or set representation predictor 114.

Trainable parameter updater 116 can update the trainable parameters of at least the embedder neural network 110. In some embodiments, trainable parameter updater 116 can determine the loss function using the determined set representations determined by set representation determiner 106 and the predicted set representations predicted by set representation predictor 114. The loss function can be minimized to maximize the number of predicted set representations that match the corresponding determined set representations. In some embodiments, the loss function uses the binary logistic loss on the predictions of the set representation predictor 114. In some embodiments, the loss function is a categorical cross-entropy loss.

In some embodiments, classifier 118 can label the unlabeled training bio-signal data using the labeled training bio-signal data and the predicted set representations. In some embodiments, classifier 118 can include a classifier neural network that has trainable parameters. In such embodiments, classifier 118 may label the unlabeled bio-signal data before trainable parameter updater 116 updates the trainable parameters. In some embodiments, classifier 118 may only label some of the unlabeled bio-signal data. In some embodiments, classifier 118 may label all of the unlabeled bio-signal data. In some embodiments, classifier 118 may provide labels for labeled bio-signal data (e.g., to indicate possible errors in the initial labeling of the training bio-signal data). In some embodiments, the labels generated can be used in downstream learning and/or predictive tasks.

In some embodiments, training computing apparatus 100 is further configured to store the training data labelled using classifier 118 in memory 102. In some embodiments, training computing apparatus 100 is further configured to transmit the training bio-signal data labelled using classifier 118 to another computing apparatus.

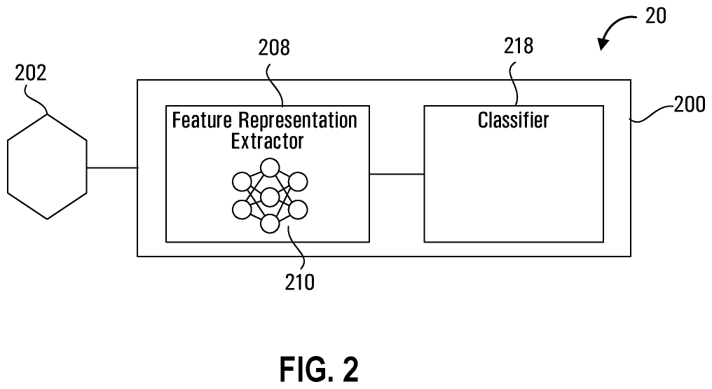

FIG. 2 illustrates a schematic diagram of a system using a neural network to label bio-signal data trained with SSL using labeled and unlabeled data, according to some embodiments. System 20 includes a bio-signal sensor 202 and a classifying computing apparatus 200 with at least one processor. Classifying computing apparatus 200 can receive the embedder neural network 110 trained in system 10 for use as an embedder neural network 210 for use in the feature representation extractor 208. Classifying computing apparatus 200 is configured to receive bio-signal data from bio-signal sensor 202. Feature extractor 208 can extract features from the window using embedder neural network 210. Classifier 218 can label the unlabeled bio-signal data using the embedder neural network 210.

Some embodiments further include a bio-signal sensor 202 and a classifying computing apparatus 200 with at least one processor and memory. Bio-signal sensor 202 is configured to receive user bio-signal data from a user. Classifying computing apparatus 200 is configured to receive the embedder neural network from the training computing apparatus 100, receive the user bio-signal data from bio-signal sensor 202, and label the user bio-signal data using the embedder neural network 210 and the classifier 218.

Components of system 20 can operate similarly to corresponding components of system 10 used to train the neural networks implemented in system 20. In some embodiments, embedder neural network 210 can be trained using system 10 (as embedder neural network 110) before being implemented in system 20. In some embodiments, classifier 218 can be trained using system 10 (as classifier 118) before being implemented in system 20.

In some embodiments, a memory (not shown) can be configured to provide the bio-signal data to classifying computing apparatus 200. In some embodiments, this memory can provide bio-signal data to system 200 in addition to or instead of the bio-signal data provided by bio-signal sensor 202. In some embodiments, bio-signal sensor 202 is absent.

System 20 can be configured to use a neural network to label unlabeled bio-signal data. Bio-signal data can be measured using bio-signal sensor 202. Feature representation extractor 208 can use an embedder neural network 210 to extract features from the time windows. Classifier 218 can then label the bio-signal data. According to some embodiments, the result of using system 20 is at least partially labeled bio-signal data.

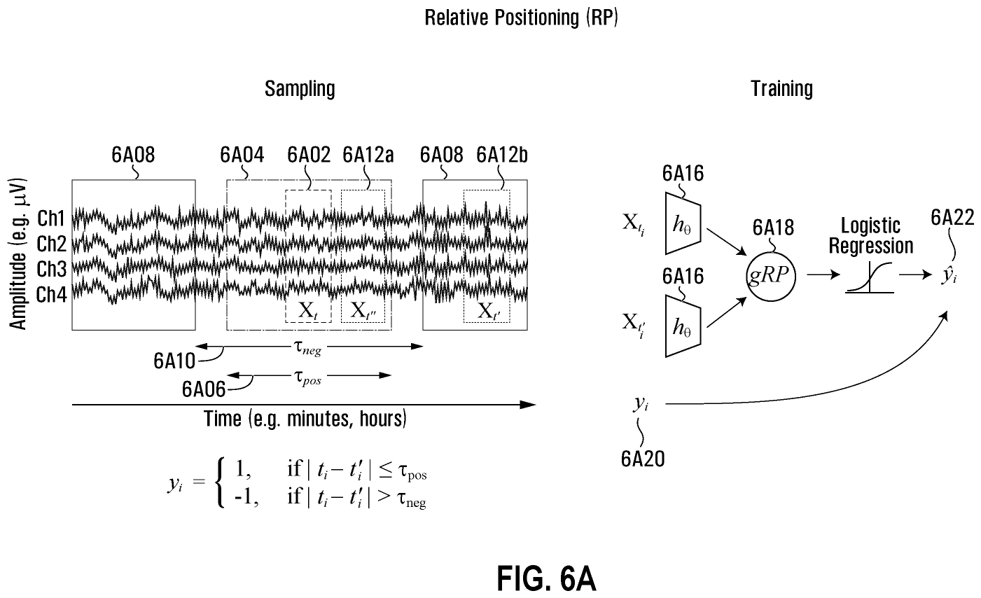

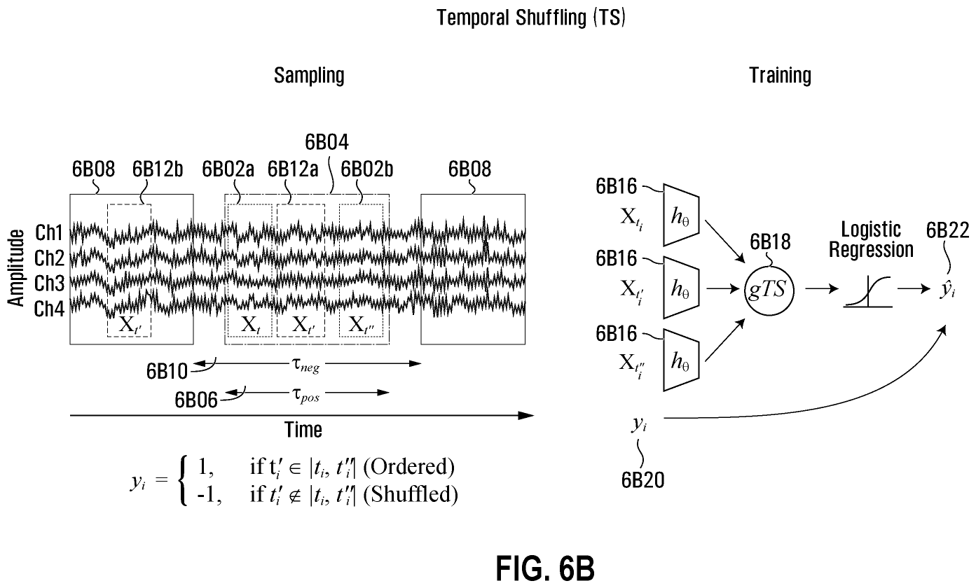

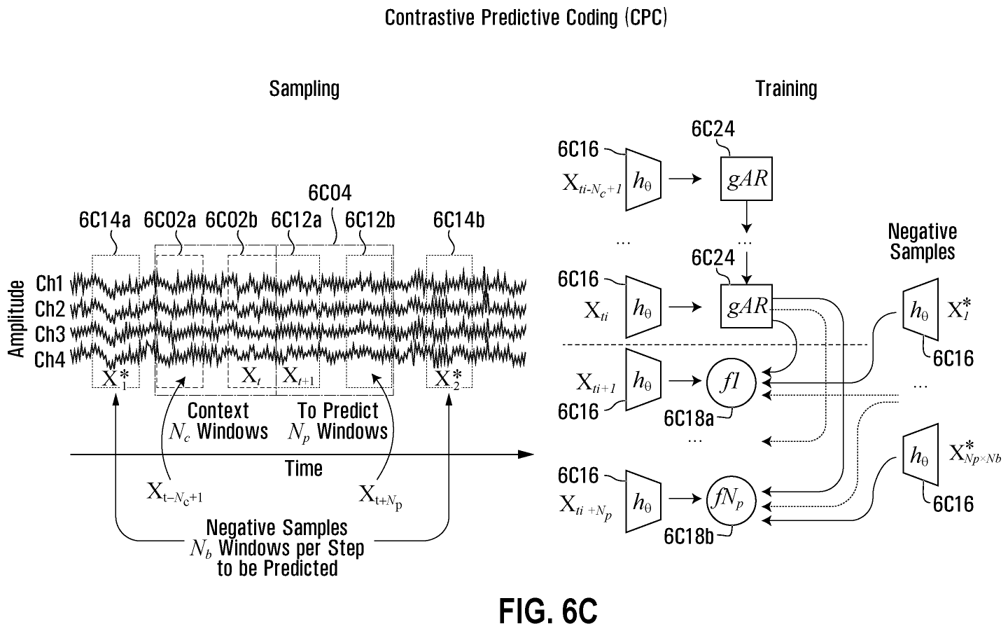

Referring to FIG. 6A, in some embodiments, the sets can be pairs of windows including an anchor window 6A02, and a sampled window 6A12 (a or b). In these embodiments the set representation can be determined by defining a positive context region 6A04 surrounding anchor window 6A02 using positive context distance 6A06. A negative context region 6A08 can optionally be defined around anchor window 6A02 using negative context distance 6A10. The determined set representation 6A20 can be determined by determining where sampled window 6A12 falls. For example, if sampled window 6A12 falls within positive context region 6A04 (e.g., sampled window 6A12a) then determined set representation 6A20 is a match. If, however, sampled window 6A12 falls within negative context region 6A08 (e.g., sampled window 6A12b) then determined set representation 6A20 is a non-match. In some embodiments, if 6A12 falls into neither positive context region 6A04 or negative context region 6A08, then determined set representation 6A20 can be inconclusive and can be removed from training.

In some embodiments, embedder neural network 6A16 can extract feature representations of the pair of windows. A contrastive module 6A18 can aggregate the feature representations generated by embedders 6A16 to predict a predicted set representation 6A22. The system can learn to reduce the difference between determined set representation 6A20 and predicted set representation 6A22 by adjusting trainable parameters (e.g., those in embedder neural network 6A16).

Referring more specifically to embedder neural network 6A16, it can be configured to process different modalities of data. For example, in some embodiments, the bio-signal data may include EEG data and another type of data such as EKG data. Embedder neural network 6A16 can be configured to process the EEG and EKG data differently to extract the feature representations.

In some embodiments, the one or more sets of time windows include one or more pairs of time windows, the at least one set of the one or more sets include at least one pair of the one or more pairs, and the training computing apparatus determines the determined set representation 6A20 based in part on the relative position of the first anchor window 6A02 and the sampled window 6A12 by defining a positive context region 6A04 and negative context region 6A08 surrounding the first anchor window 6A02, and determining if the sampled window 6A12 is within the positive context region 6A04 or negative context region 6A08.

In some embodiments, the determined set representation is based in part on the relative position of the first anchor window 6A02 and the sampled window 6A12 further includes rejecting the at least one pair if the sampled window 6A12 is not within the positive context region 6A04 or the negative context region 6A08.

Referring to FIG. 6B, in some embodiments, the sets can be triplets of windows including a first anchor window 6B02a, a second anchor window 6B02b and a sampled window 6612 (a or b). The second anchor window 6B02b can be defined as being within a positive context distance 6606 of first anchor window 6B02a. In these embodiments the set representation can be determined by defining a positive context region 6604 between anchor windows 6B02a and 6B02b. A negative context region 6608 can optionally be defined around anchor window 6602 using negative context distance 6610. In some embodiments, the negative context region 6608 is defined as being a distance of negative context distance 6610 from the mid-point between anchor windows 6B02a and 6B02b. The determined set representation 6620 can be determined by determining the order of anchor windows 6B02a and 6B02b, and sampled window 6612. For example, if sampled window 6612 falls within anchor windows 6B02a and 6B02b (e.g., sampled window 6B12a) then determined set representation 6620 is a match. If, however, sampled window 6612 falls into negative context region 6608, then determined set representation 6620 can be non-match.

In some embodiments, embedder neural network 6616 can extract feature representations of the triplet of windows. A contrastive module 6618 can aggregate the feature representations generated by embedders 6616 to predict a predicted set representation 6622. The system can learn to reduce the difference between determined set representation 6620 and predicted set representation 6622 by adjusting trainable parameters (e.g., those in embedder neural network 6616).

In some embodiments, the one or more sets of time windows include one or more triplets of time windows, each triplet further including a second anchor window 6B02b, wherein the second anchor window 6B02b is within a positive context region 6604 surrounding the first anchor window 6B02a, the at least one set of the one or more sets includes at least one triplet of the one or more triplets, the training computing apparatus determines the determined set representation 6620 based in part on the relative position of the first anchor window 6B02a and the sampled window 6612 by determining a temporal order of the first anchor window 6B02a, the sampled window 6612, and the second anchor window 6B02b, and the extract the feature representation of the first anchor window 6B02a and a feature representation of the sampled window 6612 using an embedder neural network further includes extracting a feature representation of the second anchor window 6B02b.

In some embodiments, define the one or more triplets of time windows within the training bio-signal data further includes for one triplet of the one or more triplets, a mirror of the one triplet is included in the one or more triplets.

Referring to FIG. 6C, in some embodiments, the sets can include a series of anchor windows 6C02 (including 6C02a and 6C02b), a series of sampled windows 6C12 (including 6C12a and 6C12b), and a set of negative context windows 6C14 (including 6C14a and 6C14b). The series of consecutive sampled windows 6C12 can be immediately adjacent to the series of consecutive anchor windows 6C02 such that all of the sampled windows 6C12 are determined to match all of the series of anchor windows 6C02. The set of negative sample windows 6C14 can be sampled at random from the bio-signal data such that they do not match the series of consecutive anchor windows 6C02.

In these embodiments, embedder neural network 6C16 can extract the feature representations of the series of consecutive anchor windows 6C02. The extracted feature representations of the anchor windows 6C02 can be embedded using an autoregressive embedder 6C24. Embedder neural network 6C16 can extract a feature representation of a given sampled window 6C12a. Embedder neural network 6C16 can extract a feature representation of a given negative sample window 6C14a. The contrastive module 6C18a can aggregate the embedded feature representations of the anchor windows 6C02, the feature representation of the given sampled window 6C12a, and the feature representation of the given negative sample window 6C14a to predict which window of the given sampled window 6C12a and the given negative sample window 6C14a is the given sampled window 6C12a (i.e., matches the anchor windows 6C02).

In some embodiments, embedder neural network 6C16 can extract a feature representation of another given sampled window 6C12b. Embedder neural network 6C16 can extract a feature representation of another given negative sample window 6C14b. The contrastive module 6C18b can aggregate the embedded feature representations of the anchor windows 6C02, the feature representation of the other given sampled window 6C12b, and the feature representation of the given other negative sample window 6C14b to predict which window of the other given sampled window 6C12b and the other given negative sample window 6C14b is the another given sampled window 6C12b (i.e., matches the anchor windows 6C02). In some embodiments, further another given sampled windows can be compared to further another given negative sample windows.

In some embodiments, one given sampled window can be compared to one or more given negative sampled windows.

In some embodiments, the first anchor window includes a series of consecutive anchor windows 6C02, the sampled window includes a series of consecutive sampled windows 6C12, wherein the series of consecutive sampled windows 6C12 is adjacent to the series of consecutive anchor windows 6C02, the set further includes a set of negative sample windows 6C14, and the training computing apparatus determines the determined set representation based in part on the relative position of the first anchor window and the sampled window by determining that a given sampled window 6C12a is in the series of sampled windows 6C12. In such embodiments, the extract the feature representation of the first anchor window and a feature representation of the sampled window using an embedder neural network includes extracting a feature representation of each anchor window of the series of consecutive anchor windows 6C02, extracting a feature representation of each sampled window of the series of consecutive sampled windows 6C12, and extracting a feature representation of each negative sample window of the set of negative sample windows 6C14. In such embodiments, the aggregate the feature representations includes embedding the feature representation of each anchor window of the series of anchor windows using an autoregressive embedder 6C24, and aggregating the embedded anchor series, a given feature representation of a given sampled window of the series of sampled windows 6C12, and one or more given feature representations of one or more given negative sample windows 6C14 of the set of negative sample windows. In such embodiments, the predict the predicted set representation includes predicting which of the given feature representations corresponds to the given feature representation of the sampled window of the series of sampled windows 6C12.

Referring to FIG. 1, in some embodiments, embedder neural network 110 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the label the user bio-signal data includes determining a brain state of the user. In some embodiments, the label the user bio-signal data includes determining a sleep state of the user. In some embodiments, the label the user bio-signal data includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

In some embodiments, training computing apparatus 100 includes a server having at least one hardware processor. In some embodiments, training computing apparatus 100 can be housed within a server permitting system 10 to update its training. In some embodiments, said server can push or otherwise make accessible the latest trained system for one or more classifying computing apparatuses 200.

In some embodiments, training computing apparatus 100 includes a server configured to upload embedder neural network 110 and classifier 118 to the classifier computing apparatus 200.

In some embodiments, training computing apparatus 100 includes classifying computing apparatus 200.

In some embodiments, classifier 118 includes a classifier neural network, and the update trainable parameters includes updating trainable parameters of the classifier neural network. In such embodiments, system 10 can train embedder neural network 110 and the classifier neural network in an end-to-end manner. In some embodiments, classifying computing apparatus 200 can be configured to receive and implement trained embedder neural network 110 and the classifier neural network.

In some embodiments, contrastive module 112 includes a contrastive neural network, and the update trainable parameters further includes updating trainable parameters of the contrastive neural network. In such embodiments, system 10 can train embedder neural network 110 and the contrastive neural network in an end-to-end manner. In some embodiments, classifying computing apparatus 200 can be configured to receive and implement trained embedder neural network 110 and the contrastive neural network.

In some embodiments, classifier 118 labels unlabeled bio-signal data based in part on a personal characteristic of the subject corresponding to the training bio-signal data, and the classifying the user bio-signal data is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

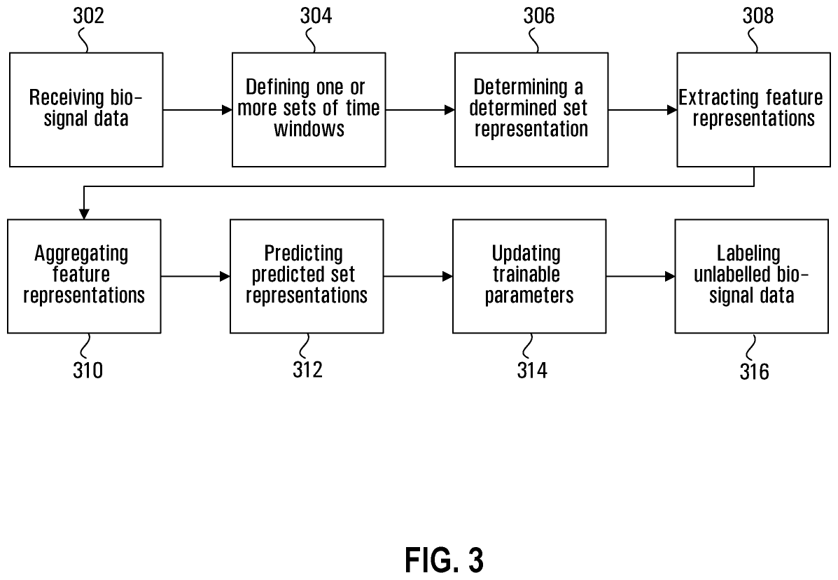

FIG. 3 illustrates a flowchart showing example operation of the SSL training operation implemented in FIG. 1, according to some embodiments.

In accordance with another aspect, there is provided a method for training a neural network to classify bio-signal data by updating trainable parameters of the neural network. The method includes receiving training bio-signal data from one or more subjects including labeled training bio-signal data and unlabeled training bio-signal data (302), defining one or more sets of time windows within the training bio-signal data (304), each set including a first anchor window and a sampled window, for at least one set of the one or more sets, determining a determined set representation based in part on the relative position of the first anchor window and the sampled window (306), extracting a feature representation of the first anchor window and a feature representation of the sampled window using an embedder neural network (308), aggregating the feature representations using a contrastive module (310), predicting a predicted set representation using the aggregated feature representations (312), updating trainable parameters of the embedder neural network to minimize a difference between the determined set representation of the at least one set and the predicted set representation of the at least one set (314), and labeling the unlabeled training bio-signal data using a classifier, the labeled training bio-signal data, and the embedder neural network (316). The set representation denotes likely label correspondence between the first anchor window and the sampled window.

Some embodiments further include receiving user bio-signal data from a user using a bio-signal sensor, and labeling the user bio-signal data using the embedder neural network and the classifier.

Some embodiments further comprising storing the training data labelled using a classifier in a memory. Some embodiments further comprising transmitting the training bio-signal data labelled using the classifier to another computing apparatus.

In some embodiments, the one or more sets of time windows are one or more pairs of time windows, the at least one set of the one or more sets are at least one pair of the one or more pairs, the determining a determined set representation based in part on the relative position of the first anchor window and the sampled window (306) includes defining a positive context region and negative context region surrounding the first anchor window, and determining if the sampled window is within the positive context region or negative context region.

In some embodiments, the determining a determined set representation based in part on the relative position of the first anchor window and the sampled window (306) further includes rejecting the at least one pair if the sampled window is not within the positive context region or the negative context region.

In some embodiments, the one or more sets of time windows are one or more triplets of time windows, each triplet further includes a second anchor window, wherein the second anchor window is within a positive context region surrounding the first anchor window, the at least one set of the one or more sets is at least one triplet of the one or more triplets, the determining a determined set representation based in part on the relative position of the first anchor window and the sampled window (306) includes determining a temporal order of the first anchor window, the sampled window, and the second anchored window, and the extract a feature representation of the first anchor window and a feature representation of the sampled window using an embedder neural network (308) further includes extracting a feature representation of the second anchor window.

In some embodiments, defining one or more triplets of time windows within the training bio-signal data further (304) includes for one triplet of the one or more triplets, a mirror of the one triplet is included in the one or more triplets.

In some embodiments, the first anchor window includes a series of consecutive anchor windows, the sampled window includes a series of consecutive sampled windows, wherein the series of consecutive sampled windows is adjacent to the series of consecutive anchor windows, the set further includes a set of negative sample windows, the determining a determined set representation based in part on the relative position of the first anchor window and the sampled window (306) includes determining that a given sampled window is in the series of sampled windows. In such embodiments, the extracting a feature representation of the first anchor window and a feature representation of the sampled window using an embedder neural network (308) further includes extracting a feature representation of each anchor window of the series of consecutive anchor windows, extracting a feature representation of each sampled window of the series of consecutive sampled windows, and extracting a feature representation of each negative sample window of the set of negative sample windows. In such embodiments, the aggregating the feature representations (310) includes embedding the feature representation of each anchor window of the series of anchor windows using an autoregressive embedder, and aggregating the embedded anchor series, a given feature representation of a given sampled window of the series of sampled windows, and one or more given feature representations of one or more given negative sample windows of the set of negative sample windows. In such embodiments, the predicting a predicted set representation (312) includes predicting which of the given feature representations corresponds to the given feature representation of the sampled window of the series of sampled windows.

In some embodiments, the embedder neural network includes one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the labeling the user bio-signal data (316) includes determining a brain state of the user. In some embodiments, the labeling the user bio-signal data (316) includes determining a sleep state of the user. In some embodiments, the labeling the user bio-signal data (316) includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

Some embodiments further including uploading the embedder neural network to a server. In some embodiments, said server can push or otherwise make accessible the latest trained system for one or more classifying computing apparatuses.

In some embodiments, the contrastive module includes a contrastive neural network, and the updating trainable parameters further (314) includes updating trainable parameters of the contrastive neural network. In such embodiments, the method can train the embedder neural network and the contrastive neural network in an end-to-end manner.

In some embodiments, the classifier includes a classifier neural network, and the update trainable parameters (314) includes updating trainable parameters of the classifier neural network. In such embodiments, the method can train embedder neural network and the classifier neural network in an end-to-end manner.

In some embodiments, the classifier labels unlabeled bio-signal data based in part on a personal characteristic of the subject corresponding to the training bio-signal data, and the classifying the user bio-signal data (316) is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

Referring to FIG. 1, in accordance with another aspect, there is provided a system for training a neural network to classify bio-signal data by updating trainable parameters of the neural network. The system includes a memory 102 and a training computing apparatus 100. Memory 102 is configured to store training bio-signal data from one or more subjects. The training bio-signal data includes labeled training bio-signal data and unlabeled training bio-signal data. Training computing apparatus 100 is configured to receive the training bio-signal data from memory 102, define one or more pairs of time windows within the training bio-signal data using a set definer 104, each pair including an anchor window and a sampled window, for at least one pair of the one or more pairs, define a positive context region and negative context region surrounding the anchor window using set representation determiner 106, determine a determined pair representation based on whether the sampled window is within the positive context region or negative context region using set representation determiner 106, extract a feature representation of the anchor window and a feature representation of the sampled window using embedder neural network 110 using feature representation extractor 108, aggregate the feature representations using contrastive module 112, and predict a predicted pair representation using the aggregated feature representations using set representation predictor 114, update trainable parameters of embedder neural network 110 to minimize a difference between the determined pair representation of the at least one pair and the predicted pair representation of the at least one pair using trainable parameter updater 116, and label the unlabeled training bio-signal data using classifier 118, the labeled training bio-signal data, and embedder neural network 110. The pair representation denotes likely label correspondence between the anchor window and the sampled window.

Some embodiments further include a bio-signal sensor 202 and a classifying computing apparatus 200. Bio-signal sensor 202 is configured to receive user bio-signal data from a user. Classifying computing apparatus 200 is configured to receive the embedder neural network from the training computing apparatus 100, receive the user bio-signal data from bio-signal sensor 202, and label the user bio-signal data using the embedder neural network 210 and the classifier 218.

In some embodiments, training computing apparatus 100 is further configured to store the training data labelled using classifier 118 in memory 102. In some embodiments, training computing apparatus 100 is further configured to transmit the training bio-signal data labelled using classifier 118 to another computing apparatus.

In some embodiments, the determined set representation is based in part on the relative position of the anchor window 6A02 and the sampled window 6A12 further includes rejecting the at least one pair if the sampled window 6A12 is not within the positive context region 6A04 or the negative context region 6A08.

In some embodiments, embedder neural network 110 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the label the user bio-signal data includes determining a brain state of the user. In some embodiments, the label the user bio-signal data includes determining a sleep state of the user. In some embodiments, the label the user bio-signal data includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

In some embodiments, training computing apparatus 100 includes a server having at least one hardware processor. In some embodiments, training computing apparatus 100 can be housed within a server permitting system 10 to update its training. In some embodiments, said server can push or otherwise make accessible the latest trained system for one or more classifying computing apparatuses 200.

In some embodiments, training computing apparatus 100 includes a server configured to upload embedder neural network 110 and classifier 118 to the classifier computing apparatus 200.

In some embodiments, training computing apparatus 100 includes classifying computing apparatus 200.

In some embodiments, classifier 118 includes a classifier neural network, and the update trainable parameters includes updating trainable parameters of the classifier neural network. In such embodiments, system 10 can train embedder neural network 110 and the classifier neural network in an end-to-end manner. In some embodiments, classifying computing apparatus 200 can be configured to receive and implement trained embedder neural network 110 and the classifier neural network.

In some embodiments, contrastive module 112 includes a contrastive neural network, and the update trainable parameters further includes updating trainable parameters of the contrastive neural network. In such embodiments, system 10 can train embedder neural network 110 and the contrastive neural network in an end-to-end manner. In some embodiments, classifying computing apparatus 200 can be configured to receive and implement trained embedder neural network 110 and the contrastive neural network.

In some embodiments, classifier 118 labels unlabeled bio-signal data based in part on a personal characteristic of the subject corresponding to the training bio-signal data, and the classifying the user bio-signal data is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

In accordance with another aspect, there is provided a system for training a neural network to classify bio-signal data. The system includes a memory 102 and a training computing apparatus 100. Memory 102 is configured to store training bio-signal data from one or more subjects. The training bio-signal data includes labeled training bio-signal data and unlabeled training bio-signal data. Training computing apparatus 100 is configured to receive the training bio-signal data from memory 102, define one or more triplets of time windows within the training bio-signal data using set definer 104, each triplet including a first anchor window, a second anchor window, and a sampled window, for at least one triplet of the one or more triplets, determine a determined triplet representation based in part on the temporal order of the first anchor window, the second anchor window, and the sampled window using set representation determiner 106, extract a feature representation of the first anchor window, a feature representation of the second anchor window, and a feature representation of the sampled window using an embedder neural network 110 using feature representation extractor 108, aggregate the feature representations using contrastive module 112, and predict a predicted triplet representation using the aggregated feature representations using set representation predictor 114, update trainable parameters of the embedder neural network to minimize a difference between the determined triplet representation of the at least one triplet and the predicted triplet representation of the at least one triplet using trainable parameter updater 116, and label the unlabeled training bio-signal data using classifier 118, the labeled training bio-signal data, and embedder neural network 110. The second anchor window is within a positive context region surrounding the first anchor window. The triplet representation denotes likely label correspondence between the first anchor window and the sampled window.

Some embodiments further include a bio-signal sensor 202 and a classifying computing apparatus 200. Bio-signal sensor 202 is configured to receive user bio-signal data from a user. Classifying computing apparatus 200 is configured to receive the embedder neural network from the training computing apparatus 100, receive the user bio-signal data from bio-signal sensor 202, and label the user bio-signal data using the embedder neural network 210 and the classifier 218.

In some embodiments, training computing apparatus 100 is further configured to store the training data labelled using classifier 118 in memory 102. In some embodiments, training computing apparatus 100 is further configured to transmit the training bio-signal data labelled using classifier 118 to another computing apparatus.

In some embodiments, the define the one or more triplets of time windows within the training bio-signal data further includes for one triplet of the one or more triplets, a mirror of the one triplet is included in the one or more triplets.

In some embodiments, embedder neural network 110 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the label the user bio-signal data includes determining a brain state of the user. In some embodiments, the label the user bio-signal data includes determining a sleep state of the user. In some embodiments, the label the user bio-signal data includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

In some embodiments, training computing apparatus 100 includes a server having at least one hardware processor. In some embodiments, training computing apparatus 100 can be housed within a server permitting system 10 to update its training. In some embodiments, said server can push or otherwise make accessible the latest trained system for one or more classifying computing apparatuses 200.

In some embodiments, training computing apparatus 100 includes a server configured to upload embedder neural network 110 and classifier 118 to the classifier computing apparatus 200.

In some embodiments, training computing apparatus 100 includes classifying computing apparatus 200.

In some embodiments, classifier 118 includes a classifier neural network, and the update trainable parameters includes updating trainable parameters of the classifier neural network. In such embodiments, system 10 can train embedder neural network 110 and the classifier neural network in an end-to-end manner. In some embodiments, classifying computing apparatus 200 can be configured to receive and implement trained embedder neural network 110 and the classifier neural network.

In some embodiments, contrastive module 112 includes a contrastive neural network, and the update trainable parameters further includes updating trainable parameters of the contrastive neural network. In such embodiments, system 10 can train embedder neural network 110 and the contrastive neural network in an end-to-end manner. In some embodiments, classifying computing apparatus 200 can be configured to receive and implement trained embedder neural network 110 and the contrastive neural network.

In some embodiments, classifier 118 labels unlabeled bio-signal data based in part on a personal characteristic of the subject corresponding to the training bio-signal data, and the classifying the user bio-signal data is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

In accordance with another aspect, there is provided a system for training a neural network to classify bio-signal data by updating trainable parameters of the neural network. The system includes a memory 102 and a training computing apparatus 100. Memory 102 is configured to store training bio-signal data from one or more subjects. The training bio-signal data includes labeled training bio-signal data and unlabeled training bio-signal data. Training computing apparatus 100 is configured to receive the training bio-signal data from memory 102, define one or more sets of time windows within the training bio-signal data using set definer 104, each set including a series of consecutive anchor windows, a series of consecutive sampled windows, and a set of negative sample windows, for at least one set of the one or more sets, extract a feature representation of each anchor window of the series of consecutive anchor windows, a feature representation of each sampled window of the series of consecutive sampled windows, and a feature representation of each negative sample window of the set of negative sample windows using embedder neural network 110 using feature representation extractor 108, embed the feature representation of each anchor window of the series of anchor windows using an autoregressive embedder, aggregate the embedded feature representation of each anchor window, a given feature representation of a given sampled window, and one or more given feature representations of one or more given negative sample windows using contrastive module 112, and predict which of the given sampled window and the one or more given negative sample windows is the given sampled window based on the aggregated feature representations using set representation predictor 114, update trainable parameters of embedder neural network 110 to minimize predictions that predict the one or more given negative sample windows is the given sampled window using trainable parameter updater 116, and label the unlabeled training bio-signal data using classifier 118, the labeled training bio-signal data, and embedder neural network 110. The series of consecutive sampled windows is adjacent to the series of consecutive anchor windows.

Some embodiments further include a bio-signal sensor 202 and a classifying computing apparatus 200. Bio-signal sensor 202 is configured to receive user bio-signal data from a user. Classifying computing apparatus 200 is configured to receive the embedder neural network from the training computing apparatus 100, receive the user bio-signal data from bio-signal sensor 202, and label the user bio-signal data using the embedder neural network 210 and the classifier 218.

In some embodiments, training computing apparatus 100 is further configured to store the training data labelled using classifier 118 in memory 102. In some embodiments, training computing apparatus 100 is further configured to transmit the training bio-signal data labelled using classifier 118 to another computing apparatus.

In some embodiments, embedder neural network 110 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the label the user bio-signal data includes determining a brain state of the user. In some embodiments, the label the user bio-signal data includes determining a sleep state of the user. In some embodiments, the label the user bio-signal data includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

In some embodiments, training computing apparatus 100 includes a server having at least one hardware processor. In some embodiments, training computing apparatus 100 can be housed within a server permitting system 10 to update its training. In some embodiments, said server can push or otherwise make accessible the latest trained system for one or more classifying computing apparatuses 200.

In some embodiments, training computing apparatus 100 includes a server configured to upload the embedder neural network 110 and classifier 118 to the classifier computing apparatus 200.

In some embodiments, training computing apparatus 100 includes classifying computing apparatus 200.

In some embodiments, classifier 118 includes a classifier neural network, and the update trainable parameters includes updating trainable parameters of the classifier neural network. In such embodiments, system 10 can train embedder neural network 110 and the classifier neural network in an end-to-end manner. In some embodiments, classifying computing apparatus 200 can be configured to receive and implement trained embedder neural network 110 and the classifier neural network.

In some embodiments, contrastive module 112 includes a contrastive neural network, and the update trainable parameters further includes updating trainable parameters of the contrastive neural network. In such embodiments, system 10 can train embedder neural network 110 and the contrastive neural network in an end-to-end manner. In some embodiments, classifying computing apparatus 200 can be configured to receive and implement trained embedder neural network 110 and the contrastive neural network.

In some embodiments, classifier 118 labels unlabeled bio-signal data based in part on a personal characteristic of the subject corresponding to the training bio-signal data, and the classifying the user bio-signal data is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

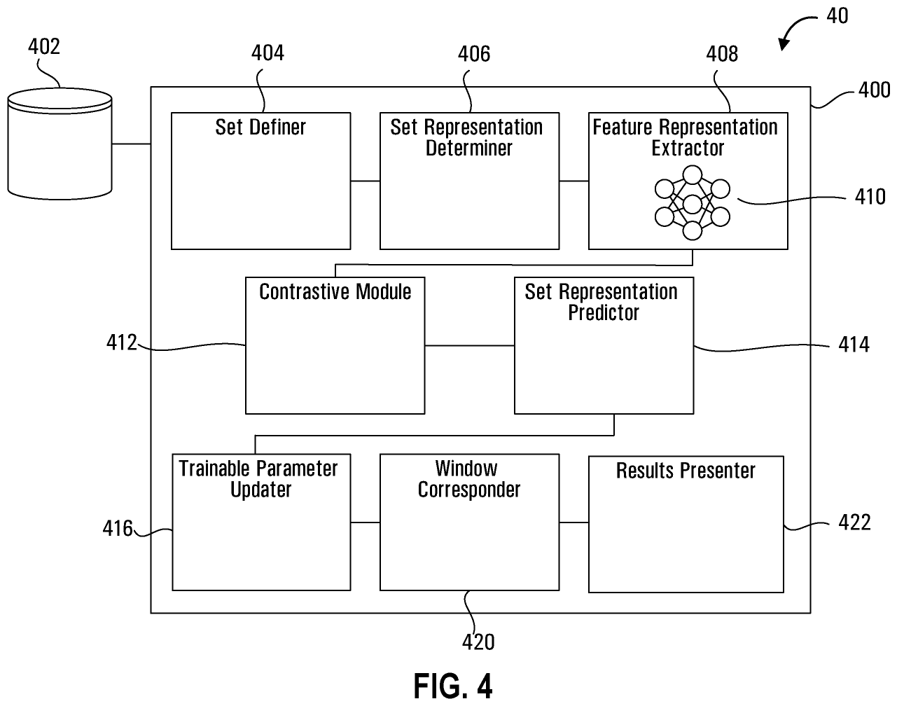

FIG. 4 illustrates a schematic diagram of a system capable of implementing SSL to train a neural network to group bio-signal data using unlabeled data, according to some embodiments.

System 40 illustrates an exemplary system capable of being configured to group windows in unlabeled data together and present those findings to a practitioner. System 40 includes a memory 402 and a computing apparatus 400. Computing apparatus 400 is configured to receive bio-signal data from memory 402. Set definer 404 can define sets within the bio-signal data. Set representation determiner 406 can determine the set representations based on the proximity of windows within the set. Feature extractor 408 can extract features from the window using embedder neural network 410. Contrastive module 412 can determine the difference between the feature representations. Set representation predictor 414 can predict the set representation based in part on the difference between the feature representations. Trainable parameter updater 416 can update the trainable parameters to reduce the difference between the predicted and determined set representations. Window corresponder 420 can correspond windows that may likely share a label, even if that label in unknown, based on the updated trainable parameters. Results presenter 422 can present this information to an external user of the device.

Window corresponder 420 can correspond windows together that likely share a label based on their feature representation analysis. In some embodiments, window corresponder 420 can correspond windows that may share a label even if they come from different measurement sessions or different subjects. Window corresponder 420 can also include other information in its analysis (e.g., window proximity) when corresponding windows. Window corresponder 420 can label data to the extent that it has labeled data with which to work. In some embodiments, window corresponder 420 can attach a probability value to each of the windows indicating the likelihood that said window falls within its alleged grouping.

Results presenter 422 can present information to a user in a variety of means such as through reports, visual display, audio display, or other communicative means. In some embodiments, results presenter 422 presents the information in the form of a Uniform Manifold Approximation and Projection.

System 40 is similar to those of systems 10 and 20 and as such, variations to corresponding components that are described for systems 10 and 20 are equally applicable to system 40.

In accordance with another aspect, there is provided a system for classifying bio-signal data by updating trainable parameters of a neural network. The system has a memory 402 and a computing apparatus 400. Memory 402 is configured to store bio-signal data from one or more subjects. Computing apparatus 400 is configured to receive the bio-signal data from memory 402, define one or more sets of time windows within the bio-signal data using set definer 404, each set including a first anchor window and a sampled window, for at least one set of the one or more sets, determine a determined set representation based in part on the relative position of the first anchor window and the sampled window using set representation determiner 406, extract a feature representation of the first anchor window and a feature representation of the sampled window using an embedder neural network 410 using feature representation extractor 408, aggregate the feature representations using contrastive module 412, and predict a predicted set representation using the aggregated feature representations using set representation predictor 414, update trainable parameters of embedder neural network 410 to minimize a difference between the determined set representation of the at least one set and the predicted set representation of the at least one set using trainable parameter updater 416, correspond at least one time window within the bio-signal data with at least one other time window within the bio-signal data based on the feature representation of the at least one time window and the feature representation of the at least one other time window using the trained embedder neural network 410 using window corresponder 420, and present corresponded time windows using results presenter 422.

In some embodiments, computing apparatus 400 is further configured to store the corresponded time windows in memory 402. In some embodiments, computing apparatus 400 is further configured to transmit the corresponded windows to another computing apparatus.

In some embodiments, the one or more sets of time windows include one or more pairs of time windows, the at least one set of the one or more sets include at least one pair of the one or more pairs, and the computing apparatus determines the determined set representation 6A20 based in part on the relative position of the first anchor window 6A02 and the sampled window 6A12 by defining a positive context region 6A04 and negative context region 6A08 surrounding the first anchor window 6A02, and determining if the sampled window 6A12 is within the positive context region 6A04 or negative context region 6A08.

In some embodiments, the determined set representation is based in part on the relative position of the first anchor window 6A02 and the sampled window 6A12 further includes rejecting the at least one pair if the sampled window 6A12 is not within the positive context region 6A04 or the negative context region 6A08.

In some embodiments, the one or more sets of time windows include one or more triplets of time windows, each triplet further including a second anchor window 6B02b, wherein the second anchor window 6B02b is within a positive context region 6604 surrounding the first anchor window 6B02a, the at least one set of the one or more sets includes at least one triplet of the one or more triplets, the computing apparatus determines the determined set representation 6620 based in part on the relative position of the first anchor window 6B02a and the sampled window 6612 by determining a temporal order of the first anchor window 6B02a, the sampled window 6612, and the second anchor window 6B02b, and the extract the feature representation of the first anchor window 6B02a and a feature representation of the sampled window 6612 using an embedder neural network further includes extracting a feature representation of the second anchor window 6B02b.

In some embodiments, the define the one or more triplets of time windows within the training bio-signal data further includes for one triplet of the one or more triplets, a mirror of the one triplet is included in the one or more triplets.

In some embodiments, the first anchor window includes a series of consecutive anchor windows 6C02, the sampled window includes a series of consecutive sampled windows 6C12, wherein the series of consecutive sampled windows 6C12 is adjacent to the series of consecutive anchor windows 6C02, the set further includes a set of negative sample windows 6C14, and the computing apparatus determines the determined set representation based in part on the relative position of the first anchor window and the sampled window by determining that a given sampled window 6C12a is in the series of sampled windows 6C12. In such embodiments, the extract the feature representation of the first anchor window and a feature representation of the sampled window using an embedder neural network includes extracting a feature representation of each anchor window of the series of consecutive anchor windows 6C02, extracting a feature representation of each sampled window of the series of consecutive sampled windows 6C12, and extracting a feature representation of each negative sample window of the set of negative sample windows 6C14. In such embodiments, the aggregate the feature representations includes embedding the feature representation of each anchor window of the series of anchor windows using an autoregressive embedder 6C24, and aggregating the embedded anchor series, a given feature representation of a given sampled window of the series of sampled windows 6C12, and one or more given feature representations of one or more given negative sample windows 6C14 of the set of negative sample windows. In such embodiments, the predict the predicted set representation includes predicting which of the given feature representations corresponds to the given feature representation of the sampled window of the series of sampled windows 6C12.

In some embodiments, embedder neural network 410 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, computing apparatus 400 includes a server having at least one hardware processor. In some embodiments, computing apparatus 400 can be housed within a server permitting system 40 to update its training.

In some embodiments, classifier 418 includes a classifier neural network, and the update trainable parameters includes updating trainable parameters of the classifier neural network. In such embodiments, system 40 can train embedder neural network 410 and the classifier neural network in an end-to-end manner. In some embodiments, classifying computing apparatus 200 can be configured to receive and implement trained embedder neural network 410 and the classifier neural network.

In some embodiments, contrastive module 412 includes a contrastive neural network, and the update trainable parameters further includes updating trainable parameters of the contrastive neural network. In such embodiments, system 40 can train embedder neural network 410 and the contrastive neural network in an end-to-end manner. In some embodiments, classifying computing apparatus 200 can be configured to receive and implement trained embedder neural network 410 and the contrastive neural network.

Some embodiments further include a display. In such embodiments the present the corresponded time windows includes presenting the corresponded time windows using the display.

In some embodiments, the bio-signal data from one or more subjects further includes associations between personal characteristics of the one or more subjects and the bio-signal data, and the present corresponded time windows includes presenting the personal characteristics associated with the bio-signal data. In some embodiments, the bio-signal data further includes associations between circumstances of measurement (e.g., system operator, location, activity) and the bio-signal data, and the present corresponded time windows includes presenting the circumstances of measurement associated with the bio-signal data.

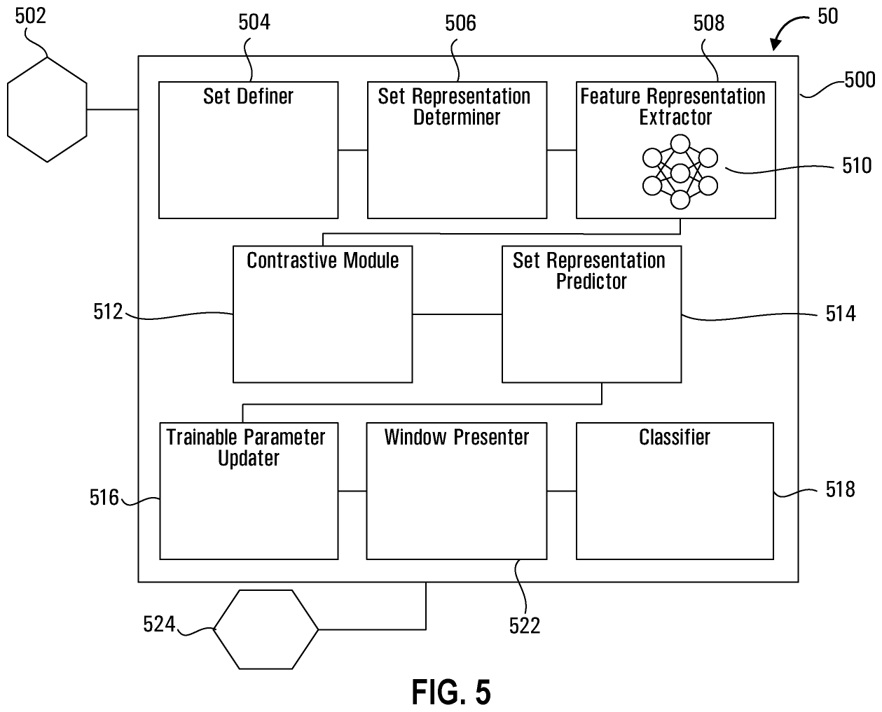

FIG. 5 illustrates a schematic diagram of an apparatus capable of implementing SSL to train a neural network to label bio-signal data using unlabeled data and bio-signal data labeled by a user during operation, according to some embodiments.

System 50 illustrates an exemplary system capable of being configured to group windows in unlabeled bio-signal data, receive labels from a user, and label some or all windows within the bio-signal data based on said received labels. System 50 includes a bio-signal sensor 502, a computing apparatus 500, and an input 524. Computing apparatus 500 is configured to receive bio-signal data from bio-signal sensor 502. Set definer 504 can define sets within the bio-signal data. Set representation determiner 506 can determine the set representations based on the proximity of windows within the set. Feature extractor 508 can extract features from the window using embedder neural network 510. Contrastive module 512 can determine the difference between the feature representations. Set representation predictor 514 can predict the set representation based in part on the difference between the feature representations. Trainable parameter updater 516 can update the trainable parameters to reduce the difference between the predicted and determined set representation. Window presenter 522 presents windows from the bio-signal data to a user. Computing apparatus 500 receives a labeled bio-signal data from the user from input 524. Classifier 518 labels the bio-signal data based on the labeled bio-signal data and the set representations.

In some embodiments, computing apparatus 500 can receive bio-signal data from a memory in addition to, or instead of, from bio-signal sensor 502.

In some embodiments, window presenter 522 can provide information to a user through audio, visual, or other communicative means. One skilled in the art would readily ascertain that the user could be presented with a window via window presenter 522 at any point before classifying using classifier 518 and not necessarily after the trainable parameters have been updated. In some embodiments, window presenter 522 can deduce the minimum number of windows that the user needs to label in order for classifier 518 to be able to label the rest of the bio-signal data and only present those windows to the user. In some embodiments, window presenter 522 will determine a minimum confidence level for labeling the windows and have a user label as many windows as necessary to meet the minimum confidence level while labeling the rest of the bio-signal data. In some embodiments, window presenter 522 can dynamically present windows of high uncertainty to the user.

In some embodiments, input 524 can be a computer keyboard or touch screen. In some embodiments, window presenter 522 can present the user with one window and expect the user to label it using input 524. In some embodiments, window presenter 522 may present the user with one or more windows and expect the user to input which of said windows match a specific label via input 524.

System 50 is similar to those of systems 10, 20, and 40 and as such, variations to corresponding components that are described for systems 10, 20, and 40 are equally applicable to system 50.

In accordance with another aspect, there is provided an apparatus for classifying bio-signal data by updating trainable parameters of a neural network. The apparatus has a bio-signal sensor 502 and a computing apparatus 500. Bio-signal sensor 502 is configured to receive bio-signal data from a subject. The bio-signal data includes unlabeled bio-signal data. Computing apparatus 500 is configured to receive the bio-signal data from the subject, define one or more sets of time windows within the bio-signal data using set definer 504, each set including a first anchor window and a sampled window, for at least one set of the one or more sets, determine a determined set representation based in part on the relative position of the first anchor window and the sampled window using set representation determiner 506, extract a feature representation of the first anchor window and a feature representation of the sampled window using an embedder neural network 510 using feature representation extractor 508, aggregate the feature representations using contrastive module 512, and predict a predicted set representation using the aggregated feature representations using set representation predictor 514, update trainable parameters of the embedder neural network 510 to minimize a difference between the determined set representation of the at least one set and the predicted set representation of the at least one set using trainable parameter updater 516, present the bio-signal data to a user using window presenter 522, receive at least one label from the user via input 524 to generate one or more labeled windows within the bio-signal data, and label the unlabeled bio-signal data using classifier 518, the one or more labeled windows, and embedder neural network 510. The set representation denotes likely label correspondence between the first anchor window and the sampled window.

In some embodiments, computing apparatus 500 is further configured to store the bio-signal data labelled using classifier 518 in a memory. In some embodiments, computing apparatus 500 is further configured to transmit the bio-signal data labelled using classifier 518 to another computing apparatus.

In some embodiments, the one or more sets of time windows include one or more pairs of time windows, the at least one set of the one or more sets include at least one pair of the one or more pairs, and the computing apparatus determines the determined set representation 6A20 based in part on the relative position of the first anchor window 6A02 and the sampled window 6A12 by defining a positive context region 6A04 and negative context region 6A08 surrounding the first anchor window 6A02, and determining if the sampled window 6A12 is within the positive context region 6A04 or negative context region 6A08.

In some embodiments, the determined set representation is based in part on the relative position of the first anchor window 6A02 and the sampled window 6A12 further includes rejecting the at least one pair if the sampled window 6A12 is not within the positive context region 6A04 or the negative context region 6A08.

In some embodiments, the one or more sets of time windows include one or more triplets of time windows, each triplet further including a second anchor window 6B02b, wherein the second anchor window 6B02b is within a positive context region 6604 surrounding the first anchor window 6B02a, the at least one set of the one or more sets includes at least one triplet of the one or more triplets, the computing apparatus determines the determined set representation 6620 based in part on the relative position of the first anchor window 6B02a and the sampled window 6612 by determining a temporal order of the first anchor window 6B02a, the sampled window 6612, and the second anchor window 6B02b, and the extract the feature representation of the first anchor window 6B02a and a feature representation of the sampled window 6612 using an embedder neural network further includes extracting a feature representation of the second anchor window 6B02b.

In some embodiments, define the one or more triplets of time windows within the training bio-signal data further includes for one triplet of the one or more triplets, a mirror of the one triplet is included in the one or more triplets.

In some embodiments, the first anchor window includes a series of consecutive anchor windows 6C02, the sampled window includes a series of consecutive sampled windows 6C12, wherein the series of consecutive sampled windows 6C12 is adjacent to the series of consecutive anchor windows 6C02, the set further includes a set of negative sample windows 6C14, and the computing apparatus determines the determined set representation based in part on the relative position of the first anchor window and the sampled window by determining that a given sampled window 6C12a is in the series of sampled windows 6C12. In such embodiments, the extract the feature representation of the first anchor window and a feature representation of the sampled window using an embedder neural network includes extracting a feature representation of each anchor window of the series of consecutive anchor windows 6C02, extracting a feature representation of each sampled window of the series of consecutive sampled windows 6C12, and extracting a feature representation of each negative sample window of the set of negative sample windows 6C14. In such embodiments, the aggregate the feature representations includes embedding the feature representation of each anchor window of the series of anchor windows using an autoregressive embedder 6C24, and aggregating the embedded anchor series, a given feature representation of a given sampled window of the series of sampled windows 6C12, and one or more given feature representations of one or more given negative sample windows 6C14 of the set of negative sample windows. In such embodiments, the predict the predicted set representation includes predicting which of the given feature representations corresponds to the given feature representation of the sampled window of the series of sampled windows 6C12.

In some embodiments, embedder neural network 510 can be one of a convolutional neural network, a fully connected neural network, and a recurrent neural network.

In some embodiments, the bio-signal data includes at least one of EEG, EKG, breath, heartrate, PPG, skin conductance, EOG, chin EMG, respiration airflow, and oxygen saturation data. In some embodiments the bio-signal data can include multiple types of bio-signal data. In some embodiments, the bio-signal data can include a brain state of the subject during observations (e.g., where data has been preprocessed first by other already existing classifiers whose predictions could be fed into the system alongside the bio-signal data).

In some embodiments, the label the user bio-signal data includes determining a brain state of the user. In some embodiments, the label the user bio-signal data includes determining a sleep state of the user. In some embodiments, the label the user bio-signal data includes detecting a pathology of the user. In such embodiments the prediction of the brain state, sleep state, or pathology of the user could be a downstream task that is performed once the system has labelled the bio-signal data. The labels provided by the system can be fed as input into another system in order to make brain state, sleep state, pathology, or other predictions about the user.

In some embodiments, training computing apparatus 500 includes a server having at least one hardware processor. In some embodiments, training computing apparatus 500 can be housed within a server permitting system 50 to update its training. In some embodiments, said server can push or otherwise make accessible the latest trained system for one or more classifying computing apparatuses 200.

In some embodiments, training computing apparatus 500 includes a server configured to upload embedder neural network 510 and classifier 518 to the classifier computing apparatus 200.

In some embodiments, training computing apparatus 500 includes classifying computing apparatus 200.

In some embodiments, classifier 518 includes a classifier neural network, and the update trainable parameters includes updating trainable parameters of the classifier neural network. In such embodiments, system 50 can train embedder neural network 510 and the classifier neural network in an end-to-end manner. In some embodiments, classifying computing apparatus 200 can be configured to receive and implement trained embedder neural network 510 and the classifier neural network.

In some embodiments, contrastive module 512 includes a contrastive neural network, and the update trainable parameters further includes updating trainable parameters of the contrastive neural network. In such embodiments, system 50 can train embedder neural network 510 and the contrastive neural network in an end-to-end manner. In some embodiments, classifying computing apparatus 200 can be configured to receive and implement trained embedder neural network 510 and the contrastive neural network.

In some embodiments, classifier 518 labels unlabeled bio-signal data based in part on a personal characteristic of the subject corresponding to the training bio-signal data, and the classifying the user bio-signal data is based in part on a personal characteristic of the user. In some embodiments, other circumstances of measurement (e.g., system operator, location, activity) can be used to label unlabeled bio-signal data. In some embodiments, characteristics (personal or circumstances of measurement) can be used by the system to label bio-signal data.

In some embodiments, classifier 518 includes a classifying neural network, and computing apparatus 500 is further configured to update trainable parameters of the classifying neural network to minimize a difference between labels of the one or more labeled windows in the bio-signal data and predicted labels of the one or more labeled windows in the bio-signal data. In such embodiments, system 50 can train embedder neural network 510 and the classifier neural network in an end-to-end manner.

The following describes non-limiting, exemplary research.

### Methods

### Example Self-Supervised Learning Approaches

Although it has not always been known as such, SSL is used in many fields. In computer vision, multiple approaches have been proposed that rely on the spatial structure of images and the temporal structure of videos. For example, a context prediction task can be used to train feature extractors on unlabeled images by predicting the position of a randomly sampled image patch relative to a second patch. Using this approach to pretrain a neural network, improved performance as compared to a purely supervised model on the Pascal VOC object detection challenge can be achieved. These results were among the first showing that self-supervised pretraining could help improve performance when limited annotated data is available. Similarly, the jigsaw puzzle task led to improved downstream performance on the same dataset. In the realm of video processing, approaches based on temporal structure can be used: for instance, predicting whether a sequence of video frames were ordered or shuffled can be used as a pretext task and tested on a human activity recognition downstream task.

Similarly, modern natural language processing (NLP) tasks often rely on self-supervision to learn word embeddings, which are at the core of many applications. For instance, the original word2vec model can be trained to predict the words around a center word or a center word based on the words around it, and then reused on a variety of downstream tasks. A dual-task self-supervised approach, BERT, can lead to state-of-the-art performance on 11 NLP tasks such as question answering and named entity recognition. The high performance achieved by this approach showcases the potential of SSL for learning general-purpose representations.

More general pretext tasks as well as improved methodology can lead to strong results that have begun to rival purely supervised approaches. For instance, contrastive predictive coding (CPC), an autoregressive prediction task in latent space, can be successfully used for images, text and speech. Given an encoder and an autoregressive model, the task consists of predicting the output of the encoder for future windows (or image patches or words) given a context of multiple windows. Such approaches can provide several improved results on various downstream tasks and further show that higher-capacity networks could improve downstream performance even more, especially in low-labeled data regimes. Momentum contrast (MoCo), rather than proposing a new pretext task, is an improvement upon contrastive tasks, i.e., where a classifier must predict which of two or more inputs is the true sample. By improving the sampling of negative examples in contrastive tasks, MoCo can help boost the efficiency of SSL training as well as the quality of the representations learned. Similarly, using the right data augmentation transforms (e.g., random cropping and color distortion on images) and increasing batch size can lead to significant improvements in downstream performance.

The ability of SSL-trained features to demonstrably generalize to downstream tasks justifies a closer look at their statistical structure. A general and theoretically grounded approach from the perspective of nonlinear independent components analysis is as follows. An observation x is embedded using an invertible neural network, and contrasted against an auxiliary variable u (e.g., the time index, the index of a segment or the history of the data). A discriminator classifies the pair by learning to predict whether x is paired with its corresponding auxiliary variable u or a perturbed (random) one u*. When the data exhibits certain structure (e.g., autocorrelation, non-stationarity, non-gaussianity), the embedder trained on this contrastive task will perform identifiable nonlinear ICA. Most of the previously introduced SSL tasks can be viewed through this framework. Given the widespread use of linear ICA as a preprocessing and feature extraction tool in the EEG community, an extension to the nonlinear regime is a step forward and could help improve traditional processing pipelines.

A model inspired by word2vec, called wave2vec, was developed to work with EEG and electrocardiography (ECG) time series. Representations are learned by predicting the features of neighbouring windows from the concatenation of time-frequency representations of EEG signals and demographic information. This approach is however only implemented with a single EEG dataset and was not benchmarked against fully supervised deep learning approaches or expert feature classification. SSL can be applied to ECG as a way to learn features for a downstream emotion recognition task: a transformation discrimination pretext task is used in which the model had to predict which transformations had been applied to the raw signal.

Over other approaches include that the method presented herein is simpler and more efficient to use, both at training and testing time. Other approaches require a feature extraction step, autoencoder pretraining, and demographic information associated with the data. The method presensed herein makes use of both proximity and “farness” to learn representations which is intuitively more adapted to processing bio-signal time series.

Self-Supervised Learning Pretext Tasks for EEG

The three SSL pretext tasks used herein will now be described. A visual explanation of the tasks can be found in FIG. 6A, FIG. 6B, and FIG. 6C.

FIG. 6 is a visual explanation of three SSL pretext tasks used herein with FIG. 6A showing Relative Positioning, FIG. 6B showing Temporal Shuffling, and FIG. 6C showing Contrastive Predictive Coding (CPC). The first column of each figure illustrates the sampling process by which examples are obtained in each pretext task. The second column describes the training process, where sampled examples are used to train a feature extractor h_{Θ} end-to-end.

Notation

Notation herein denotes by [symbol] q [symbol] the set {1, . . . , q} and by p, q the set {p, . . . , q} for any integer p, q∈ [symbol] . The index t refers to time indices in the multivariate time series S∈ [symbol] ^{C×M}, where M is the number of time samples and C is the dimension of samples (channels). For simplicity, it is assumed that each S has the same size. y∈{−1, 1} denotes a binary label used in the learning task.

Relative Positioning

FIG. 6A is a visual explanation of Relative Positioning. The first column illustrates the sampling process by which examples are obtained in each pretext task. The second column describes the training process, where sampled examples are used to train a feature extractor h_{Θ} end-to-end. Pairs of windows are sampled from S such that the two windows of a pair are either close in time (‘positive pairs’) or farther away (‘negative pairs’). h_{Θ} is then trained to predict whether a pair is positive or negative.

To produce labeled samples from the multivariate time series S, pairs of time windows (xₜ, x_{t′}) are sampled where each window xₜ, x_{t′} is in [symbol] ^{C×T}, and T is the duration of each window, and where the index t indicates the time sample at which the window starts in S. The first window xₜ is referred to as the “anchor window”. An assumption is that an appropriate representation of the data should evolve slowly over time (akin to the driving hypothesis behind Slow Feature Analysis (SFA)) suggesting that time windows close in time should share the same label. In the context of sleep staging, for instance, sleep stages usually last between 1 to 40 minutes; therefore, nearby windows likely come from the same sleep stage, whereas faraway windows likely come from different sleep stages. Given τₚₒₛ∈ [symbol] , which controls the duration of the positive context, and τ_{neg}∈ [symbol] , which corresponds to the negative context around each window xᵢ, N labeled pairs are sampled:

[symbol]

_{N}={((xₜ_{ᵢ},xₜ_{ᵢ}_{′})|i∈ [symbol] N [symbol] ,(tᵢ,t′ᵢ)∈ [symbol] ,yᵢ∈ [symbol] },

where [symbol] ={−1,1} and [symbol] ={(t, t′)∈ [symbol] M−T+1 [symbol] ²∥t−t′|≤τₚₒₛ or |t−t′|>τ_{neg}}. [symbol] is the set of all pairs of time indices (t, t′) which can be constructed from windows of size T in a time series of size M, given the duration constraints imposed by the particular choices of τₚₒₛ and τ_{neg}. The values of τₚₒₛ and τ_{neg} can be selected based on prior knowledge of the signals and/or with a hyperparameter search. Here yᵢ∈ [symbol] is specified by the positive or negative contexts parameters:

$${y}_{i} = \{ \begin{matrix}1. & if \vert {t}_{i} - {t}_{i}^{′} \vert \leq {τ}_{pos} \\ - 1. & if \vert {t}_{i} - {t}_{i}^{′} \vert > {τ}_{neg}\end{matrix} \qquad ( 1 )$$

Window pairs may be ignored where xₜ, falls outside of the positive and negative contexts of the anchor window xₜ. In other words, the label indicates whether two time windows are closer together than τₚₒₛ or farther apart than τ_{neg} in time. This pretext task can be referred to as “relative positioning” (RP).

In order to learn end-to-end how to discriminate pairs of time windows based on their relative position, two functions h_{Θ} and g_{RP} are introduced. h_{Θ}: [symbol] ^{C×T}→ [symbol] _{D} is a feature extractor with parameters Θ which maps a window x to its representation in the feature space. Ultimately, it is expected for h_{Θ} to learn an informative representation of raw EEG input which can be reused in different downstream tasks. A contrastive module g_{RP} is then used to aggregate the feature representations of each window. For the RP task, g_{RP}: [symbol] ^{D}× [symbol] ^{D}→ [symbol] ^{D} combines representations from pairs of windows by computing an elementwise absolute difference, denoted by the |·| operator: g_{RP}(h_{Θ}(x), h_{Θ}(x′))=|h_{Θ}(x)−h_{Θ}(x′)|∈ [symbol] ^{D}. The role of g_{RP} is to aggregate the feature vectors extracted by h_{Θ} on the two input windows and highlight their differences to simplify the contrastive task. Finally, a linear context discriminative model with coefficients w∈ [symbol] ^{D} and bias term w₀∈ [symbol] is responsible for predicting the associated target y. Using the binary logistic loss on the predictions of g_{RP} a joint loss function [symbol] (Θ, w, w₀) can be written as

[symbol]

(Θ,w,w₀)= [symbol] log(1+exp(−y[w^{T}g_{RP}(h_{Θ}(xₜ),h_{Θ}(x_{t′}))+w₀])) (2)

which is assumed to be fully differentiable with respect to the parameters (Θ, w, w₀). Given the convention used for y, the predicted target is the sign of w^{T}g(h_{Θ}(xₜ),h_{Θ}(x_{t′}))+w₀.

Temporal Shuffling

FIG. 6B is a visual explanation of Temporal Shuffling. The first column illustrates the sampling process by which examples are obtained in each pretext task. The second column describes the training process, where sampled examples are used to train a feature extractor h_{Θ} end-to-end. Triplets of windows (rather than pairs) are sampled from S. A triplet is given a positive label if its windows are ordered or a negative label if they are shuffled. h_{Θ} is then trained to predict whether the windows of a triplet are ordered or shuffled.

In some embodiments, there is provided a variation of the RP task referred to as “temporal shuffling” (TS), in which two anchor windows xₜ and x_{t″} from the positive context, and a third window xₜ, that is either between the first two windows or in the negative context are sampled. Window triplets are constructed that are either temporally ordered (t<t′<t″) or shuffled (t<t″<t′ or t′<t<t″). The number of possible triplets can be augmented by also considering the mirror image of the previous triplets, e.g., (xₜ, x_{t′}, x_{t″}) becomes (x_{t″},x_{t′},xₜ). The label yᵢ then indicates whether the three windows are ordered or have been shuffled.

The contrastive module for TS is defined as g_{TS}: [symbol] ^{D}× [symbol] _{D}× [symbol] ^{D}→ [symbol] ^{2D} and is implemented by concatenating the absolute differences:

g_{TS}(h_{Θ}(x),h_{Θ}(x′),h_{Θ}(x″))=(|h_{Θ}(x)−h_{Θ}(x′)|,|h_{Θ}(x′)−h_{Θ}(x″)|)∈ [symbol] ^{2D}.

Moreover, Eq. (2) is extended to TS by replacing g_{RP} by g_{TS} and introducing x_{t″} to obtain:

[symbol]

(Θ,w,w₀)= [symbol] log(1+exp(−y[w^{T}g_{TS}(h_{Θ}(xₜ),h_{Θ}(x_{t′}),hΘ(x_{t″}))+w₀])), (3)

Contrastive Predictive Coding

FIG. 6C is a visual explanation of Contrastive Predictive Coding. The first column illustrates the sampling process by which examples are obtained in each pretext task. The second column describes the training process, where sampled examples are used to train a feature extractor h_{Θ} end-to-end. Sequences of N_{c}+Nₚ consecutive windows are sampled from S along with random distractor windows (‘negative samples’). Given the first N_{c} windows of a sequence (the ‘context’), a neural network is trained to identify which window out of a set of distractor windows actually follows the context.

The contrastive predictive coding (CPC) pretext task, is defined here in comparison to RP and TS, as all three tasks share key similarities. Indeed, CPC can be seen as an extension of RP, where the single anchor window xₜ is replaced by a sequence of N_{c} non-overlapping windows that are summarized by an autoregressive encoder g_{AR}: [symbol] ^{D×N}^{_{C}}→ [symbol] ^{D}^{_{AR}} with parameters Θₐᵣ. CPC's encoder g_{AR} has parameters Θ_{AR}, however they are omitted from the notation for brevity. This way, the information in the context can be represented by a single vector cₜ∈ [symbol] ^{D}^{_{AR}}. g_{AR} can be implemented for example as a recurrent neural network with gated-recurrent units (GRU).

The context vector cₜ is paired with not one, but Nₚ future windows (or “steps”) which immediately follow the context. Negative windows are then sampled in a similar way as with RP and TS when τ_{neg}=0, i.e., the negative context is relaxed to include the entire timeseries. For each future window, N_{b} negative windows x* are sampled inside each multivariate time series S (“same-recording negative sampling”) or across all available S (“across-recording negative sampling”).

For the sake of simplicity and to follow the notation of the original CPC article, notation is modified slightly: a time window is now denoted by xₜ where t is the index of the window in the list of all non-overlapping windows of size T that can be extracted from a time series S. Therefore, the procedure for building a dataset with N examples boils down to sampling sequences X^{c}, X^{p} and Xⁿ in the following manner:

Xᵢ^{c}=(xₜ_{ᵢ}_{-N}_{_{c}}₊₁, . . . ,xₜ_{ᵢ}) (N_{c} context windows))

Xᵢ^{p}=(xₜ_{ᵢ}₊₁, . . . ,xₜ_{ᵢ}_{+N}_{ₚ}) (Nₚ future windows)

$${X}_{i}^{n} = ( {x}_{{t}_{{i}_{1 , 1}}^{*}} , … , {x}_{{t}_{{i}_{1 , {N}_{b}}}^{*}} , … , {x}_{{t}_{{i}_{{N}_{p , 1}}}^{*}} , … , {x}_{{t}_{{i}_{{N}_{p} , {N}_{b}}}^{*}} )$$

(NₚN_{b} random negative windows)
where tᵢ∈N_{c}, M−Nₚ. Time indices of windows sampled uniformly at random are denoted with t*. The dataset then reads:

Z_{N}={(Xᵢ^{c},Xᵢ^{p},Xᵢⁿ)|i∈ [symbol] N [symbol] }. (4)

As with RP and TS, the feature extractor h_{Θ} is used to extract a representation of size D from a window xₜ. Finally, whereas the contrastive modules g_{RP} and g_{TS} explicitly relied on the absolute value of the difference between embeddings h, here for each future window xₜ₊ₖ where k∈ [symbol] Nₚ [symbol] a bilinear model fₖ parametrized by Wₖ∈^{D×D}^{_{AR}} is used to predict whether the window chronologically follows the context cₜ or not:

fₖ(cₜ,h_{Θ}(xₜ₊ₖ))=h_{Θ}(xₜ₊ₖ)^{T}Wₖcₜ (5)

The whole CPC model can be trained end-to-end using the InfoNCE loss (a categorical cross-entropy loss) defined as

$$ℒ ( ⊖ , {⊖}_{NR} , {W}_{k} , … , {W}_{k \cdot {N}_{p}} \text{?} ) = - \text{?} log [ \text{?} ] \\ \text{?} \text{indicates text missing or illegible when filed}$$

While in RP and TS the model can predict whether a pair is positive or negative, in CPC the model can pick which of N_{b}+1 windows actually follows the context. In practice, batches of N_{b}+1 sequences are sampled and for each sequence the N_{b} other sequences in the batch are used to supply negative examples.

Downstream Tasks

Empirical benchmarks of EEG-based SSL were performed on two clinical problems that are representative of the current challenges in machine learning-based analysis of EEG: sleep monitoring and pathology screening. These two clinical problems commonly give rise to classification tasks, albeit with different numbers of classes and distinct data-generating mechanisms: sleep monitoring is concerned with biological events (event level) while pathology screening is concerned with single patients as compared to the population (subject level). These two clinical problems have generated considerable attention in the research community, which has led to the curation of large public databases. To enable fair comparison with supervised approaches, SSL was benchmarked on the Physionet Challenge 2018 and the TUH Abnormal EEG datasets.

First, sleep staging was considered, which is a critical component of a typical sleep monitoring assessment and is key to diagnosing and studying sleep disorders such as apnea and narcolepsy. Sleep staging has been extensively studied in the machine (and deep) learning literature, though not through the lens of SSL. Achieving fully automated sleep staging could have a substantial impact on clinical practice as (1) agreement between human raters is often limited and (2) the annotation process is time-consuming and still largely manual. Sleep staging typically gives rise to a 5-class classification problem where the possible predictions are W (wake), N1, N2, N3 (different levels of sleep) and R (rapid eye movement periods). Here, the task consists of predicting the sleep stages that correspond to 30-s windows of EEG.

Second, SSL was applied to pathology detection: EEG is routinely used in a clinical context to screen individuals for neurological conditions such as epilepsy and dementia. However, successful pathology detection requires highly specialized medical expertise and its quality depends on the expert's training and experience. Automated pathology detection could, therefore, have a major impact on clinical practice by facilitating neurological screening. This gives rise to classification tasks at the subject level where the challenge is to infer the patient's diagnosis or health-status from the EEG recording. In the TUH dataset, medical specialists have labeled recordings as either pathological or non-pathological, giving rise to a binary classification problem. Importantly, these two labels reflect highly heterogeneous situations: a pathological recording could reflect anomalies due to various medical conditions, suggesting a rather complex data-generating mechanism. Various supervised approaches, some of them leveraging deep architectures, have addressed this task, although none has relied on self-supervision.

These two tasks are further described in the section on “Data” used in experiments, discussed below.

Deep Learning Architectures

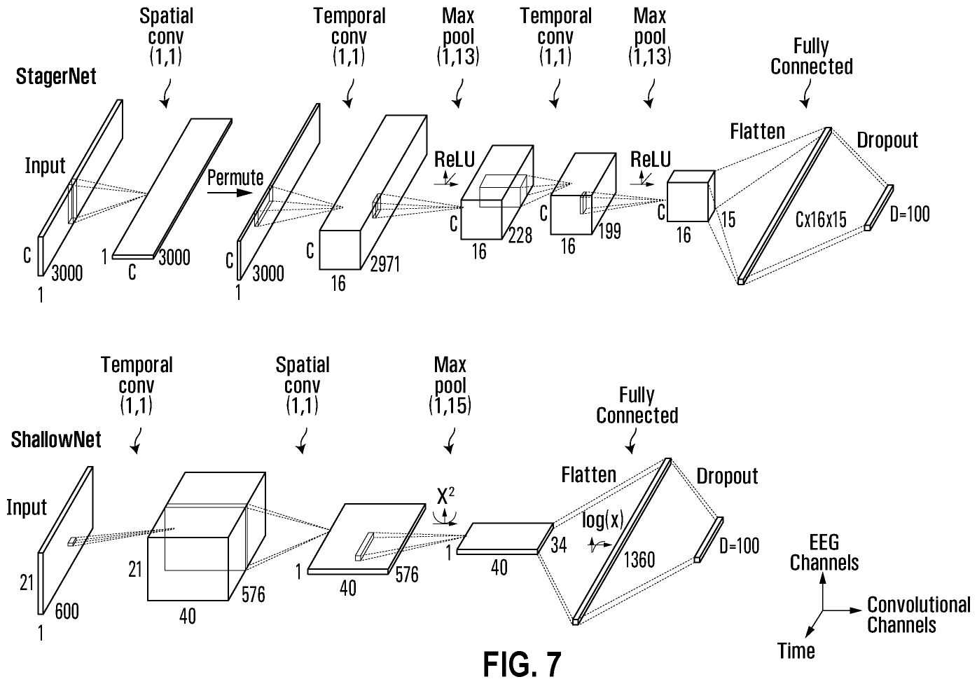

Two different deep learning architectures were used as embedders h_{Θ} in experiments (see FIG. 7). Both architectures are convolutional neural networks composed of spatial and temporal convolution layers, which respectively learn to perform the spatial and temporal filtering operations typical of EEG processing pipelines.

FIG. 7 illustrates neural network architectures used as embedder h_{θ} for (1) sleep EEG and (2) pathology detection experiments.

The first one, called StagerNet, was adapted from previous work on sleep staging where it was shown to perform well for window-wise classification of sleep stages. StagerNet is a 3-layer convolutional neural network optimized to process windows of 30-s of multichannel EEG. As opposed to the original architecture, (1) twice as many convolutional channels (16 instead of 8) were used, (2) batch normalization is added after both temporal convolution layers (3) temporal convolutions were not padded and (4) the dimensionality of the output layer was changed to D=100 instead of the number of classes (see FIG. 7, item (1)). This yielded a total of 62,307 trainable parameters. More specifically, a spatial convolution layer with as many filters as input channels extracted spatial information from the input time windows. Two blocks to enable filtering along the time dimension were then applied, each one consisting of a temporal convolution layer followed by max-pooling and a rectified linear unit non-linearity. The output was finally flattened and passed to a fully connected layer with dropout to produce the output feature vector.

Batch normalization can harm the network's ability to learn on the CPC pretext task. However, this effect was not observed on models described herein (likely because their capacity is relatively small) and alternatives such as no normalization or layer normalization performed defavorably. Therefore, batch normalization was also used in CPC experiments.

The second embedder architecture is ShallowNet, previously used with TUH Abnormal dataset. Originally designed to be a parametrized version of the filter bank common spatial patterns (FBCSP) processing pipeline common in brain-computer interfacing, ShallowNet has a single (split) convolutional layer followed by a squaring non-linearity, average pooling, a logarithm non-linearity, and a linear output layer. Batch normalization was used after the temporal convolution layer. Despite its simplicity, this architecture was shown to perform almost as well as the best model on the task of pathology detection on the TUH Abnormal dataset. Therefore it was used as is, except for the dimensionality of the output layer which was changed to D=100 (see FIG. 7, item (2)). This yielded a total of 170,860 trainable parameters.

A GRU was used with a hidden layer of size D_{AR}=100 for the CPC task's g_{AR}, for experiments on both datasets.

The Adam optimizer with β₁=0.9 and β₂=0.999 and learning rate 5×10⁻⁴ was used. The batch size for all deep models was set to 256, except for CPC where it was set to 32. Training ran for at most 150 epochs, or until the validation loss stopped decreasing for a period of a least 10 epochs (or 6 epochs for CPC). Dropout was applied to fully connected layers at a rate of 50% and a weight decay of 0.001 was applied to the trainable parameters of all layers. Finally, the parameters of all neural networks were randomly initialized using uniform He initialization.

Baselines

The SSL tasks were compared to four baseline approaches on the downstream tasks: (1) random weights, (2) convolutional autoencoders, (3) purely supervised learning and (4) handcrafted features.

The random weights baseline used an embedder whose weights were frozen after random initialization. The autoencoder (AE) was a more basic approach to representation learning, where a neural network made up of an encoder and a decoder learned an identity mapping between its input and its output, penalized by e.g., a mean squared error loss. Here, h_{Θ} was used as the encoder and a convolutional decoder designed that inverts the operations of h_{Θ}. The purely supervised model was directly trained on the downstream classification problem, i.e., it has access to the labeled data. To do so, an additional linear classification layer was added to the embedder, before training the whole model with a multi-class cross entropy loss.

Finally, traditional machine learning baselines were also included based on handcrafted features. For sleep staging, the following features were extracted: mean, variance, skewness, kurtosis, standard deviation, frequency log-power bands between (0.5, 4.5, 8.5, 11.5, 15.5, 30) Hz as well as all their possible ratios, peak-to-peak amplitude, Hurst exponent, approximate entropy and Hjorth complexity. This resulted in 37 features per EEG channel, which were concatenated into a single vector. In the event of an artefact causing missing values in the feature vector of a window, missing values were imputed feature-wise using the mean of the feature computed over the training set. For pathology detection, Riemannian geometry features were used where a non-linear classifier trained on tangent space features reached high accuracy on the evaluation set of the TUH Abnormal dataset. The covariance matrices per recording were not averaged to allow a fair comparison with the other methods which work window-wise. Therefore, for C channels of EEG, the input to the classifier had dimensionality C(C+1)/2.

For the downstream tasks, features learned with RP, TS, CPC and AE were classified using linear logistic regression with L2-regularization parameter C=1, while handcrafted features were classified using a random forest classifier with 300 trees, maximum depth of 15 and a maximum number of features per split of \sqrt{F} (where F is the number of features). Varying C had little impact on downstream performance, and therefore a value of 1 was used across experiments. Random forest hyperparameters were selected using a grid search with maximum depth in {3,5,7,9,11,13,15}, and maximum number of features per tree in {\sqrt{F}, log₂F} using the validation sets as described in the “Data” section below. Balanced accuracy (bal acc), defined as the average per-class recall, was used to evaluate model performance on the downstream tasks. Moreover, during training, the loss was weighted to account for class imbalance. Models were trained using a combination of the braindecode, MNE-Python, pytorch, pyRiemann and scikit-learn packages. Finally, deep learning models were trained on 1 or 2 Nvidia Tesla V100 GPUs for anywhere from a few minutes to 7 h, depending on the amount of data, early stopping and GPU configuration.

Semi-Supervised Baseline in Data Quantity Experiments

SSL-learned features were used in a semi-supervised setting, i.e. where a limited amount of labeled data is used in conjunction with a larger set of unlabeled examples. An additional baseline is included that draws on self-training, a well-established semi-supervised approach (label propagation was also attempted, however this approach did not scale well to the dimensionality of the problem and was consequently dropped). In self-training, a classifier trained with a limited number of labeled examples is used to predict labels on the unlabeled examples. Examples for which the classifier produces predictions with a high enough confidence are then added to the training set, and the model is trained again. This process can be repeated up to a specific number of times, or until no new example is added during the prediction phase.

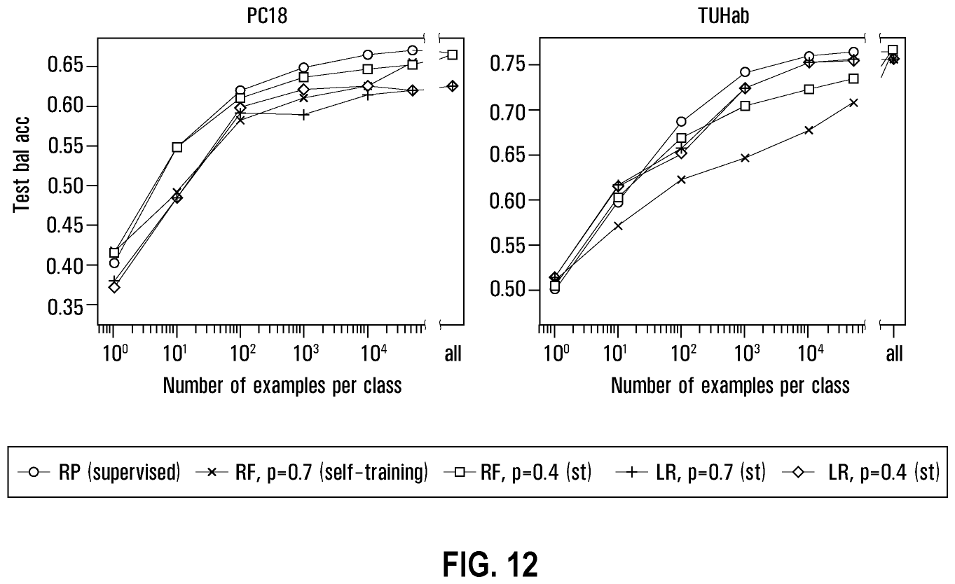

FIG. 12 illustrates the impact of number of labeled examples per class on downstream performance for a self-training semi-supervised baseline, as compared to the handcrafted features approach. Self-training experiments were conducted with a random forest (RF) and logistic regression (LR) using the same hyperparameters as described in the section titled “Baselines” and a probability threshold of 0.7 or 0.4 and maximum number of iterations of 5. Self-training overall harmed downstream performance for both datasets.

Self-training is compared to the handcrafted baseline in FIG. 12. Instead of improving performance, self-training systematically led to a decrease in performance as compared to the handcrafted baseline approach, except for minimal improvements when only one or 10 labeled examples were available per class. Increasing the threshold mitigated this decrease, but did not lead to improved performance over a purely supervised model either. Given a potential limitation arising from the probability estimation accuracy of tree-based models, a logistic regression classifier was also tested which should produce more reliable probability estimates. Performance was overall negatively impacted again.

These experiments suggest that while semi-supervised approaches have shown potential to leverage large amounts of unlabeled data, their use is not straightforward in EEG classification problems and domain-specific efforts might have to be made in order to accommodate their use.

Data

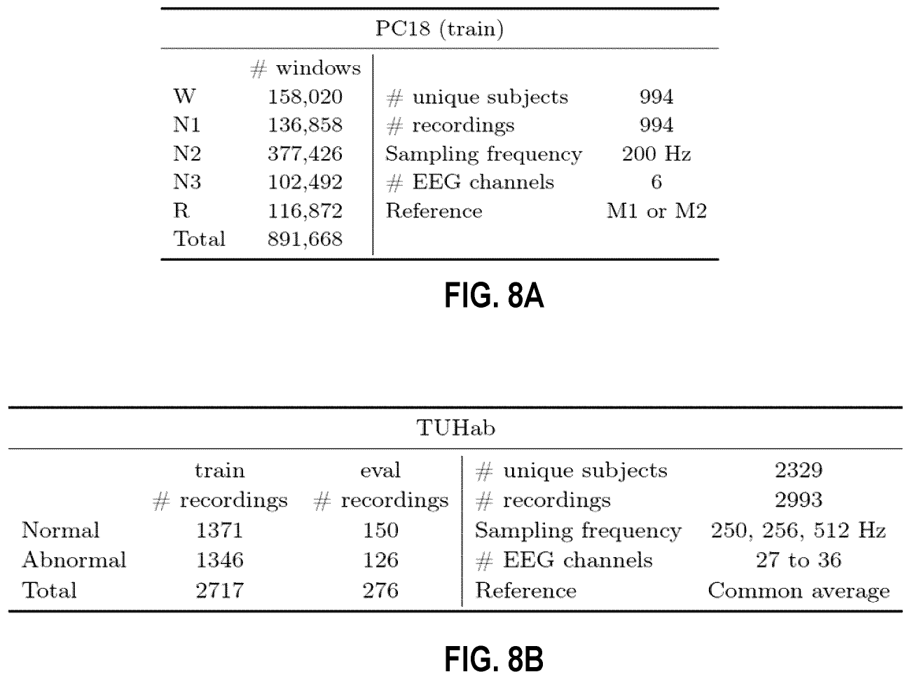

Experiments conducted on two publicly available EEG datasets are described in the tables illustrated in FIG. 8A and FIG. 8B.

Physionet Challenge 2018 Dataset

FIG. 8A is a table describing the Physionet Challenge 2018 (PC18) dataset used in this study for sleep staging experiments.

Sleep staging experiments were conducted on the Physionet Challenge 2018 (PC18) dataset. This dataset was initially released in the context of an open-source competition on the detection of arousals in sleep recordings, i.e., short moments of wakefulness during the night. A total of 1,983 different individuals with (suspected) sleep apnea were monitored overnight and their EEG, EOG, chin EMG, respiration airflow and oxygen saturation measured. Specifically, 6 EEG channels from the international 10/20 system were recorded at 200 Hz: F3-M2, F4-M1, C3-M2, C4-M1, O1-M2 and O2-M1. The recorded data was then annotated by 7 trained scorers following the AASM manual into sleep stages (W, N1, N2, N3 and R). Moreover, 9 different types of arousal and 4 types of sleep apnea events were identified in the recordings. As the sleep stage annotations are only publicly available on about half the recordings (used as the training set during the competition), analysis was focused on these 994 recordings. In this subset of the data, mean age is 55 years old (min: 18, max: 93) and 33% of participants are female.

TUH Abnormal EEG Dataset

FIG. 8B is a table describing the TUH Abnormal (TUHab) dataset used in EEG pathology detection experiments.

The TUH Abnormal EEG dataset v2.0.0 (TUHab) was used to conduct experiments on pathological EEG detection. This dataset contains 2,993 recordings of 15 minutes or more from 2,329 different patients who underwent a clinical EEG in a hospital setting. Each recording was labeled as “normal” (1,385 recordings) or “abnormal” (998 recordings) based on detailed physician reports. Most recordings were sampled at 250 Hz (although some were sampled at 256 or 512 Hz) and contained between 27 and 36 electrodes. Moreover, the corpus is divided into a training and an evaluation set with 2,130 and 253 recordings each. The mean age across all recordings is 49.3 years old (min: 1, max: 96) and 53.5% of recordings are of female patients.

Data Splits and Sampling

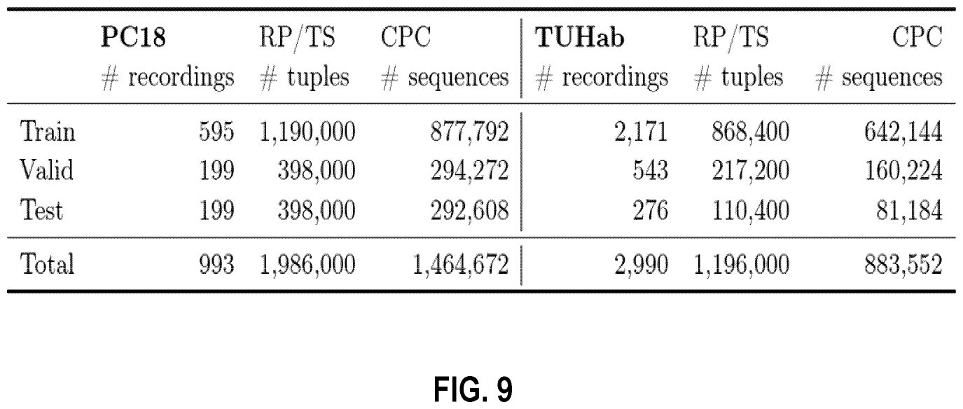

The available recordings from PC18 and TUHab were split into training, validation and testing sets such that the examples from each recording are only in one of the sets (as shown in the Table of FIG. 9).

For PC18, a 60-20-20% random split was used, meaning there were 595, 199 and 199 recordings in the training, validation and testing sets respectively. For RP and TS, 2000 pairs or triplets of windows were sampled from each recording. For CPC, the number of batches to extract from each recording was computed as 0.05 times the number of windows in that recording; moreover, the batch size was set to 32.

For TUHab, the provided evaluation set was used as a test set. The recordings of the development set were split 80-20% into a training and a validation set. Therefore, 2,171, 543 and 276 recordings were used in the training, validation and testing sets. Since the recordings are shorter for TUHab, 400 RP pairs or TS triplets were randomly sampled instead of 2000 from each recording. The same CPC sampling parameters were used as for PC18.

Preprocessing

The preprocessing of the EEG recordings differed for the two datasets. On PC18, the raw EEG was first filtered using a 30 Hz FIR lowpass filter with a Hamming window, to reject higher frequencies that are not critical for sleep staging. The EEG channels were then downsampled to 100 Hz to reduce the dimensionality of the input data. For the same reason, analysis was focused on channels F3-M2 and F4-M1, as being closer to the eyes, they pick up more of the EOG activity critical for the classification of stage R. These channels are also close to the forehead region, whose lack of hair makes it a popular location for at-home polysomnography systems. Non-overlapping windows of 30 s of size (3000×2) were extracted,

On TUHab, the first minute of each recording was cropped to remove noisy data that occurs at the beginning of recordings. Longer files were also cropped such that a maximum of 20 minutes was used from each recording. Then, 21 channels that are common to all recordings were selected (Fp1, Fp2, F7, F8, F3, Fz, F4, A1, T3, C3, Cz, C4, T4, A2, T5, P3, Pz, P4, T6, O1 and O2). EEG channels were downsampled to 100 Hz and clipped at ±800 μV to mitigate the effect of large artifactual deflections in the raw data. Non-overlapping 6-s windows were extracted, yielding windows of size (600×21).

Finally, windows from both datasets with peak-to-peak amplitude below 1 μV were rejected. The remaining windows were normalized channel-wise to have zero-mean and unit standard deviation.

Results

The use of SSL tasks to learn useful EEG features from unlabeled data was investigated in a series of three experiments. First, SSL approaches were compared to fully supervised approaches based on deep learning or handcrafted features. Second, SSL-learned representations were explored to highlight clinically-relevant structure. Finally, in the last experiment, the impact of hyperparameter selection on pretext and downstream performance was studied.

SSL Models Learn Representations of EEG and Facilitate Downstream Tasks with Limited Annotated Data

Can the suggested pretext tasks enable SSL on clinical EEG data and mitigate the amount of labeled EEG data that is required in clinical tasks? To address this question, the pretext tasks were applied to two clinical datasets (PC18 and TUHab) and compared their downstream performance to the one of various established approaches such as fully supervised learning, while varying the number of labeled examples available.

Context and setup. Feature extractors h_{Θ} were trained using the different approaches (AE, RP, TS and CPC on unlabeled data) and then used to extract features. Following hyperparameter search (see Section “SSL pretext task hyperparameters strongly influence downstream task performance”), same-recording negative sampling was used on PC18 and across-recording negative sampling on TUHab. Features were also extracted with randomly initialized models. Downstream task performance was then evaluated by training linear logistic regression models on labeled examples, where the training set contains at least one and up to all existing labeled examples. Additionally, fully supervised models were trained directly on labeled data and random forests were trained on handcrafted features.

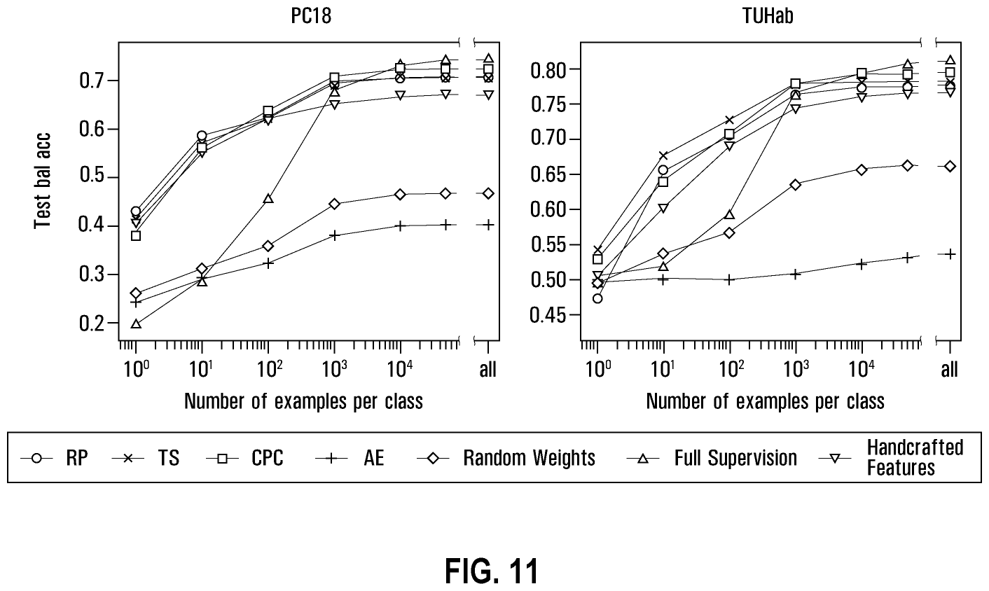

FIG. 11 illustrates impact of number of labeled examples per class on downstream performance. Feature extractors were trained with an autoencoder (AE), the relative positioning (RP), temporal shuffling (TS) and contrastive predictive coding (CPC) tasks, or left untrained (‘random weights’), and then used to extract features on PC18 and TUHab. Following a hyperparameter search, the same-recording negative sampling on PC18 and across-recording negative sampling on TUHab was used. Downstream task performance was evaluated by training linear logistic regression models on the extracted features for the labeled examples, with at least one and up to all existing labeled examples in the training set (‘All’). Additionally, fully supervised models were trained directly on labeled data and random forests were trained on handcrafted features. Results are the average of five runs with same initialization but different randomly selected examples. While more labeled examples led to better performance, SSL models achieved much higher performance than a fully supervised model when only few were available. Standard deviation (not shown) is high when few labeled examples are available, but decreases as more labeled examples are provided.

The impact of the number of labeled samples on downstream performance is presented in FIG. 11. First, when using SSL-learned features for the downstream tasks, important above-chance performance is observed across all data regimes: on PC18, models scored as high as 72.3% balanced accuracy (5-class, chance=20%) while on TUHab the highest performance was of 79.4% (2-class, chance=50%). These results demonstrate the ability of SSL to learn useful representations for downstream tasks. Second, the comparison suggests that SSL-learned features are competitive with other baseline approaches and can even outperform supervised approaches. On the PC18 sleep data (FIG. 11, “PC18”), one can observe that all three SSL pretext tasks outperformed alternative approaches including the fully supervised model and handcrafted features in most data regimes. The performance gap between SSL-learned features and full supervision was as high as 22.8 points when only one example per class was available. It remained in favor of SSL up to around 10,000 examples per class, where full supervision finally began to exceed SSL performance, however by a 1.6-3.5% margin only. Moreover, SSL outperformed the handcrafted features baseline over 100 examples per class, e.g., by up to 5.6 points for CPC. These results suggest two important implications: (1) pretext tasks can capture critical information for sleep staging, even though no sleep labels were available when learning representations and (2) these features can rival both human-engineered features and label-intensive full supervision.

Other baselines such as random weights and autoencoding obtained much lower performance, showing that learning informative features for sleep staging is not trivial and requires more sophistication than the inductive bias of a convolutional neural network alone or a pure reconstruction task. Interestingly, the poor performance of the AE can be attributed to its mean squared error loss. This encourages the model to focus on the signal's low frequencies, which, due to 1/f power-law dynamics have the largest amplitudes in bio-signals like EEG. Yet, low frequency signals only capture a small portion of the neurobiological information in EEG signals.

Next, SSL was applied to the task of pathology detection, where the two classes (“normal” and “abnormal”) are likely to be more heterogenous than the sleep staging classes. Again, SSL-learned features outperformed the baseline approaches in most data regimes: CPC outperformed full supervision when fewer than 10,000 labeled examples per class were available, while the performance gap between the two methods was on the order of 1% when all examples were available. Handcrafted features were also consistently outperformed by RP, TS and CPC, albeit by a smaller amount (e.g., 3.8-4.8 point difference for CPC). Again, the AE and random weights features could not compete with the other methods. Notably, the AE fared even worse on TUHab and downstream performance never exceeded 53.0%.

Taken together, the results demonstrate that the proposed SSL pretext tasks were general enough to enable two fundamentally different types of EEG classification problems. All SSL tasks systematically outperformed or equaled other approaches in low-to-medium labeled data regimes and remained competitive in a high labeled data regime.

SSL Models Capture Physiologically and Clinically Meaningful Features

While SSL-learned features yielded competitive performance on sleep staging and pathology detection tasks, it is unclear what kind of structure was captured by SSL. To address this, the embeddings were examined by analyzing their relationship with the different annotations and meta-data available in clinical datasets. For this purpose, the 100-dimensional embeddings obtained on PC18 and TUHab were projected onto a two-dimensional representation using Uniform Manifold Approximation and Projection (UMAP) and using models as identified in the section “SSL models learn representations of EEG data and facilitate downstream tasks with limited annotated data”. This allows a qualitative analysis of the local and global structure of the SSL-learned features.

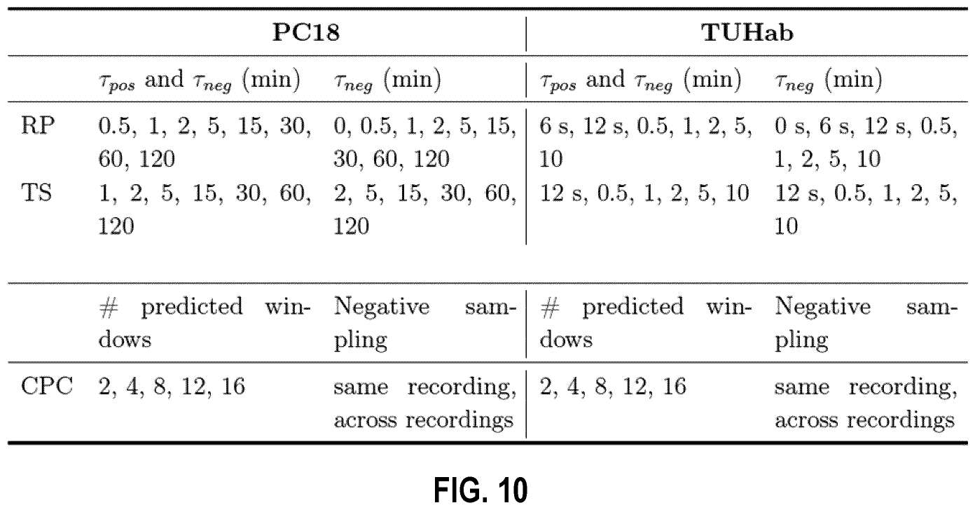

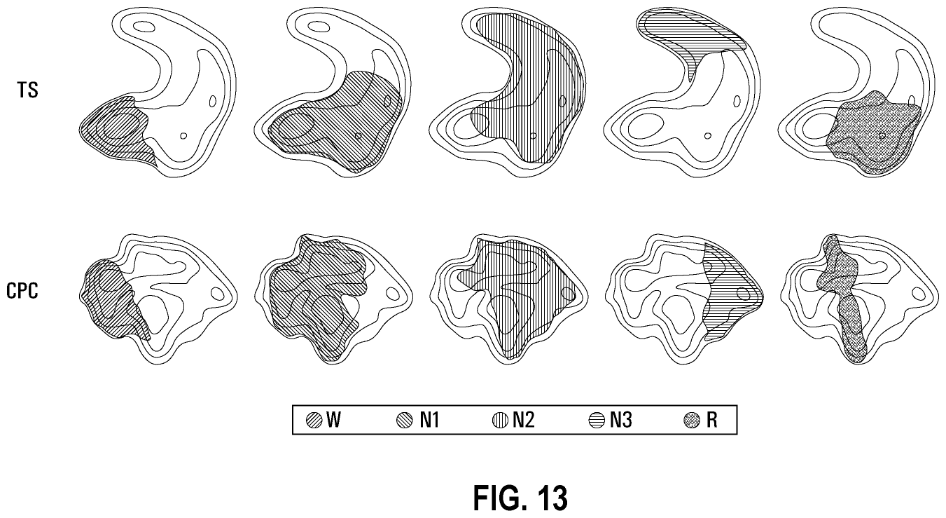

FIG. 13 illustrates UMAP visualization of SSL features on the PC18 dataset. The subplots show the distribution of the 5 sleep stages as scatterplots for TS (first row) and CPC (second row) features. Contour lines correspond to the density levels of the distribution across all stages and are used as visual reference. Finally, each point corresponds to the features extracted from a 30-s window of EEG by the TS and CPC embedders with the highest downstream performance as identified in the section “SSL pretext task hyperparameters strongly influence downstream task performance” and in FIG. 10. All available windows from the train, validation and test sets of PC18 were used. In both cases, there is clear structure related to sleep stages although no labels were available during training.

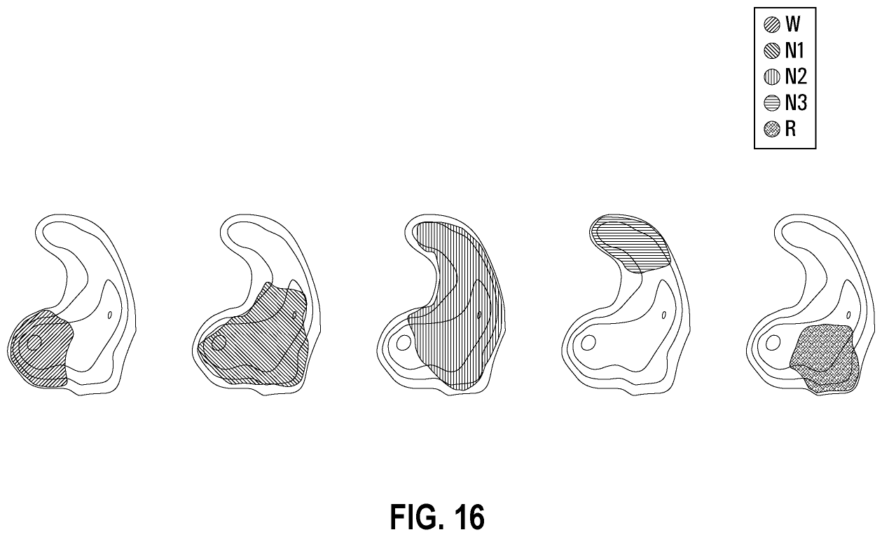

FIG. 16 illustrates UMAP visualization of SSL features on the PC18 dataset. The subplots show the distribution of the 5 sleep stages as scatterplots for RP features. Contour lines correspond to the density levels of the distribution across all stages and are used as visual reference. Finally, each point corresponds to the features extracted from a 30-s window of EEG by the RP embedders with the highest downstream performance. All available windows from the train, validation and test sets of PC18 were used.

Results on sleep data are shown in FIG. 13 and FIG. 16. Referring to FIG. 13, a structure that closely follows the different sleep stages can be noticed in the embeddings of PC18 obtained with TS and CPC. By looking at the distribution of examples from the different stages, clear groups emerge. They not only correspond to the labeled sleep stages, but they are also sequentially arranged: moving from one end of the embedding to another, a trajectory can be drawn that passes through W, N1, N2 and N3 sequentially. Stage R, finally, mostly overlaps with N1.

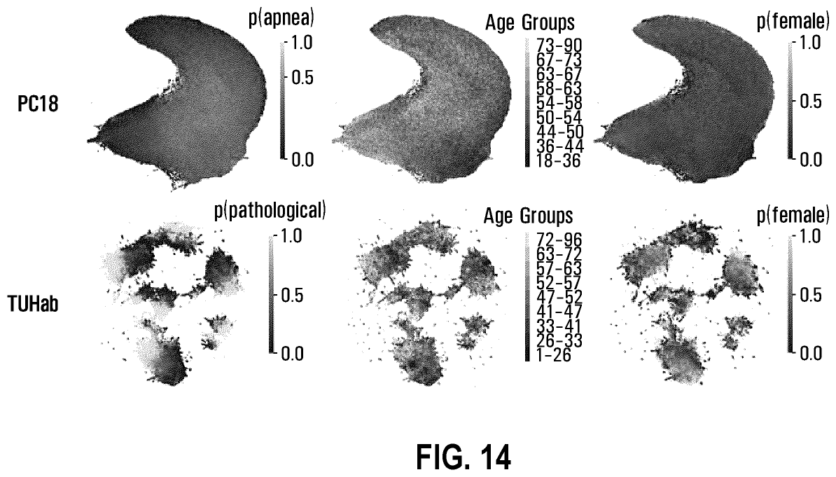

FIG. 14 illustrates structure learned by the embedders trained on the TS task. The models with the highest downstream performance, as identified in the section “SSL pretext task hyperparameters strongly influence downstream task performance” and in FIG. 10, were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with the SSL tasks capture physiologically-relevant structure, such as pathology, age, apnea and gender.

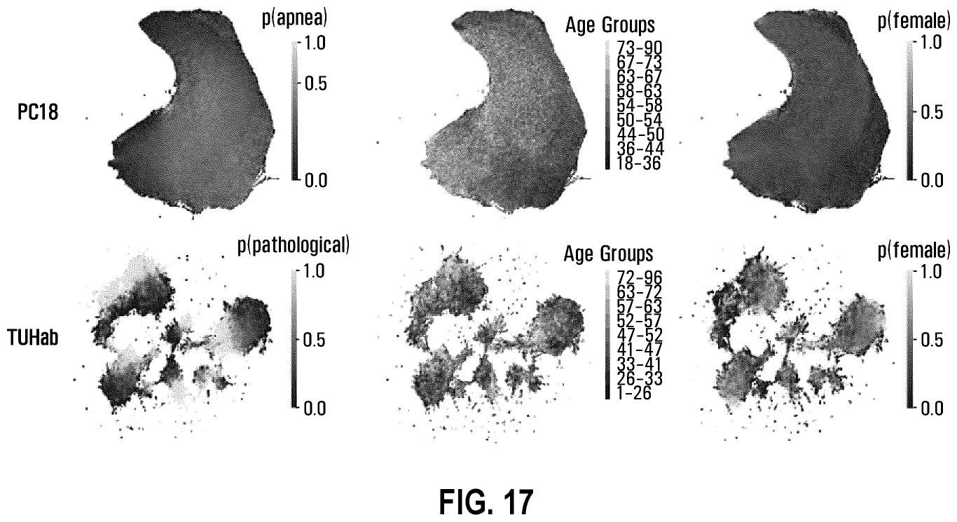

FIG. 17 illustrates structure learned by the embedders trained on the RP task. The models with the highest downstream performance, as identified in the section “SSL pretext task hyperparameters strongly influence downstream task performance” and in FIG. 10, were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with the SSL tasks capture physiologically-relevant structure, such as pathology, age, apnea and gender.

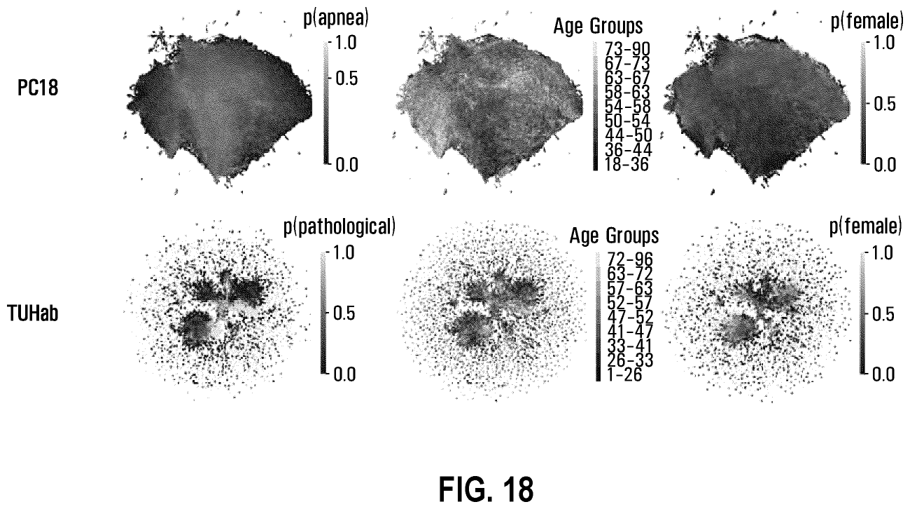

FIG. 18 illustrates structure learned by the embedders trained on the CPC task. The models with the highest downstream performance, as identified in the section “SSL pretext task hyperparameters strongly influence downstream task performance” and in FIG. 10, were used to embed the combined train, validation and test sets of the PC18 and TUHab datasets. The embeddings were then projected to two dimensions using UMAP and discretized into 500×500 “pixels”. For binary labels (“apnea”, “pathological” and “gender”), the probability was visualized as a heatmap, i.e., the color indicates the probability that the label is true (e.g., that a window in that region of the embedding overlaps with an apnea annotation). For age, the subjects of each dataset were divided into 9 quantiles, and the color indicates which group was the most frequent in each bin. The features learned with the SSL tasks capture physiologically-relevant structure, such as pathology, age, apnea and gender.

The largest sources of variation in sleep EEG data are likely linked to changes in sleep stages and the corresponding microstructure (e.g., slow waves, sleep spindles, etc.). Can other sources of variation be expected to also be visible in the embeddings? To address this question, clinical information available in PC18 was inspected along with the embeddings: apnea events and subject age. The results are presented in the first row of FIG. 14 for TS and FIG. 17 for RP and FIG. 18 for CPC. Similar conclusions apply to all three methods. First, apnea-related structure can be seen in the middle of the embeddings, overlapping with the area where stage N2 was prevalent (first column of FIG. 14). At the same time, very few apnea events occurred at the extremities of the embedding, for instance over W regions, naturally, but also over N3 regions. Although this structure likely reflects the correlation between sleep stages, age and actual apnea-induced EEG patterns, this nonetheless shows the potential of SSL to learn features that relate to clinical phenomena. Second, age structure was revealed in at least two distinct ways in the embeddings (second column of FIG. 14). The first is related to sleep macrostructure, i.e., the sequence of sleep stages and their relative length. Indeed, younger subjects were predominant over the R stage region, while older subjects were more frequently found over the W region. This is in line with phenomena such as increased sleep fragmentation and sleep onset latency in older individuals, as well as a subtle reduction in REM sleep with age. Concurrently, sleep microstructure is also observed in the embeddings. For instance, looking at N2-N3 regions, older age groups are more likely to be found in the leftmost side of the blob, while younger subjects are more likely to be found on its rightmost side. This suggests there is a difference between the characteristics of N2-N3 sleep across age groups, e.g., related to sleep spindles. Finally, there is also gender-related structure, with discernible low and high probability regions in the embeddings (third column of FIG. 14)

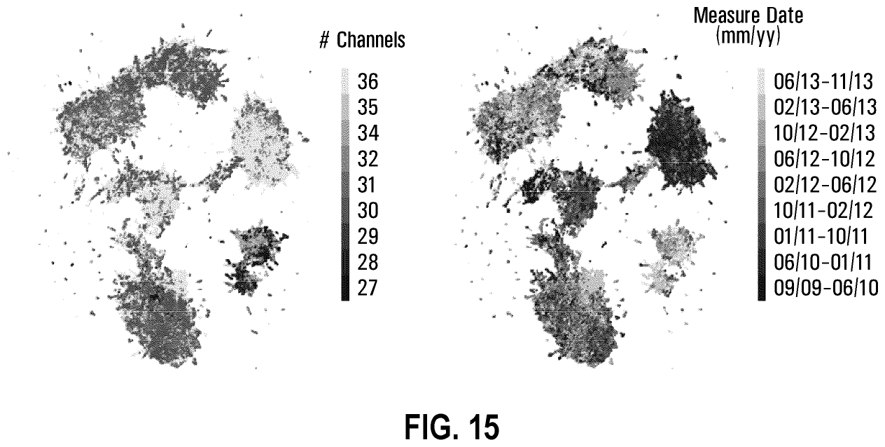

FIG. 15 illustrates structure related to the original recording's number of EEG channels and measurement date in TS-learned features on the entire TUHab dataset. The overall different number of EEG channels and measurement date in each cluster shows that the cluster-like structure reflects differences in experimental setups.

Can this clinically-relevant structure also be learned on a different type of EEG recording? A similar analysis was conducted for TUHab, this time focusing on pathology, age and gender. Results are shown in the second row of FIG. 14, FIG. 17, and FIG. 18. The embeddings exhibited a primary multi-cluster structure, with similar gradient-like structure inside each cluster. For instance, pathology-related structure was clearly exposed in the two embeddings (column 1), with an increasing probability of the EEG being abnormal when moving from one end of the different clusters to the other. Likewise, an age-related gradient emerged inside each cluster (column 2), in a similar direction as the pathology gradient, while there is also a gender-associated gradient that appeared orthogonal to the first two (last column). What do the different clusters actually represent? Experimental setup-related labels (the original number of EEG channels and the measurement date of each recording) are plotted in FIG. 15. Each cluster was predominantly composed of examples with a given number of channels and with a specific range of measurement dates. This might suggest that the SSL tasks have partially learned the noise introduced by data collection. For example, the TUHab dataset was collected over many years across different sites, by different EEG technicians and with various EEG devices. Most likely, the impact of this noise in the embedding could be mitigated by using more aggressive preprocessing (e.g., bandpass filtering) or by sampling negative examples within recordings from the same cohort.

In conclusion, this experiment showed that SSL can learn to encode clinically-relevant structure such as sleep stages, pathology, age, apnea and gender information from EEG data, while revealing interactions (such as young age and REM sleep), without any access to labels.

SSL Pretext Task Hyperparameters Strongly Influence Downstream Task Performance

How should the various SSL pretext task hyperparameters be tuned to fully make use of self-supervision in clinical EEG tasks? The following describes how the hyperparameters of the models used in the experiments above were tuned and the impact of some key hyperparameters on downstream performance.

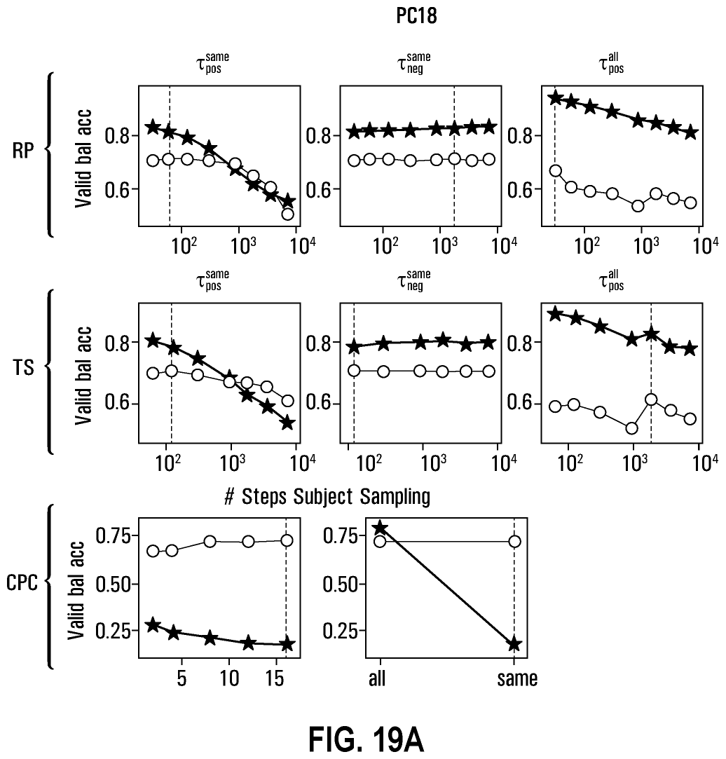

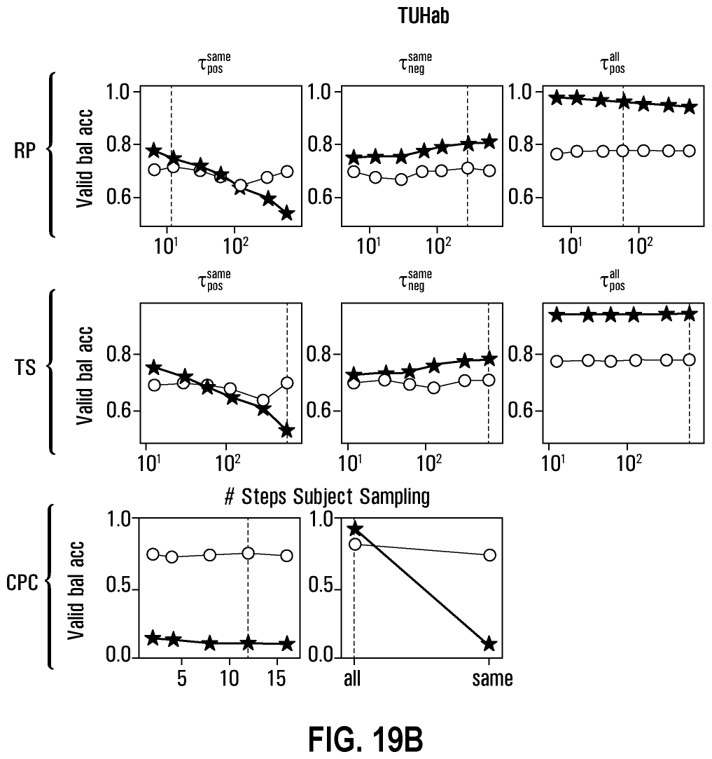

FIG. 19 illustrates impact of principal hyperparameters on pretext (black, star) and downstream (white, circle) task performance, measured with balanced accuracy on the validation set on (A) PC18 and (B) TUHab. Each row corresponds to a different SSL pretext task. For both RP and TS, the hyperparameters that control the length of the positive and negative contexts (τₚₒₛ, τ_{neg}, in seconds) were varied; the exponent “same” or “all” indicates whether negative windows were sampled across the same recording or across all recordings, respectively. For CPC, the number of predicted windows and the type of negative sampling was varied. Finally, the best hyperparameter values in terms of downstream task performance are emphasized using vertical dashed lines.

To benchmark different pretext tasks across datasets, the performance of the pretext and downstream tasks were tracked across different choices of hyperparameters (see Section “Hyperparameter search procedure” for a complete description of the search procedure). The comparison is depicted in FIG. 19A and FIG. 19B. The analysis suggests that the pretext tasks performed significantly above chance level on all datasets: RP and TS reached a maximum performance of 98.0% (2-class, chance=50%) while CPC yielded performances as high as 95.4% (32-class, chance=3.1%). On the downstream tasks, SSL-learned representations always performed above chance as reported in Section “SSL models learn representations of EEG and facilitate downstream tasks with limited annotated data”. Interestingly though, configurations with high pretext performance did not necessarily lead to high downstream performance, which highlights the necessity of appropriate hyperparameter selection.

In the next step, the influence of the different hyperparameters on each pretext task (rows of FIG. 19A and FIG. 19B) was examined to identify optimal configurations. First, the focus was on the same-recording negative sampling scenario, in which negative examples are sampled from the same recording as the anchor window(s). With RP, increasing τₚₒₛ always made the pretext task harder. This is expected since the larger the positive context, the more probable it is to get positive example pairs that are composed of distant (and thus potentially dissimilar) windows. On sleep data, a plateau effect was noticed: the downstream performance was more or less constant below τₚₒₛ=20 min, suggesting EEG autocorrelation properties might be changing at this temporal scale. Although this phenomenon did not appear clearly on TUHab, downstream performance decreased above τₚₒₛ=30 s, and then increased again after τₚₒₛ=2 min. On the other hand, varying τ_{neg} given a fixed τₚₒₛ did not have a consistent or significant influence on downstream performance, although larger τ_{neg} generally led to easier pretext tasks.

Do these results hold when negative windows are sampled across all recordings? Interestingly, the type of negative sampling has a considerable effect on downstream performance (column 3 of FIG. 19A and FIG. 19B). On sleep staging, downstream performance dropped significantly and degraded faster as τₚₒₛ was increased, while the opposite effect could be seen on the pathology detection task (higher, more stable performance). This effect might be explained by the nature of the downstream task: in sleep staging, major changes in the EEG occur inside a given recording, therefore distinguishing between windows of a same recording is key to identifying sleep stages. On the other hand, in pathology detection, each recording is given a single label (“normal” or “pathological”) and so being able to know whether a window comes from the same recording (necessarily with the same label) or from another one (possibly with the opposite label) intuitively appears more useful. In other words, the distribution that is chosen for sampling negative examples determines the kind of invariance that the network is forced to learn. Overall, similar results hold for TS.

As for CPC, a similar analysis shows that while increasing the number of windows to predict (“number of steps”) made the pretext task more difficult, predicting further ahead in the future helped the embedder learn a better representation for sleep staging (bottom row of FIG. 19A and FIG. 19B). Pretext performances of around 20% might seem low, however they are in fact significantly higher than chance level (3.1%) on this 32-class problem. Remarkably, the type of negative sampling had a minor effect on downstream performance on sleep data (71.6 vs. 72.2% bal acc), but had a considerable effect on pathology detection (74.1 vs. 80.4%), akin to RP and TS above. This result can show that subject-specific negative sampling led to the highest downstream performance on a phoneme classification downstream task.

In this last experiment, it is confirmed that SSL pretext tasks are not trivial, and that certain pretext task hyperparameters have a measurable impact on downstream performance.

Hyperparameter Search Procedure

FIG. 10 is a table of SSL pretext task hyperparameter values considered in training. Bold face indicates values that led to the highest downstream task performance.

The hyperparameter search was carried out using the following steps. First, embedders h_{Θ} were independently trained on the RP, TS and CPC tasks. The parameters of h_{Θ} were then frozen, and the different h_{Θ} were used as feature extractors to obtain sets of 100-dimensional feature vectors from the original input data. Finally, linear logistic regression classifiers were trained to perform the downstream tasks given the extracted features. The principal pretext task hyperparameters were further varied to understand their impact on both pretext and downstream task performance (see FIG. 10). In both cases, the balanced accuracy was compared on the validation set. For RP and TS, attention was focused on τₚₒₛ and τ_{neg}, which are used to control the size of the positive and negative contexts when sampling pairs or triplets of windows. As a first step, the values of τₚₒₛ and τ_{neg} were varied jointly, i.e., τₚₒₛ=τ_{neg}, to avoid sampling “confusing” pairs or triplets of windows which could come from either the positive or negative classes. The best value was then used to set τₚₒₛ, and a sweep over different τ_{neg} values was carried out. In a second step, τ_{neg} was fixed such that it encompassed all recordings, i.e., negative windows were uniformly sampled from any recording in the dataset instead of being limited to the recording which contains the anchor window. τₚₒₛ was again varied with this second negative sampling strategy. For CPC, the impact of the number of predicted windows (“#steps”) was studied and, as for RP and TS, the type of negative sampling (“same-recording” vs. “across-recordings”). Again, the number of predicted windows was varied and the best value reused to compare negative sampling strategies.

### Discussion

Self-supervised learning (SSL) has been disclosed herein as a way to learn representations on EEG data. Specifically, two SSL tasks are designed to capture structure in EEG data, relative positioning (RP) and temporal shuffling (TS) and adapted a third approach, contrastive predictive coding (CPC), to work on EEG data. In contrast to other approaches, these methods do not require manual feature extraction, additional demographic information or a pretraining step, and do not rely on data augmentation transforms that have yet to be defined on EEG. As demonstrated, these tasks can be used to learn generic features which capture clinically relevant structure from unlabeled EEG, such as sleep micro- and macrostructure and pathology. Moreover, a rigorous comparison of SSL methods to traditional unsupervised and supervised methods on EEG was performed, and showed that downstream classification performance can be significantly improved by using SSL, particularly in low-labeled data regimes. These results hold for two large-scale EEG datasets comprising sleep and pathological EEG data, both with thousands of recordings.

Using SSL to Improve Performance in Semi-Supervised Scenarios

Conveniently, it is shown that SSL can be used to improve downstream performance when a lot of unlabeled data is available but labelled data is scarce, i.e., in a semi-supervised learning scenario. For instance, CPC-learned features outperformed fully supervised learning on sleep data by about 20% when only one labeled example per class was available. Similarly, on the pathology detection task an improvement of close to 15% was obtained with SSL when only 10 labeled examples per class were available. In practice, SSL has the potential to become a common tool for boosting classification performance when annotations are expensive, a common scenario when working with biosignals such as EEG.

While the SSL pretext tasks included in this work are applicable to multivariate time series in general, their successful application does require adequate recording length and dataset size. First, the EEG recordings need to be sufficiently long so that a reasonable number of windows can be sampled given the positive and negative contexts and the window length. For clinical recordings, this is typically not an issue: sleep monitoring and pathology screening procedures both produce recordings of tens of minutes to many hours, which largely suffice. It is noteworthy that while the proposed SSL approaches have been developed using clinical data, they could be readily applied to event-related EEG protocols, such as those encountered in cognitive psychology or brain-computer interfacing experiments. Indeed, SSL could be applied to an entire recording without explicitly taking into consideration the known events (e.g. stimuli, behavioral responses). This critically depends on the availability of continuous recordings (rather than epoched data only, as is the case in some public EEG datasets) including resting state baselines and between-trial segments. Other stimulus-presentation based protocols might also be used when entire recordings are available (rather than event-related windows only). Second, the current results may suggest large datasets are necessary to enable SSL on EEG as analyses were based on two of the largest publicly available EEG datasets with thousands of recordings each. However, similar results hold on much smaller datasets containing fewer than 100 recordings. As long as the unlabeled dataset is representative of the variability of the test data, representations that are useful for the pretext task should be transferable to a related downstream task.

One might argue that the observed performance benefit of SSL is minor as compared to supervised approaches when the number of samples is moderate to large. Is investing in the development of SSL-based EEG-analysis worth the effort? It is important to highlight that the results herein present a proof of concept that opens the door to further developments, which may lead to substantial improvements in performance. Experiments disclosed herein are limited to the linear evaluation protocol, where the downstream task is carried out by a linear classifier trained on SSL-learned features, in order to focus on the properties of the learned representations. Finetuning the parameters of the embedders on the downstream task could further improve downstream performance results.

Preliminary experiments (not shown) suggested that a 3 to 4-point improvement can be obtained in some data regimes when finetuning the embedders and that performance with all data points is as high as with a purely supervised model. However, using nonlinear classifiers (here, random forests) on SSL-learned features did not improve results, suggesting downstream tasks disclosed herein might be sufficiently close to the pretext task that the relevant information is already linearly accessible.

Generally speaking, self-supervision also presents interesting opportunities to improve model performance as compared to purely supervised approaches. Another potential opportunity to improve downstream performance consists of using larger deep learning architectures. Due to their sampling methodology, most SSL tasks can “create” a combinatorially number of distinct examples going beyond the number of labeled examples typically available in a supervised task. For instance, on PC18, the training set contained close to 5×10⁵ labeled examples while for self-supervised tasks embedders disclosed herein were trained on more than twice that number of examples (to fit computational time requirements), though more could have been easily sampled. The much higher number of available examples in SSL opens the door to using much larger deep neural networks which require much larger sample sizes. Given the relatively shallow architectures currently used in deep learning and EEG research (on which choice of architectures was based), SSL could be key to training deeper models and improving upon current state of the art on various EEG tasks.

Second, in most applications, it is desirable that a trained model generalizes across individuals, including individuals not in the training set. Therefore, many classification approaches rely on subject-dependent training to reach optimal performance. This however comes at the cost of requiring subject-specific labeled data. SSL is likely to help overcome this challenge. Indeed, given larger (unlabeled) datasets, SSL pretraining can improve the diversity of examples captured by the learned feature space and, in doing so, act as a strong regularizer against overfitting on the individuals of the training set.

Finally, although an increasing number of deep learning-EEG studies choose raw EEG as input to their neural networks, handcrafted feature representations are still used in a large portion of recent papers. This raises the question of what optimal handcrafted features are for a specific task. SSL brings an interesting solution to this problem, as the features it learns can capture important physiological parameters from raw data, and their reuse in a deep neural network is straightforward (e.g. as weight initialization). Although handcrafted features are inherently more interpretable, model inspection techniques show that learned features can be meaningfully interpreted as well. Therefore, given the potential of SSL-learned features to capture true statistical sources, SSL might close the gap between raw EEG- and handcrafted features-based approaches.

Sleep-Wakefulness Continuum and Inter-Rater Reliability

It has been demonstrated that the embeddings learned with SSL capture clinically-relevant information. Sleep, pathology, age and gender information appeared as clear structure in the learned feature space (see FIG. 13 and FIG. 14). The variety of metadata that is visible in the embeddings highlights the capacity of the proposed SSL tasks to uncover important factors of variation in noisy biosignal data such as EEG in a purely unsupervised manner. Critically though, this structure is not discrete, but continuous. Indeed, sleep stages are not cleanly separated into five clusters (or two clusters for normal and abnormal EEG), but instead the embeddings display a smooth continuum of sleep-wakefulness (or of normal-abnormal EEG). Is this gradient-like structure meaningful, or is it a mere artifact of experimental setup? The continuous nature of SSL-learned features may be inherent to the neurophysiological phenomena under study. Conveniently, this offers interesting opportunities to improve the analysis of physiological data.

While sleep EEG is routinely divided into discrete stages to be analyzed, its true nature is likely continuous. For instance, the concept of sleep stages and the taxonomy that is known today is the product of incremental standardization efforts in the sleep research community. Although this categorization is convenient, critics still stress the limitations of stage-based systems under the evidence of sub-stages of sleep and the interplay between micro- and macrostructure. Moreover, even trained experts using the same set of rules do not perfectly agree on their predictions, showing that the definition of stages remains ambiguous: an overall agreement of 82.6% was obtained between the predictions of more than 2,500 trained sleep scorers (even lower for N1, at 63.0%). Consequently, could a better representation of sleep EEG, such as the one learned with self-supervision, alleviate some of these challenges? While previous research suggests sleep might indeed be measured using a continuous metric derived from a supervised machine learning model or a computational mean-field model, embodiments disclosed herein demonstrate that the rich feature space learned with SSL can simultaneously capture sleep-related structure and variability caused by age and apnea. Importantly, the data-driven nature of the SSL representation alleviates the subjectivity of manual sleep staging. Overall, this suggests SSL-learned representations could provide more fine-grained information about the multiple factors at play during sleep and, in doing so, enable a more precise study of sleep.

Similarly, many EEG pathologies are described by a clinical classification system which defines discrete subtypes of diseases or disorders, e.g., epilepsy and dementia. As for sleep EEG, inter-rater agreement is limited. This suggests that there is an opportunity for these pathologies to be interpreted as a continuum as well. Although the pathology detection downstream task disclosed herein was a simple binary classification task, the clear pathology-associated gradient captured by SSL pretext tasks could be used to characterize the different types and subtypes of pathologies contained in the dataset more precisely (see FIG. 14). Ultimately, the data-driven feature space obtained with SSL might aid in the study and comparison of different neuropathologies and EEG patterns in general.

Finding the Right Pretext Task for EEG

With the large number of self-supervised pretext tasks one can think of, and the even larger number of possible EEG downstream tasks, how can a combination of pretext task and hyperparameters be chosen for a given setting? To answer this question, many more empirical experiments will have to be conducted on EEG data. However, the results presented here give some insight as to what may be important to consider. In this work, pretext tasks were developed that proved effective on two different classification problems by using a combination of (1) prior knowledge about EEG time series, (2) assumptions about the statistical structure of the features to be learned, (3) thorough hyperparameter search and (4) computational considerations.

First, the relative positioning (RP) and temporal shuffling (TS) tasks were tailed to EEG by relying on prior knowledge about EEG. These tasks were designed specifically with the structure of sleep data in mind. Indeed, sleep EEG signals have a clear temporal structure originating from the succession of sleep stages during the night. This means that two windows that are close in time have a high probability of sharing the same sleep stage annotation and statistical structure. Therefore, learning to differentiate close-by from faraway windows may be related to learning to differentiate sleep stages. Similar approaches are applied in the computer vision, however rely on properties of natural images that generally do not hold for EEG. For instance, whereas two EEG windows xₜ and xₜ, that are close in time likely look alike, there is typically no physiological information in these windows that would allow to determine whether t<t′ or t>t′. Exceptions would include transitions between sleep stages that are more likely than others, such as from lighter to deeper sleep stages; however these transitions occur rarely during the night; more often a back-and-forth between sleep stages is observed. Therefore, tasks that rely on proximity rather than absolute positioning appear to be a better match for EEG. CPC is included in experiments disclosed herein as it is a natural extension of RP and TS and has led to promising results on other kinds of data.

Second, assumptions about the statistical structure of the latent factors or features to recover was used to further support choice of tasks. For instance, given its similarity with permutation contrastive learning (PCL, a self-supervised method for performing nonlinear ICA), RP likely relies on the general temporal dependencies (including autocorrelations) of EEG signals to recover informative features. PCL can be obtained by setting RP's τₚₒₛ to be the length of a single window and τ_{neg} to 0. Incidentally, the optimal values of τₚₒₛ and τ_{neg} were found to be relatively small on the datasets considered, suggesting hyperparameters close to those of PCL are optimal. Since TS and CPC can both be seen as extensions of the RP task with more elaborate sampling strategies and contrastive procedures (see section “Self-supervised learning pretext tasks for EEG”, above), all three tasks might effectively rely on similar structure to discover features.

Third, the careful selection of pretext task hyperparameters was essential to selecting the right pretext task configuration. For instance, RP, TS and CPC often yielded very similar downstream task performance once the best hyperparameters were selected. Out of the main pretext task hyperparameters, the negative sampling strategy proved to be especially important to tune (see section “SSL models learn representations of EEG data and facilitate downstream tasks”, above). Indeed, sleep staging benefited from same-recording negative sampling whereas pathology detection instead worked better when across-recording negative sampling was used. Interestingly, this appears to be the unique change to RP, TS and CPC that is required to compete with purely supervised approaches on the pathology detection downstream task, although RP and TS were initially designed for capturing intra-recording sleep structure. Thus, negative sampling hyperparameters might be among the most critical hyperparameters to tune, as they can be used to develop invariances to particular structure that is not desirable, e.g., intra-recording changes or measurement-site effects (FIG. 15). Ultimately, the fact that all three pretext tasks could reach similar downstream performance suggests self-supervision was able to uncover fundamental information likely related to physiology.

Finally, computational requirements and architecture-specific constraints are important to consider when choosing a pretext task. Given the similar downstream performance yielded by each pretext task after hyperparameter selection, RP might be preferred over CPC if they can reach similar performance. Indeed, CPC has more hyperparameters than RP and TS (i.e., number of context windows, of windows to be predicted and of negative examples; architecture of the autoregressive embedder g_{AR}) and requires additional forward and backward passes through the embedder h_{Θ} at training time. However, CPC's autoregressive encoder g_{AR} could yield better features for some tasks with larger-scale dependencies, e.g., sleep staging. Indeed, deep learning for automated polysomnography show improved performance using larger-scale temporal information. This aggregation of window-level information is already part of a CPC model and therefore the pretrained g_{AR} could be reused directly. Preliminary results (not shown) suggest these autoregressive features can substantially improve downstream performance on both the sleep staging and pathology detection tasks.

Although the proposed tasks proved successful, many other pretext tasks could have been designed based on an understanding of EEG. For instance, a transformation discrimination task was applied to ECG data could be adapted for EEG. Similarly, nonlinear ICA-derived frameworks such as time contrastive learning and generalized contrastive learning could be used to explicitly leverage nonstationnarity or other structure present in EEG signals.

Example experiments had fixed training hyperparameters across data regimes.

Deep neural networks can easily overfit the training data when few examples are available, which can negatively impact generalization. Example ways of addressing overfitting include increasing the strength of regularization such as dropout and weight decay and performing early stopping. Given the computational requirements of training neural networks on large EEG datasets, in example experiments the training hyperparameters of the fully supervised models (i.e., learning rate, batch size, dropout, weight decay) were fixed and the same values were reused across all data regimes. As a result, the fully supervised models typically stopped learning after only a few epochs, although they might have been able to train longer with different hyperparameters. The impact of various training hyperparameter settings were tested on a subset of the models and it was observed that even though training can be slightly improved by changing hyperparameters, this effect is not strong enough to change any of the conclusions herein (results not shown).

In example embodiments, the hyperparameter search was limited to pretext task hyperparameters in experiments. However, architecture hyperparameters (e.g., number of convolutional channels, dimensionality of the embedding, number of layers, etc.) can also play a critical role in achieving high performance using SSL. Sticking to a single fixed architecture for all models and data regimes means that these improvements—which could help bridge (or widen) the gap between SSL methods and the various baselines—were not taken into account herein.

Embodiments disclosed herein introduce self-supervision as a representation learning paradigm for EEG. This may be the first presentation of sleep staging results on PC18. Nonetheless, downstream performance would most likely improve significantly by aggregating temporal windows as was shown on other datasets. Similarly, on TUHab, simpler approaches were reused instead of the best performing models. Moreover, (1) the cropped decoding approach presented was not used, (2) a simpler normalization methodology was used (z-score instead of exponential moving average normalization) and (3) the train/validation data split was different. Together, these differences explain the small drop in performance between these state-of-the-art methods and the results reported here.

In this work, SSL approaches were introduced to learn representations on EEG data and it was shown that they could compete with and sometimes outperform traditional supervised approaches on two large clinical tasks. The features learned by SSL displayed a clear structure in which different physiological quantities were jointly encoded. This validates the potential of self-supervision to capture important physiological information even in the absence of labeled data. SSL may also be used with other kinds of EEG recordings and tasks, such as regression. Ultimately, developing a better understanding of how a pretext task can be designed to target a specific kind of EEG structure will be useful in establishing self-supervision as an example component of any EEG analysis pipeline.

The foregoing described non-limiting, exemplary research.

### Example Implementation

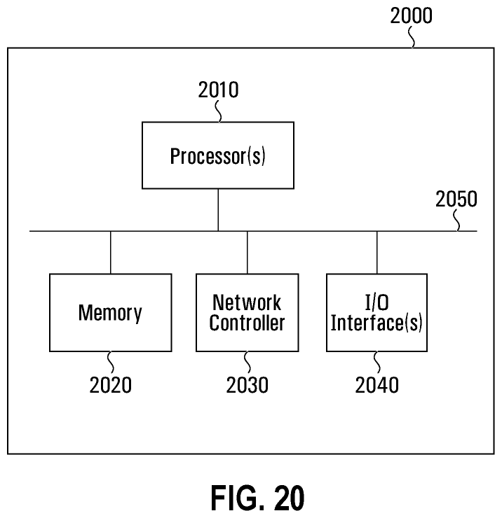

FIG. 20 is a block diagram of example hardware and software components of a computing device, according to an embodiment.

Techniques such as systems and methods disclosed herein may be implemented as software and/or hardware, for example, in a computing device 2000.

As illustrated, computing device 2000 includes one or more processor(s) 2010, memory 2020, a network controller 2030, and one or more I/O interfaces 2040 in communication over bus 2050.

Processor(s) 2010 may be one or more Intel x86, Intel x64, AMD x86-64, PowerPC, ARM processors or the like.

Memory 2020 may include random-access memory, read-only memory, or persistent storage such as a hard disk, a solid-state drive or the like. Read-only memory or persistent storage is a computer-readable medium. A computer-readable medium may be organized using a file system, controlled and administered by an operating system governing overall operation of the computing device.

Network controller 2030 serves as a communication device to interconnect the computing device with one or more computer networks such as, for example, a local area network (LAN) or the Internet.

One or more I/O interfaces 2040 may serve to interconnect the computing device with peripheral devices, such as for example, keyboards, mice, video displays, and the like. Such peripheral devices may include a display of device 2000. Optionally, network controller 2030 may be accessed via the one or more I/O interfaces.

Software instructions are executed by processor(s) 2010 from a computer-readable medium. For example, software may be loaded into random-access memory from persistent storage of memory 2020 or from one or more devices via I/O interfaces 2040 for execution by one or more processors 2010. As another example, software may be loaded and executed by one or more processors 2010 directly from read-only memory.

The above described embodiments are intended to be illustrative only and in no way limiting. The described embodiments are susceptible to many modifications of form, arrangement of parts, details and order of operation. The disclosure is intended to encompass all such modification within its scope, as defined by the claims.

1. A system for training a neural network to classify bio-signal data, the system comprising:
a memory configured to store training bio-signal data from one or more subjects, wherein the training bio-signal data comprises labeled training bio-signal data and unlabeled training bio-signal data;
a training computing apparatus configured to:
receive the training bio-signal data from the memory;
define one or more sets of time windows within the training bio-signal data, each set comprising a first anchor window and a sampled window;
for at least one set of the one or more sets:
determine a determined set representation based in part on the relative position of the first anchor window and the sampled window;
extract a feature representation of the first anchor window and a feature representation of the sampled window using an embedder neural network;
aggregate the feature representations using a contrastive module;
predict a predicted set representation using the aggregated feature representations;
wherein the set representation denotes likely label correspondence between the first anchor window and the sampled window; and
update trainable parameters of the embedder neural network to minimize a difference between the determined set representation of the at least one set and the predicted set representation of the at least one set; and
label the unlabeled training bio-signal data using a classifier, the labeled training bio-signal data, and the embedder neural network.

2. The system of claim 1 further comprising:
a bio-signal sensor configured to receive user bio-signal data from a user;
a classifying computing apparatus configured to:
receive the embedder neural network from the training computing apparatus;
receive the user bio-signal data from the bio-signal sensor; and
label the user bio-signal data using the embedder neural network.

3. (canceled)

4. (canceled)

5. The system of claim 1, wherein:
the one or more sets of time windows comprise one or more pairs of time windows;
the at least one set of the one or more sets comprises at least one pair of the one or more pairs;
the training computing apparatus determines the determined set representation based in part on the relative position of the first anchor window and the sampled window by:
defining a positive context region and a negative context region surrounding the first anchor window; and
determining if the sampled window is within the positive context region or the negative context region.

6. (canceled)

7. The system of claim 1, wherein:
the one or more sets of time windows comprise one or more triplets of time windows, each triplet further comprising a second anchor window, wherein the second anchor window is within a positive context region surrounding the first anchor window;
the at least one set of the one or more sets comprises at least one triplet of the one or more triplets;
the training computing apparatus determines the determined set representation based in part on the relative position of the first anchor window and the sampled window by:
determining a temporal order of the first anchor window, the sampled window, and the second anchor window;
the extract the feature representation of the first anchor window and a feature representation of the sampled window using an embedder neural network further comprises extracting a feature representation of the second anchor window.

8. (canceled)

9. The system of claim 1, wherein:
the first anchor window comprises a series of consecutive anchor windows;
the sampled window comprises a series of consecutive sampled windows, wherein the series of consecutive sampled windows is adjacent to the series of consecutive anchor windows;
the set further comprises a set of negative sample windows;
the training computing apparatus determines the determined set representation based in part on the relative position of the first anchor window and the sampled window by determining that a given sampled window is in the series of sampled windows;
the extract the feature representation of the first anchor window and a feature representation of the sampled window using an embedder neural network comprises:
extracting a feature representation of each anchor window of the series of consecutive anchor windows;
extracting a feature representation of each sampled window of the series of consecutive sampled windows; and
extracting a feature representation of each negative sample window of the set of negative sample windows;
the aggregate the feature representations comprises:
embedding the feature representation of each anchor window of the series of anchor windows using an autoregressive embedder;
aggregating the embedded anchor series, a given feature representation of a given sampled window of the series of sampled windows, and one or more given feature representations of one or more given negative sample windows of the set of negative sample windows;
the predict the predicted set representation comprises predicting which of the given feature representations corresponds to the given feature representation of the sampled window of the series of sampled windows.

10. (canceled)

11. (canceled)

12. (canceled)

13. (canceled)

14. (canceled)

15. (canceled)

16. The system of claim 2, wherein:
the training computing apparatus comprises a server configured to upload the embedder neural network and the classifier to the classifier computing apparatus.

17. (canceled)

18. The system of claim 1, wherein:
the classifier comprises a classifier neural network; and
the update trainable parameters comprises updating trainable parameters of the classifier neural network.

19. The system of claim 1, wherein:
the contrastive module comprises a contrastive neural network; and
the update trainable parameters further comprises updating trainable parameters of the contrastive neural network.

20. (canceled)

21. A method for training a neural network to classify bio-signal data, the method comprising:
receiving training bio-signal data from one or more subjects comprising labeled training bio-signal data and unlabeled training bio-signal data;
defining one or more sets of time windows within the training bio-signal data, each set comprising a first anchor window and a sampled window;
for at least one set of the one or more sets:
determining a determined set representation based in part on the relative position of the first anchor window and the sampled window;
extracting a feature representation of the first anchor window and a feature representation of the sampled window using an embedder neural network;
aggregating the feature representations using a contrastive module;
predicting a predicted set representation using the aggregated feature representations;
wherein the set representation denotes likely label correspondence between the first anchor window and the sampled window; and
updating trainable parameters of the embedder neural network to minimize a difference between the determined set representation of the at least one set and the predicted set representation of the at least one set; and
labeling the unlabeled training bio-signal data using a classifier, the labeled training bio-signal data, and the embedder neural network.

22. The method of claim 9, further comprising:
receiving user bio-signal data from a user using a bio-signal sensor; and
labeling the user bio-signal data using the embedder neural network and the classifier.

23. (canceled)

24. (canceled)

25. The method of claim 9, wherein:
the one or more sets of time windows comprise one or more pairs of time windows;
the at least one set of the one or more sets comprises at least one pair of the one or more pairs;
the determining a determined set representation based in part on the relative position of the first anchor window and the sampled window comprises:
defining a positive context region and negative context region surrounding the first anchor window;
determining if the sampled window is within the positive context region or negative context region.

26. The method of claim 11, wherein:
the determining a determined set representation based in part on the relative position of the first anchor window and the sampled window further comprises:
rejecting the at least one pair if the sampled window is not within the positive context region or the negative context region.

27. The method of claim 9, wherein:
the one or more sets of time windows comprise one or more triplets of time windows, each triplet further comprising a second anchor window, wherein the second anchor window is within a positive context region surrounding the first anchor window;
the at least one set of the one or more sets comprises at least one triplet of the one or more triplets;
the determining a determined set representation based in part on the relative position of the first anchor window and the sampled window comprises:
determining a temporal order of the first anchor window, the sampled window, and the second anchored window;
the extract a feature representation of the first anchor window and a feature representation of the sampled window using an embedder neural network further comprises extracting a feature representation of the second anchor window.

28. (canceled)

29. The method of claim 9, wherein:
the first anchor window comprises a series of consecutive anchor windows;
the sampled window comprises a series of consecutive sampled windows, wherein the series of consecutive sampled windows is adjacent to the series of consecutive anchor windows;
the set further comprises a set of negative sample windows;
the determining a determined set representation based in part on the relative position of the first anchor window and the sampled window comprises determining that a given sampled window is in the series of sampled windows;
the extracting a feature representation of the first anchor window and a feature representation of the sampled window using an embedder neural network further comprises:
extracting a feature representation of each anchor window of the series of consecutive anchor windows;
extracting a feature representation of each sampled window of the series of consecutive sampled windows; and
extracting a feature representation of each negative sample window of the set of negative sample windows;
the aggregating the feature representations comprises:
embedding the feature representation of each anchor window of the series of anchor windows using an autoregressive embedder;
aggregating the embedded anchor series, a given feature representation of a given sampled window of the series of sampled windows, and one or more given feature representations of one or more given negative sample windows of the set of negative sample windows;
the predicting a predicted set representation comprises predicting which of the given feature representations corresponds to the given feature representation of the sampled window of the series of sampled windows.

30. (canceled)

31. (canceled)

32. (canceled)

33. (canceled)

34. (canceled)

35. The method of claim 9, further comprising:
uploading the embedder neural network to a server.

36. The system of claim 9, wherein:
the contrastive module comprises a contrastive neural network; and
the updating trainable parameters further comprises updating trainable parameters of the contrastive neural network.

37. The method of claim 9, wherein:
the classifier comprises a classifier neural network; and
the update trainable parameters comprises updating trainable parameters of the classifier neural network.

38. (canceled)

39. (canceled)

40. (canceled)

41. A system for training a neural network to classify bio-signal data, the system comprising:
a memory configured to store training bio-signal data from one or more subjects, wherein the training bio-signal data comprises labeled training bio-signal data and unlabeled training bio-signal data;
a training computing apparatus configured to:
receive the training bio-signal data from the memory;
define one or more sets of time windows within the training bio-signal data, each set comprising a series of consecutive anchor windows, a series of consecutive sampled windows, and a set of negative sample windows, and wherein the series of consecutive sampled windows is adjacent to the series of consecutive anchor windows;
for at least one set of the one or more sets:
extract a feature representation of each anchor window of the series of consecutive anchor windows, a feature representation of each sampled window of the series of consecutive sampled windows, and a feature representation of each negative sample window of the set of negative sample windows using an embedder neural network;
embed the feature representation of each anchor window of the series of anchor windows using an autoregressive embedder;
aggregate the embedded feature representation of each anchor window, a given feature representation of a given sampled window, and one or more given feature representations of one or more given negative sample windows using a contrastive module;
predict which of the given sampled window and the one or more given negative sample windows is the given sampled window based on the aggregated feature representations;
update trainable parameters of the embedder neural network to minimize predictions that predict the one or more given negative sample windows is the given sampled window;
label the unlabeled training bio-signal data using a classifier, the labeled training bio-signal data, and the embedder neural network.

42. A system for classifying bio-signal data, the system comprising:
a memory configured to store bio-signal data from one or more subjects;
a computing apparatus configured to:
receive the bio-signal data from the memory;
define one or more sets of time windows within the bio-signal data, each set comprising a first anchor window and a sampled window;
for at least one set of the one or more sets:
determine a determined set representation based in part on the relative position of the first anchor window and the sampled window;
extract a feature representation of the first anchor window and a feature representation of the sampled window using an embedder neural network;
aggregate the feature representations using a contrastive module;
predict a predicted set representation using the aggregated feature representations; and
update trainable parameters of the embedder neural network to minimize a difference between the determined set representation of the at least one set and the predicted set representation of the at least one set;
correspond at least one time window within the bio-signal data with at least one other time window within the bio-signal data based on the feature representation of the at least one time window and the feature representation of the at least one other time window using the trained embedder neural network; and
present corresponded time windows.

43. (canceled)

44. (canceled)

45. An apparatus for classifying bio-signal data, the apparatus comprising:
a bio-signal sensor configured to receive bio-signal data from a subject, the bio-signal data comprising unlabeled bio-signal data;
a computing apparatus configured to:
receive the bio-signal data from the subject;
define one or more sets of time windows within the bio-signal data, each set comprising a first anchor window and a sampled window;
for at least one set of the one or more sets:
determine a determined set representation based in part on the relative position of the first anchor window and the sampled window;
extract a feature representation of the first anchor window and a feature representation of the sampled window using an embedder neural network;
aggregate the feature representations using a contrastive module;
predict a predicted set representation using the aggregated feature representations;
wherein the set representation denotes likely label correspondence between the first anchor window and the sampled window; and
update trainable parameters of the embedder neural network to minimize a difference between the determined set representation of the at least one set and the predicted set representation of the at least one set;
present the bio-signal data to a user;
receive at least one label from the user to generate one or more labeled windows within the bio-signal data;
label the unlabeled bio-signal data using a classifier, the one or more labeled windows, and the embedder neural network.

46. (canceled)
